(19) United States
(12) Patent Application Publication
(10) Pub. No.: US 2014/0371096 A1
(43) Pub. Date: Dec. 18, 2014

(54) COMPOSITIONS AND METHODS FOR CHARACTERIZING THYROID NEOPLASIA

(71) Applicant: The Johns Hopkins University, Baltimore, MD (US)

(72) Inventors: Christopher B. Umbricht, Baltimore, MD (US); Leslie Cope, Baltimore, MD (US); Yan Liu, Beijing (CN); Martha A. Zeiger, Bethesda, MD (US)

(73) Assignee: THE JOHNS HOPKINS UNIVERSITY, Baltimore, MD (US)

(21) Appl. No.: 14/363,901

(22) PCT Filed: Dec. 10, 2012
(86) PCT No.: PCT/US12/68811

§ 371 Date: Jun. 9, 2014

Related U.S. Application Data
(60) Provisional application No. 61/568,923, filed on Dec. 9, 2011.

(51) Int. Cl. C12Q 1/68

(52) U.S. Cl. CPC C12Q 1/6827; C12Q 1/686; G01N 2800/7028
USPC 506/9; 435/6.12; 435/6.11

(57) ABSTRACT

The present invention features compositions and methods for characterizing thyroid lesions (e.g., benign follicular adenomas (FAs), papillary thyroid carcinomas (PTC) and follicular variant papillary thyroid carcinomas (FVPTCs)).

### CROSS-REFERENCE TO RELATED APPLICATION

This application claims the benefit of the following U.S. Provisional Application No. 61/568,923, filed Dec. 9, 2011, the entire contents of which are incorporated herein by reference.

### STATEMENT OF RIGHTS TO INVENTIONS MADE UNDER FEDERALLY SPONSORED RESEARCH

This work was supported by the following grant from the National Institutes of Health, Grant No: R01 CA107247-04. The government has certain rights in the invention.

### BACKGROUND OF THE INVENTION

Fine needle aspiration (FNA) is currently the best diagnostic tool for the pre-operative evaluation of a thyroid nodule, but it is often inconclusive as a guide for subsequent surgical management because 15-20% of fine needle aspirations yield indeterminate results. Recent studies have demonstrated that detecting mutations in BRAF, RAS, RET/PTC, and PAX8/PPARy in clinical fine needle aspiration samples contributes to the diagnostic accuracy of fine needle aspiration cytology. Unfortunately, current assays are still insufficiently sensitive and specific.

Genetic gains and losses in thyroid cancers have been studied. Although DNA copy number changes are frequent in benign follicular adenomas, DNA copy number changes and large chromosomal aberrations are much less common in papillary thyroid carcinomas (PTC) and follicular variant papillary thyroid carcinomas (FVPTCs). FVPTCs and PTCs are particularly difficult to diagnose because morphological classification is subject to significant inter-observer and even intra-observer variation. Characteristic objective measures for diagnosing such tumors is urgently required.

### SUMMARY OF THE INVENTION

As described below, the present invention features compositions and methods for characterizing thyroid lesions (e.g., benign follicular adenomas (FAs), papillary thyroid carcinomas (PTC) and follicular variant papillary thyroid carcinomas (FVPTCs)).

In one aspect, the present invention provides a method for molecularly characterizing a thyroid lesion, the method including detecting in a biological sample of the lesion characteristic DNA copy number variation at one or more of chromosomes 7, 12, and 22, thereby characterizing the lesion as having benign or malignant potential.

In another aspect, the present invention provides a method for characterizing a thyroid lesion, the method including detecting in a biological sample of the lesion characteristic DNA copy number variation at one or more of chromosomes 7, 12, and 22 by one or more of techniques such as, for example, SNP array analysis, PCR analysis, hybridization, fluorescence in situ hybridization, quantitative Real-time genomic PCR analysis, gene expression array analysis, or transcriptome array analysis, thereby characterizing the lesion as having benign or malignant potential.

In another aspect, the present invention provides a method for molecularly characterizing a thyroid lesion, the method including detecting in a biological sample of the lesion characteristic DNA copy number variation at one or more of chromosomes 7, 12, and 22, thereby characterizing the lesion as a benign follicular adenoma, a classic papillary thyroid carcinoma or a follicular variant papillary thyroid carcinoma.

In another aspect, the present invention provides a method for distinguishing a follicular adenoma from other thyroid lesions, the method including detecting in a thyroid lesion a segmental amplification in chromosomes 7 and 12, such that the presence of said amplification at chromosomes 7 and/or 12 is indicative that the lesion is a follicular adenoma.

In yet another aspect, the present invention provides a method for distinguishing adenomatoid nodules or follicular variant papillary thyroid carcinoma from other thyroid lesions, the method comprising detecting in a thyroid lesion a chromosome 12 amplification, such that the presence of the chromosome 12 amplification is indicative of adenomatoid nodules or follicular variant papillary thyroid carcinoma.

In various embodiments of any of the above-delineated aspects, the method may identify a characteristic DNA copy number variation that could not be identified by karyotyping.

In various embodiments of any of the above-delineated aspects, the method may further include detecting a mutation in a Ras gene. In various additional embodiments, the mutation may be H-ras or N-ras.

In various embodiments of any of the above-delineated aspects, the method may further include detecting an increase in telomerase expression or activity. In various additional embodiments, telomerase activity may be detected in an HTERT assay.

In various embodiments of any of the above-delineated aspects, the molecular characterization is not by karyotyping.

In various embodiments of any of the above-delineated aspects, detection of the copy number variation may be by one or more techniques such as, for example, SNP array analysis, PCR analysis, hybridization, fluorescence in situ hybridization, quantitative Real-time genomic PCR analysis, gene expression array analysis, or transcriptome array analysis.

In various embodiments of any of the above-delineated aspects, the characteristic DNA copy number variation is a segmental amplification at chromosome 12 that is indicative of a follicular adenoma.

In various embodiments of any of the above-delineated aspects, the method distinguishes a follicular adenoma from a classic papillary thyroid carcinoma or a follicular variant papillary thyroid carcinoma.

In various embodiments of any of the above-delineated aspects, the characteristic DNA copy number variation is chromosome 12 amplification that identifies the lesion as being benign or as having no or little malignant potential.

In various embodiments of any of the above-delineated aspects, amplification at chromosome 12 is detected by measuring the expression or activity of any one or more markers selected from the group consisting of NDUFA12, NR2C1, FGD6, VEZT, MIR331, RPL29P26, LOC729457, METAP2, USP44, CD163L1, LOC727815, BICD1, FGD4, DNM1L, YARS2, UTP20, ARL1, SPIC, WNK1, DRAM, RAD52, HSPD1P12, CERS5, LIMA1, MYBPC1, CHPT1, SYCP3, PKP2, CCDC53, HAUS6, PLIN2, LOC729925, YPEL2, DHX40, CLTC, PTRH2, TMEM49, MIR21, TUBD1, PLIN2, RPS6 KB1, HEATR6, LOC645638, LOC653653, LOC650609, CA4, USP32, SCARNA20, C17orf64, and APPBP2.

In various embodiments of any of the above-delineated aspects, amplification at chromosome 12 is detected by measuring the expression or activity of any one or more markers selected from the group consisting of NDUFA12, NR2C1, FGD6, VEZT, MIR331, RPL29P26, LOC729457, METAP2, USP44, and CD163L1.

In various embodiments of any of the above-delineated aspects, amplification at chromosome 12 is detected by measuring the expression or activity of any one or more markers selected from the group consisting of NDUFA12, NR2C1, FGD6, VEZT and GDF3.

In various embodiments of any of the above-delineated aspects, the characteristic DNA copy number variation is a chromosome 22 deletion, and presence of the deletion is indicative of a premalignant state leading to invasive disease.

In various embodiments of any of the above-delineated aspects, the biological sample is a tissue sample, biopsy sample, or fine needle aspirant.

In various embodiments of any of the above-delineated aspects, RNA or genomic DNA may be isolated from the sample prior to analysis.

In various embodiments of any of the above-delineated aspects, detection of the amplification on chromosome 12 indicates that said follicular adenoma is unlikely to progress to thyroid cancer.

The invention provides characterizing thyroid lesions using DNA copy number variations to determine their benign or malignant potential. Compositions and articles defined by the invention were isolated or otherwise manufactured in connection with the examples provided below. Other features and advantages of the invention will be apparent from the detailed description, and from the claims.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “NADH dehydrogenase (ubiquinone) 1 alpha subcomplex, 12 (NDUFA12) nucleic acid molecule” is meant a polynucleotide encoding a NDUFA12 polypeptide. See, NCBI Gene ID 55967. Exemplary NDUFA12 nucleic acid molecules are provided at NCBI Accession Nos. NM_{—}001258338.1 and NM_{—}018838.4, as well as below:

>gi|385275075|ref|NM_001258338.1| Homo sapiens NADH dehydrogenase
(ubiquinone) 1 alpha subcomplex, 12 (NDUFA12), transcript variant 2,
mRNA
GGCGCACCCGGGAGGCGGGGCCAGCGAGGCAAGATGGAGTTAGTGCAGGTCCTGAAACGCGGGCTGCAGC
AGATCACCGGCCACGGCGGTCTCCGAGGCTATCTACGGGTTTTTTTCAGGACAAATGATGCGAAGGTTGG
TACATTAGTGGGGGAAGACAAATATGGAAACAAATACTATGAAGACAACAAGCAATTTTTTGGCATCGTT
GGCTTCACAGTATGACTGATGATCCTCCAACAACAAAACCACTTACTGCTCGTAAATTCATTTGGACGAA
CCATAAATTCAACGTGACTGGCACCCCAGAACAATATGTACCTTATTCTACCACTAGAAAGAAGATTCAG
GAGTGGATCCCACCTTCAACACCTTACAAGTAAAGACAATGAAGAACAGTTGAAACATGCAAAATATGGA
GCTTTTCATGTAATTACTCTTTTACTGTTTACCATTCACTATAATTCACAATTAAAATTGTGTGACTAAA
CAATGAAAAAAAAA
>gi|385275074|ref|NM_018838.4| Homo sapiens NADH dehydrogenase
(ubiquinone) 1 alpha subcomplex, 12 (NDUFA12), nuclear gene encoding
mitochondrial protein, transcript variant 1, mRNA
GGCGCACCCGGGAGGCGGGGCCAGCGAGGCAAGATGGAGTTAGTGCAGGTCCTGAAACGCGGGCTGCAGC
AGATCACCGGCCACGGCGGTCTCCGAGGCTATCTACGGGTTTTTTTCAGGACAAATGATGCGAAGGTTGG
TACATTAGTGGGGGAAGACAAATATGGAAACAAATACTATGAAGACAACAAGCAATTTTTTGGCCGTCAC
CGATGGGTTGTATATACTACTGAAATGAATGGCAAAAACACATTCTGGGATGTGGATGGAAGCATGGTGC
CTCCTGAATGGCATCGTTGGCTTCACAGTATGACTGATGATCCTCCAACAACAAAACCACTTACTGCTCG
TAAATTCATTTGGACGAACCATAAATTCAACGTGACTGGCACCCCAGAACAATATGTACCTTATTCTACC
ACTAGAAAGAAGATTCAGGAGTGGATCCCACCTTCAACACCTTACAAGTAAAGACAATGAAGAACAGTTG
AAACATGCAAAATATGGAGCTTTTCATGTAATTACTCTTTTACTGTTTACCATTCACTATAATTCACAAT
TAAAATTGTGTGACTAAACAATGAAAAAAAAA

By “nuclear receptor subfamily 2, group C, member 1 (NR2C1) nucleic acid molecule” is meant a polynucleotide encoding a NR2C1 polypeptide. See, NCBI Gene ID 7181. Exemplary NR2C1 nucleic acid molecules are provided at NCBI Accession Nos. NM_{—}003297.3, NM_{—}001032287.2, and NM_{—}001127362.1, as well as below:

>gi|384475525|ref|NM_003297.3| Homo sapiens nuclear receptor subfamily
2, group C, member 1 (NR2C1), transcript variant 1, mRNA
GCTTCTCCCCGTTGCTAATGCGCAGGCGCTGGCGGGATAGCGCGCCGCCGAGCCGAGAAAGAGGTCACGA
ACTCTGACCCCCCAGAAATACCCAAACACAGAAAGCTCTCTCCGCCGTGAATCTCGATCCCACATCCCGT
CGGCTTTCTTCAACCTCTCTTCCCGGAGCGCCCCCCAATCCACGAGTGGCAGCCGCGGGACTGTCGCGTC
GGCGCCCGACGCCGGAGTCAGCAGGGCGCAAAAGCGCCGGTAGATCATGGCAACCATAGAAGAAATTGCA
CATCAAATTATTGAACAACAGATGGGAGAGATTGTTACAGAGCAGCAAACTGGGCAGAAAATCCAGATTG
TGACAGCACTTGATCATAATACCCAAGGCAAGCAGTTCATTCTGACAAATCACGACGGCTCTACTCCAAG
CAAAGTCATTCTGGCCAGGCAAGATTCCACTCCGGGAAAAGTTTTCCTTACAACTCCAGATGCAGCAGGT
GTCAACCAGTTATTTTTTACCACTCCTGATCTGTCTGCACAACACCTGCAGCTCCTAACAGATAATTCTC
CAGACCAAGGACCAAATAAGGTTTTTGATCTTTGCGTAGTATGTGGAGACAAAGCATCAGGACGTCATTA
TGGAGCAGTAACTTGTGAAGGCTGCAAAGGATTTTTTAAAAGAAGCATCCGAAAAAATTTAGTATATTCA
TGTCGAGGATCAAAGGATTGTATTATTAATAAGCACCACCGAAACCGCTGTCAATACTGCAGGTTACAGA
GATGTATTGCGTTTGGAATGAAGCAAGACTCTGTCCAATGTGAAAGAAAACCCATTGAAGTATCACGAGA
AAAATCTTCCAACTGTGCCGCTTCAACAGAAAAAATCTATATCCGAAAGGACCTTCGTAGCCCATTAACT
GCAACTCCAACTTTTGTAACAGATAGTGAAAGTACAAGGTCAACAGGACTGTTAGATTCAGGAATGTTCA
TGAATATTCATCCATCTGGAGTAAAAACTGAGTCAGCTGTGCTGATGACATCAGATAAGGCTGAATCATG
TCAGGGAGATTTAAGTACATTGGCCAATGTGGTTACATCATTAGCGAATCTTGGAAAAACTAAAGATCTT
TCTCAAAATAGTAATGAAATGTCTATGATTGAAAGCTTAAGCAATGATGATACCTCTTTGTGTGAATTTC
AAGAAATGCAGACCAACGGTGATGTTTCAAGGGCATTTGACACTCTTGCAAAAGCATTGAATCCTGGAGA
GAGCACAGCCTGCCAGAGCTCAGTAGCGGGCATGGAAGGAAGTGTACACCTAATCACTGGAGATTCAAGC
ATAAATTACACCGAAAAAGAGGGGCCACTTCTCAGCGATTCACATGTAGCTTTCAGGCTCACCATGCCTT
CTCCTATGCCTGAGTACCTGAATGTGCACTACATTGGGGAGTCTGCCTCCAGACTGCTGTTCTTATCAAT
GCACTGGGCACTTTCGATTCCTTCTTTCCAGGCTCTAGGGCAAGAAAACAGCATATCACTGGTGAAAGCT
TACTGGAATGAACTTTTTACTCTTGGTCTTGCCCAGTGCTGGCAAGTGATGAATGTAGCAACTATATTAG
CAACATTTGTCAATTGTCTTCACAATAGTCTTCAACAAGATAAAATGTCAACAGAAAGAAGAAAATTATT
GATGGAGCACATCTTCAAACTACAGGAGTTTTGTAACAGCATGGTTAAACTCTGCATTGATGGATACGAA
TATGCCTACCTGAAGGCAATAGTACTCTTCAGTCCAGATCATCCAAGCCTAGAAAACATGGAACAGATAG
AGAAATTTCAGGAAAAGGCTTATGTGGAATTCCAAGATTATATAACCAAAACATATCCAGATGACACCTA
CAGGTTATCCAGACTACTACTCAGATTGCCAGCTTTAAGACTGATGAATGCTACCATCACTGAAGAATTG
TTTTTCAAAGGTCTCATTGGCAATATACGAATTGACAGTGTTATCCCACATATTTTGAAAATGGAGCCTG
CAGATTATAACTCTCAAATAATTGGTCACAGCATTTGAAAACTGTGACTGCAGTGCTGTAAACTTAACTG
TTCTTTGCCAGAACACAAGACACCAAATTGAACTCACTGCTTTTGAGGCATCTGGAAATTTTTACTTTAA
AAAGTAACCAGAATCCAAGGTATTTTTATTTTAGCTTCCCTTAAGAATTTTTGAAGTGACTGGGCAGGCA
GCAGAAATTAAATGAATTTTTCTTCCTGATTCCTTTAAATGAATATGAAACACTACAAATTTATTCTTGG
TGAAGATGATACCTGAAGCTGTCACCTCTTGATTATCTAAACTAAGCGCTCATTCTATTTTATAAAACAA
ATAAATTAGTCTCTTTTTTCTGAATTGTGTTCTAGTCATATTTAACTTCATTATGAACTAGTAAAAATAC
TTAATGGTCAGAAATCCCTAAGGAGTTAGTTCCTTGCATTTTACTCTGCCATAATAATTTTTGTTTAATT
ACCATATCAAAATAAGATTATTTTATGCTTACTGGTATAATGACAGTATTAGAACTATAGGAAATAATTG
AATACATATTTTTTGTCTTCTCTAAATATCATGGTGTCCCTTAGCATATACTACTCTCATTGCTGGCAGT
GAGACAGGCCATTCATGATCTTAAGAGTTGCCATTTTTAATGTATATTATTAGTTACAAGCACTTTATAT
AGCAGAAAATTGTTTTTGAGAATAAGCTAGTGTTGATATTTTAATATTTTTAGCTTACTGCTCGTGTTTT
TGTTTTTGTTTTCGTTTATAGAGGTGGGTTTCACTGTTGCCCAGGCTGGTCTCAAACTCCTGGGCTCAAG
TGATCCTGCCTCAGCCTCCCAAAGTACTGGGATTACAGGCGCGTGCCACCGTGCCTGGCCTACTGCTGTC
TTTGAAAATAATAGAGACTAGCCAGGTGTAGTGGCTCATGCCTATAATCCCAGCACTTTGGGAGGCTGAG
GCAGGCAGATTGCTTGAGCTCAGGAGTTCGAGACCAGCCTGGGCAATATAGCAAGACCTCGTCTCTGTAA
AAAGAAAGAAAGTAATAAAGACTAATTGAGCCCAAAATGTTTCACTATTTCAAAAAAGATATTTAAATTG
TTGCTCTTTCATTCCATAAAAAGGATCTGATCTCTCTCCCACTTTTCTGACCTGAGTTAGAGCTTCCCAA
ACCTGTCATGTATGGGTTTTAGCCAATTTCTTTTAGATCACTAAAAAAACTCACCCAATATGTCAAATAA
TGGATTTATCATAGCCAGTACATGTTCTCAAGGCAAGTTTAAACATTATTTTGAAGCTATTGATAATTTT
TTAAAATAAAGAAATATTCACTGATTTTTTTCACTGTAAAGCACGGGAGGGCTGCTTTAACAACAGTATA
AGAATCAGCCTGAAGCCTTGTTACTGCTACAACAAATTCATTTTAGACTCCTCGGATGTCTTCCACAGTA
ATTTATTCTTTTAGCAAACCTGATACTGATAACTGTTTCTTTGCTTTGATTTCTTGATGAATTATTTTGG
TATGTTTGTTGATTTTTAAAGCAAACACGGATAATGCACTCAGAGTACATTTTTTGTAAAGATTTTTGCA
ATAGAAGAAAAGTGAAGTTTTTGTGGGGATGTGGATTTTATTGCTTACTACTTTATAGTAATCAAAAGTT
TGAAAATATCAACTTACAGTCTTTACCAGTTTACTAAGGGAAACTTTTTTCCCTATTTAAAACATGATCT
TAGTCAACAATTTTATTTATAATTATCAGCTAAATTACATTTAGTATAATACTCAAATGGAAAAATCAGT
AGTTTATACCTTTATAAATACAGTTTAGTAAGCCAAGGAATCAGGGAAATAATCCTTTAAAATAATGTAC
TAATAGTTAAGATGTTTCAGGTGTTTTTTCTGATTAAATTTGCTACTATATTTGGAAGACTTTAAAACTA
TATTAAAATGTGACTTGCATTACAAATTTCTGTGTCTTACCAGTATATTTGTAAATATATTATTCATTTT
CCTTTTCA
>gi|189491737|ref|NM_001032287.2| Homo sapiens nuclear receptor
subfamily 2, group C, member 1 (NR2C1), transcript variant 2, mRNA
GCTTCTCCCCGTTGCTAATGCGCAGGCGCTGGCGGGATAGCGCGCCGCCGAGCCGAGAAAGAGGTCACGA
ACTCTGACCCCCCAGAAATACCCAAACACAGAAAGCTCTCTCCGCCGTGAATCTCGATCCCACATCCCGT
CGGCTTTCTTCAACCTCTCTTCCCGGAGCGCCCCCCAATCCACGAGTGGCAGCCGCGGGACTGTCGCGTC
GGCGCCCGACGCCGGAGTCAGCAGGGCGCAAAAGCGCCGGTAGATCATGGCAACCATAGAAGAAATTGCA
CATCAAATTATTGAACAACAGATGGGAGAGATTGTTACAGAGCAGCAAACTGGGCAGAAAATCCAGATTG
TGACAGCACTTGATCATAATACCCAAGGCAAGCAGTTCATTCTGACAAATCACGACGGCTCTACTCCAAG
CAAAGTCATTCTGGCCAGGCAAGATTCCACTCCGGGAAAAGTTTTCCTTACAACTCCAGATGCAGCAGGT
GTCAACCAGTTATTTTTTACCACTCCTGATCTGTCTGCACAACACCTGCAGCTCCTAACAGATAATTCTC
CAGACCAAGGACCAAATAAGGTTTTTGATCTTTGCGTAGTATGTGGAGACAAAGCATCAGGACGTCATTA
TGGAGCAGTAACTTGTGAAGGCTGCAAAGGATTTTTTAAAAGAAGCATCCGAAAAAATTTAGTATATTCA
TGTCGAGGATCAAAGGATTGTATTATTAATAAGCACCACCGAAACCGCTGTCAATACTGCAGGTTACAGA
GATGTATTGCGTTTGGAATGAAGCAAGACTCTGTCCAATGTGAAAGAAAACCCATTGAAGTATCACGAGA
AAAATCTTCCAACTGTGCCGCTTCAACAGAAAAAATCTATATCCGAAAGGACCTTCGTAGCCCATTAACT
GCAACTCCAACTTTTGTAACAGATAGTGAAAGTACAAGGTCAACAGGACTGTTAGATTCAGGAATGTTCA
TGAATATTCATCCATCTGGAGTAAAAACTGAGTCAGCTGTGCTGATGACATCAGATAAGGCTGAATCATG
TCAGGGAGATTTAAGTACATTGGCCAATGTGGTTACATCATTAGCGAATCTTGGAAAAACTAAAGATCTT
TCTCAAAATAGTAATGAAATGTCTATGATTGAAAGCTTAAGCAATGATGATACCTCTTTGTGTGAATTTC
AAGAAATGCAGACCAACGGTGATGTTTCAAGGGCATTTGACACTCTTGCAAAAGCATTGAATCCTGGAGA
GAGCACAGCCTGCCAGAGCTCAGTAGCGGGCATGGAAGGAAGTGTACACCTAATCACTGGAGATTCAAGC
ATAAATTACACCGAAAAAGAGGGGCCACTTCTCAGCGATTCACATGTAGCTTTCAGGCTCACCATGCCTT
CTCCTATGCCTGAGTACCTGAATGTGCACTACATTGGGGAGTCTGCCTCCAGACTGCTGTTCTTATCAAT
GCACTGGGCACTTTCGATTCCTTCTTTCCAGGCTCTAGGGCAAGAAAACAGCATATCACTGGTGAAAGCT
TACTGGAATGAACTTTTTACTCTTGGTCTTGCCCAGTGCTGGCAAGTGATGAATGTAGCAACTATATTAG
CAACATTTGTCAATTGTCTTCACAATAGTCTTCAACAAGCAGAGGGGTAATCACCTTAAAATGTCATCAA
AAATAGATCTACTAGAAGGCAGCATCACATTCCCATCTTACTTATGGACTCCTACCCCTGGTTCATGTCT
TATATGCCTGTAATGGTTATAAAGCCTACCTTCAGGAAAGCTATGGTTGACTAATTACTAATGGATGGGT
TTTAAACATGTCCCTCTACAATAAATTAAAATCTTTATTGTAAAACTTTAAAAAAAAAAAAAAAAAAAAA
AAAAAAAAAAAAA
>gi|189491765|ref|NM_001127362.1| Homo sapiens nuclear receptor
subfamily 2, group C, member 1 (NR2C1), transcript variant 3, mRNA
GCTTCTCCCCGTTGCTAATGCGCAGGCGCTGGCGGGATAGCGCGCCGCCGAGCCGAGAAAGAGGTCACGA
ACTCTGACCCCCCAGAAATACCCAAACACAGAAAGCTCTCTCCGCCGTGAATCTCGATCCCACATCCCGT
CGGCTTTCTTCAACCTCTCTTCCCGGAGCGCCCCCCAATCCACGAGTGGCAGCCGCGGGACTGTCGCGTC
GGCGCCCGACGCCGGAGTCAGCAGGGCGCAAAAGCGCCGGTAGATCATGGCAACCATAGAAGAAATTGCA
CATCAAATTATTGAACAACAGATGGGAGAGATTGTTACAGAGCAGCAAACTGGGCAGAAAATCCAGATTG
TGACAGCACTTGATCATAATACCCAAGGCAAGCAGTTCATTCTGACAAATCACGACGGCTCTACTCCAAG
CAAAGTCATTCTGGCCAGGCAAGATTCCACTCCGGGAAAAGTTTTCCTTACAACTCCAGATGCAGCAGGT
GTCAACCAGTTATTTTTTACCACTCCTGATCTGTCTGCACAACACCTGCAGCTCCTAACAGATAATTCTC
CAGACCAAGGACCAAATAAGGTTTTTGATCTTTGCGTAGTATGTGGAGACAAAGCATCAGGACGTCATTA
TGGAGCAGTAACTTGTGAAGGCTGCAAAGGATTTTTTAAAAGAAGCATCCGAAAAAATTTAGTATATTCA
TGTCGAGGATCAAAGGATTGTATTATTAATAAGCACCACCGAAACCGCTGTCAATACTGCAGGTTACAGA
GATGTATTGCGTTTGGAATGAAGCAAGACTCTGTCCAATGTGAAAGAAAACCCATTGAAGTATCACGAGA
AAAATCTTCCAACTGTGCCGCTTCAACAGAAAAAATCTATATCCGAAAGGACCTTCGTAGCCCATTAACT
GCAACTCCAACTTTTGTAACAGATAGTGAAAGTACAAGGTCAACAGGACTGTTAGATTCAGGAATGTTCA
TGAATATTCATCCATCTGGAGTAAAAACTGAGTCAGCTGTGCTGATGACATCAGATAAGGCTGAATCATG
TCAGGGAGATTTAAGTACATTGGCCAATGTGGTTACATCATTAGCGAATCTTGGAAAAACTAAAGATCTT
TCTCAAAATAGTAATGAAATGTCTATGATTGAAAGCTTAAGCAATGATGATACCTCTTTGTGTGAATTTC
AAGAAATGCAGACCAACGGTGATGTTTCAAGGGCATTTGACACTCTTGCAAAAGCATTGAATCCTGGAGA
GAGCACAGCCTGCCAGAGCTCAGTAGCGGGCATGGAAGGAAGTGTACACCTAATCACTGGAGATTCAAGC
ATAAATTACACCGAAAAAGAGGGGCCACTTCTCAGCGATTCACATGTAGCTTTCAGGCTCACCATGCCTT
CTCCTATGCCTGAGTACCTGAATGTGCACTACATTGGGGAGTCTGCCTCCAGACTGCTGTTCTTATCAAT
GCACTGGGCACTTTCGATTCCTTCTTTCCAGGCTCTAGGGCAAGAAAACAGCATATCACTGGTGAAAGCT
TACTGGAATGAACTTTTTACTCTTGGTCTTGCCCAGTGCTGGCAAGTGATGAATGTAGCAACTATATTAG
CAACATTTGTCAATTGTCTTCACAATAGTCTTCAACAAGATGCCAAGGTAATTGCAGCCCTCATTCATTT
CACAAGACGAGCAATCACTGATTTATAAATGCTTAACTATAGAATGGCTTATGACTACCCAAAACAGTGC
CCCATCAACAAATGGGGAAAATTGCCTTTTGAGCTCAGGAATAATTTATAAATTGGGGACTACCTTTTAG
TTCTTTAGCATATTCTATTTCTTATTGTTTTATATAATTTTTAAATCATTTGCTTCCTCCTTATGTTTAA
CAGCAGAGGGGTAATCACCTTAAAATGTCATCAAAAATAGATCTACTAGAAGGCAGCATCACATTCCCAT
CTTACTTATGGACTCCTACCCCTGGTTCATGTCTTATATGCCTGTAATGGTTATAAAGCCTACCTTCAGG
AAAGCTATGGTTGACTAATTACTAATGGATGGGTTTTAAACATGTCCCTCTACAATAAATTAAAATCTTT
ATTGTAAAACTTTAAAAAAAAAAAAAAAAAAAAAAAAAAAAAAAAAA

By “FYVE, RhoGEF and PH domain containing 6 (FGD6) nucleic acid molecule” is meant a polynucleotide encoding a FGD6 polypeptide, as summarized in NCBI Gene ID 55785. An exemplary FGD6 nucleic acid molecule is provided at NCBI Accession No. NM_{—}018351.3, as well as below:

>gi|154240685|ref|NM_018351.3| Homo sapiens FYVE, RhoGEF and PH domain
containing 6 (FGD6), mRNA
AGTGCTCGCCCGCCCGACCCCGGCGGCTCGCGCCCGGGAGCGCCGCAGGGTCGCTAGAGTCGGCCGCGTC
CTTTGTGTGGCGCTCAGGCTGCGCCGCGGGGCGGCGGGACGGAATGTGGGCGCTGCGGGGGCTTTTCTCT
CCTACCCGAACTGTGGGAACAATGGACTGAAAGGGGAAGATGGATTGAGGGGCCGAGCGGGGAAGCGAGC
TGCACCGGGGAATCATGACTTCTGCAGCCGAGATAAAGAAGCCACCAGTGGCCCCCAAGCCCAAGTTTGT
TGTGGCAAATAATAAGCCAGCCCCACCTCCTATTGCACCTAAACCCGACATTGTGATTTCTAGTGTTCCA
CAGTCGACAAAGAAAATGAAACCAGCAATAGCCCCAAAACCAAAAGTCCTGAAGACCTCACCTGTTCGAG
AGATTGGGCAGTCGCCATCAAGGAAAATCATGTTGAACCTGGAAGGGCATAAACAGGAATTAGCTGAAAG
CACTGACAACTTTAATTGTAAATATGAAGGCAATCAGAGCAATGATTATATTTCACCAATGTGTTCCTGC
AGTTCTGAGTGTATCCATAAGCTGGGCCATAGAGAGAATTTGTGTGTAAAGCAGCTTGTTTTAGAGCCCC
TGGAAATGAATGAAAATTTAGAAAACAGTAAAATTGATGAGACTTTGACTATAAAAACTAGGAGTAAATG
TGATTTGTATGGTGAAAAAGCCAAGAACCAGGGTGGGGTTGTTTTAAAGGCAAGCGTTTTAGAAGAGGAG
CTCAAAGATGCCTTAATACACCAAATGCCACCTTTTATTTCTGCACAGAAGCACAGGCCCACAGACAGCC
CAGAAATGAATGGTGGCTGTAATTCAAATGGACAATTCAGAATTGAATTTGCGGATTTGTCACCTTCCCC
ATCCAGCTTTGAAAAAGTTCCTGATCATCACAGTTGCCACTTACAGCTTCCTAGTGATGAATGTGAACAT
TTTGAAACTTGCCAGGATGACAGTGAAAAAAGCAATAATTGCTTTCAGTCATCTGAACTAGAGGCTCTGG
AAAATGGGAAAAGGAGTACTTTAATATCTTCAGATGGAGTTAGTAAGAAATCAGAAGTCAAAGACCTTGG
TCCCTTAGAAATTCATTTAGTACCATATACCCCAAAATTTCCAACTCCCAAGCCCAGAAAGACACGAACT
GCTCGTCTGTTACGCCAAAAGTGTGTAGATACTCCTAGTGAAAGCACTGAAGAACCGGGGAATTCAGACA
GTAGCTCTTCCTGTCTTACTGAAAATAGTTTGAAAATCAATAAAATCAGTGTTCTGCATCAGAATGTTTT
GTGTAAGCAGGAACAGGTGGATAAAATGAAGCTAGGAAATAAAAGTGAATTGAATATGGAATCCAACAGT
GATGCACAGGACTTAGTCAATTCACAGAAAGCCATGTGTAATGAAACAACTTCCTTTGAAAAAATGGCAC
CTTCTTTTGATAAAGACTCTAATTTGAGTTCTGACAGCACAACTGTAGATGGTTCTAGTATGTCGCTTGC
TGTGGACGAAGGGACCGGTTTTATAAGATGTACTGTATCTATGAGCCTGCCTAAGCAGCTCAAATTAACT
TGCAATGAACATTTGCAATCTGGGAGAAACCTGGGAGTTTCTGCCCCTCAAATGCAAAAGGAATCTGTTA
TAAAAGAGGAAAATTCTCTACGAATTGTCCCCAAAAAACCTCAAAGACATAGCTTGCCTGCTACAGGAGT
GCTTAAAAAGGCTGCCTCCGAGGAGCTTTTGGAAAAAAGTTCTTATCCTTCAAGTGAAGAAAAAAGTTCA
GAGAAGAGTCTAGAAAGAAATCACCTTCAGCATTTGTGTGCCCAAAACCGTGGTGTGTCATCCTCCTTTG
ATATGCCTAAACGGGCTTCAGAAAAGCCAGTGTGGAAGTTACCTCATCCTATTTTACCCTTTTCAGGGAA
CCCAGAATTCTTAAAGTCTGTCACCGTATCGTCAAACAGTGAGCCTTCAACAGCCCTAACCAAGCCCAGA
GCAAAATCGTTATCTGCTATGGATGTGGAAAAGTGCACTAAGCCTTGCAAAGACTCTACAAAGAAAAACT
CTTTTAAAAAGTTGCTCAGCATGAAACTGTCCATCTGTTTCATGAAGAGTGACTTTCAAAAATTTTGGTC
CAAGAGTAGCCAACTCGGAGACACCACCACAGGCCACCTCTCCAGTGGGGAGCAGAAGGGGATTGAAAGT
GATTGGCAAGGCTTGTTGGTAGGAGAGGAGAAGAGAAGTAAACCCATCAAGGCATATTCCACAGAAAACT
ATAGCCTGGAATCTCAAAAGAAGAGGAAGAAGTCTCGGGGCCAGACCAGTGCAGCTAATGGTCTGAGAGC
TGAGTCTTTGGATGACCAAATGCTCTCCCGGGAGTCATCATCTCAGGCACCTTACAAGTCTGTTACAAGC
CTCTGTGCACCGGAGTATGAAAATATACGCCATTATGAGGAAATACCAGAGTACGAGAACTTGCCATTTA
TTATGGCTATACGGAAAACTCAAGAGTTGGAATGGCAGAATTCCAGCAGCATGGAGGACGCTGATGCAAA
TGTGTATGAGGTAGAAGAGCCGTATGAAGCTCCAGATGGCCAGCTGCAGCTTGGACCCAGACATCAGCAT
TCCAGTTCAGGAGCATCCCAGGAGGAACAGAATGATCTTGGTCTTGGTGACCTTCCCTCTGATGAGGAGG
AAATCATCAACAGTTCTGATGAAGATGATGTCAGCTCTGAGTCAAGTAAAGGAGAGCCTGACCCACTGGA
AGATAAACAGGATGAAGATAATGGAATGAAAAGTAAAGTTCATCATATTGCCAAGGAGATCATGAGCTCA
GAGAAAGTGTTTGTGGATGTGTTAAAACTTTTGCATATTGATTTCCGGGATGCAGTAGCTCATGCTTCCA
GGCAACTTGGGAAACCAGTGATTGAGGACCGGATTCTAAATCAGATCCTATACTACTTGCCTCAGCTGTA
TGAGCTCAACCGGGATCTCTTGAAGGAACTGGAGGAAAGAATGTTGCACTGGACTGAACAACAAAGAATT
GCTGATATCTTTGTAAAGAAGGGACCATATCTAAAAATGTATTCCACATACATCAAAGAATTTGATAAGA
ATATAGCCTTGCTGGATGAACAGTGCAAGAAAAATCCAGGTTTTGCTGCTGTTGTTAGAGAATTTGAGAT
GAGCCCTCGCTGTGCTAATCTGGCCCTCAAGCACTACCTGCTCAAGCCGGTTCAGAGGATCCCCCAGTAC
AGGCTGTTGCTGACAGATTATTTGAAGAATCTCATAGAAGATGCTGGAGATTACAGAGACACTCAAGATG
CCCTTGCTGTTGTTATAGAGGTAGCCAACCACGCCAATGACACCATGAAGCAAGGAGACAACTTTCAGAA
ACTTATGCAAATTCAGTACAGCTTAAATGGACACCATGAAATTGTGCAGCCTGGTCGGGTTTTTCTCAAA
GAAGGAATTCTGATGAAGCTGTCTCGGAAAGTGATGCAACCTCGAATGTTTTTCCTGTTTAATGATGCCC
TGCTGTATACAACACCAGTGCAGTCTGGGATGTATAAACTGAACAACATGCTCTCACTGGCTGGAATGAA
GGTCAGAAAACCTACCCAAGAAGCCTATCAGAATGAATTAAAGATTGAAAGTGTAGAACGTTCCTTCATT
CTCTCAGCCAGTTCTGCCACAGAAAGGGATGAATGGCTAGAAGCGATTTCCAGGGCAATAGAAGAGTATG
CCAAGAAAAGAATCACCTTCTGTCCTAGTAGGAGTCTTGATGAGGCAGACTCAGAAAATAAAGAAGAAGT
TAGTCCTCTTGGATCGAAGGCTCCCATCTGGATTCCTGATACCAGAGCCACAATGTGTATGATCTGCACA
AGCGAATTCACTCTCACCTGGAGACGACACCACTGCCGGGCCTGTGGAAAGATTGTATGCCAAGCTTGTT
CGTCTAATAAGTATGGCTTAGATTACCTGAAAAATCAACCAGCAAGAGTATGTGAACATTGTTTCCAAGA
ACTGCAGAAATTAGATCACCAGCACTCCCCTAGGATTGGATCTCCTGGAAATCACAAATCTCCTTCAAGT
GCCTTATCATCAGTCTTACATAGCATTCCATCAGGGAGGAAACAGAAAAAAATCCCAGCTGCTCTCAAAG
AAGTATCAGCAAACACAGAGGATTCTTCTATGAGTGGCTACTTGTACAGATCAAAGGGCAATAAAAAACC
CTGGAAACACTTTTGGTTTGTCATAAAAAATAAAGTACTATATACATATGCTGCAAGTGAGGACGTGGCC
GCTTTGGAGAGTCAGCCTTTATTAGGATTCACTGTTATTCAAGTTAAAGATGAGAATTCCGAGTCTAAAG
TATTTCAGTTACTGCACAAAAACATGTTATTTTATGTATTCAAAGCAGAGGATGCTCATTCGGCTCAGAA
GTGGATAGAAGCATTTCAGGAAGGCACAATATTGTAGCAGTATTGGTTTCATCTCTTCTGTGATTCCAAA
GAGGTGGAATTTCATCAGAATGGAGTAAATGCAATTCAAAAATTGTATAAAAATGAACACTGCCAAGATA
AAGCCAACCAGACCCTTCATCAAAGAAATTGTTTTGTTAGGTATAAGCAATTTTTAAAAGGTGTTTGTTT
TTTCATTTATGTTATTTATTAAAATTTTGATGTTTACTTAATGGTCAGAATTATTTCTGAGACACACTGA
ATTCTAAAGTACCATTTCTTTAGAGACCAGAAAAACTATCTTAATACTGTATACTGTATTAACTATTCGT
GACATAGTTCACACTGTTTTCTTACCTTACATTGTAACAATCTTACTGGTGGAAAGTCTTTGTAAGGAAA
AAACACATAGCAAGGAGCAAATTTCCACAAAGTGCTTGGTTTAGGAATTGTGATTATTATAAAACTGCTG
ATGAAAAAAATGCATGTCTTTGAATCAATAAACTTGGGTGAATATTTGTATCTTTTAGTGGAAAAACATG
GCCAGCTTCTACCTCAGTAACTGTGAACTGAAATTTCAGTAAATTATCTAAAGTATTTCTGTTGTTAGGT
ACCTCTTTGGCAGGAGTTAATATTACATCATCAAAGAATTATAGCAAAGAGATAGAATCTGAATTTTTTA
AAACTGTGAGTAGGAATGAAGATGTTTTTATTTGCAGAATACCACAAATAACCAACTCTTCCGGCTTTTA
AGTCCAATCTTTTAAAAAATCTACCACTTCGAAACAAACATAAATGTATCATTTTTTAAAATAGCAAAAT
ATAGCAAGCATTATGTCACATAATATTCCCTGCTATTATAAGAGTTCTGAGCCCAAGTCAATGATGATAT
TTGTATCTATAAGTAATGTTACATTTCCAAAAATATTGTGCATTACAAATGGAACTGGAATTACTATATC
AGAAAAGCATAATTATAAGCCAGTAATAACTGAAATTCTATAGTATTCATTTTCAAAAGGTCTTTTTCTG
CCAGTTTGTGATATCCTCCCTCCTAATTAAAAAAAAAAACAACAAATCCTTTCTCTATAAGCAGCTATCA
GCACACCTCCTTAGGAAAGATTTAGATTCATAATTCTGGTGCACTTACTGTTTAACATATGAACTACCTT
GCACATACAATTGTTGATTAGCAGAAGAAAATGAAATAACACTGTGATAAAAGCCATCCCTGATGTTCAC
AATACACAATTTATTAACTAAGTTTAAACTATAAATTATCTTAACTGCCATGAGCGGTGGCTCACACCTA
CAATCTCAGCATTTTGGGAGGCCGAGGCCGTTGGACCACCTGAGGTCAGGAGATCGAGACCAGCCTGGCC
AATATGGTGAAACCCCATCTCTATTAAAAATACAAGAATTAGCCGGTCGTGGTGGTACATGGCTGTAGTC
CTAGCTATTCAGCAGGCTGAGGCAGGAGCATCGCTTGAACCCAGGAGGCAGAGGTTGCAGTGAGCCGAGA
TGGTGCCACTGCACTCCAGCCTGGGATGACAGAGCGAGACTCCATCTCAAAAAAAAAAAAAAAAAAAAAA
TTGAACAGCAAGGTTATCCATATAATATTTCTTTAAAGGGTACAAGAATTTTCCTTTCTGCCTCTAAATA
AAGGATTTCCTAATTCAGTGTGATCCTTAACAGCAACCATGAGGATTACTGAGTGCCTTTCTGGGGCCTT
TTGAATGCTGTTTGGTACAGCACCAGAGTCCCTACTAGATCTAGAGTTGGCTGCTATAGTTTTTTGTGGC
GATTTTTTGCCATGGAGTCATTTGAACCTCATACACAATCCTAACATGCCATCCCCTTTCTGTCATAGCA
GGTACACTAAAATTTCTTTGTAGCTCAATTTTATATAATCAAGATCACATAAATAAGGCTTCCATGTTAG
AATCGTTGCAGTTTTTAGTGTATTCCTTTTTGGAGGCTAAAGTTGTACCTTATAAACTGTTTCTGCGTCT
GGCATTTAGCAAGACAAGTTATTTGGGTTTTCTTTCCCTCCTCTTGAGCTCTCAGCCTTCTGACTACAAG
GTTTGGCTTAAGCCTTATAATCTAAAAAATATCAGCCAGGCTATTCTATCTTCTAAGACCTGGCTGAATC
ATGAGCCAGTTCTAAATCTAAAGAGAGTGAGAGAGGGAAGAAATCTGGCACAAACTTACAGTCTCTTTAA
TTACATGTAAAATGCATGTGACTGTATTACCTATTGGCTTAGCCCCATGGAGGGTTTAGAAAAATGTGTA
GTCTTTGTGGAAGCTATCCAATTATCCTTCTCCCAAAAAGATGTTTTAAATGTGGAATAGTATTACATTC
CCCTGCCCCTTTATGAGTCCTTCATAACTTACTAAAGCTGACCAATTGTTATTTATGTAACCTGGCTCAT
TCATTGTCAACTAAGAACCTAATTATATGCAATTTATTGTAAAAAAAGCTATAAAAATATATTTTGCTAG
TATTTTAGAGGAAAAATGATATTGGGCACAGTCTATAAATGGGGAGAAAAGTTAAGTAGTATCTAGATTC
CAAGGATACTATATTTATTATACAGATATGTGTGCCTGTGCTTCCATCAAACCCTTTTTCAGGTATCTCC
TTTTAATTCATAAGGAGGAAAGAGTAGGGCATTTATAAAGCTAAGCTAAAAATGATGCTAAGCATAACGT
AGATGAGACGCCAGGCTGAACCAGGGGAAGGCTGGCATTGTTAGTGTCCCCAACTAGCAGTCCACCTTTA
TCTGTGGCAGCTATAAATGTACAGGACCCATCAGAGTCCTAAGAAAATGAGAGTAATTATCTCTGGCATC
ATCCACATTTCCGACTCTTTCCAATCTCTTTTCCCTTTTTCTGTAATGTACCCAGCATCCCCCTATTGTA
TTTTGGTTGCCCAAGATTCTTGATTCTTTGAGTGTGTAGTAGCATTTCTTAAAATGAGATCATCAGACCA
ACCCTTGATTCACATGAAAGCTGTAATGACACAACAAAGAGAAGGCGACAGTTTTAAAGTATAATTGTCA
GCCAAATGTGTATTTTATATTTGGTTCATAGAATATATCTAGATGTGGGGAAAGTCTCCTATTTGGTAAT
TTAGTTAAAATGTAAATGTTATATCACAGCATATGTTGGTATGTTTTGGAGTGTGCTTCCATTGTGCTCA
GCTTTTGAAAAGTTTGAAATCCACTTTAGTCAAATGTAGTCAATGGGATTTCCAGAGATACATATTGTTT
TTCTTAGTGTACCACACACTCCTTGAAGGCAGATACTGTACTTAATATATCACTGTCTTCCATAATACTG
CCCTAGGTCTTTTTAGTTTTTAAGAGACCGGGTCTCGCTATGTTTCCCATGCTGAACTCAAATGCCTGGG
CTTAAGCAATCCTCCCACCTCAGCCTCTGGAGTAGCTGGGACTACAGGGGCATGCACCACCAGGCCTGGC
TTCCTAGGAGGGTCTTTAAAGAGAAAATATTTGTTCAATTGAAAACAGGATTCTTGTCATCTACAACTCC
AACACAGCCTGAAAATATCCACATTATAACCTGGACCTTAGACCTACTTTCTCCACTATCCTGCAAAGCT
ACATCTGTAACTACCTATTGGCTATCTATATGAGTCCTCAAGCATCTCAGACTTTACATGAATAAAACTC
AACTTCCTTCCCATTCAAATCTGTTTATTTTCTTCTGTAAGAGAAAGATACCATTTGAGACTCCAGAATC
TGCCTCTAACTCTCAACAAGACTCTGCAATTACTCAAGTATCCTTTCCATCCTCATTGCCCTGCTGTTAT
TACATAGGCCCTGGTTCAAGTCCTTGTTACTTGTTCCCATTATTGCAATAACTTCTAATTCCAATGCCGT
TGTGTGATCCCATTTTAAACACGGCCAGAGCAGTCTTCCAACAACATAGCTCTAATCTAGTTTCATCCCC
ACTTTTACATGCCTCAGTGGCTTTCCCAGTGACTTGGCATGGAACACGTCCTCAGTTGCCATACATTCCA
GCTAACTCTTACCCAACCTTTCTTTGTTCACACAGTTTCCTTTTCCTTCCTCATTGACCCATCCGCATCT
CTGTTTATCCAAGACTTCTCTGTGATAGCTGACCCTTAGTCTTTCTCTCCCCTATTCCTCCAGACTAGAT
CCTGTCTCCTTCCTGCAGCCCCGACACAGCCTTCAGTTCATATCTTTTGCATGATGCTTAGCACCTTCTA
TCCCTAAGGACAACTTACTCATTTGAGATTTCTGGCAGGGTACCTTGCATGCAGTGGACACTCAGTATTT
GCTGAATTAAATTCCTTCCTATGGATCCCTTCTGATTTTTTTTAAGTGCCTCTAATACACATATCATTCT
AGGGCTCATGCCACTTTTAATGTCATTTTCTAAAGGAAAATCTTATCTATGATATTTTCCCTTATAAGAG
ATAGTTGTTTTGAGTAGGGTTTTTTAAAAGATAAAGGTAGTAGGAAATTTTTTAAAGCCTAAATATCAAA
TTCCTTTCCCTTTGGAGTTGGGGGAAGGAATGAAGGGGGAGCAACTTGCTCTTTCATATGAGTTGGTCAT
AGCATGTAAGAACCAATCTTGAAATATCGTTTTTTTTTTAATGGCTTATAATGTATTTCTAGAAATACTT
TGTACTTAAAATGATAACAGTTTGTATCTTTTTGTCCATATATACTTTATAAATAAAAAAATTAGCATTG
TAAATAATGTTAATATGTATTTATACAAAATAAATTTACTATAATATA

By “vezatin, adherens junctions transmembrane protein (VEZT) nucleic acid molecule” is meant a polynucleotide encoding a VEZT polypeptide, as summarized in NCBI Gene ID 55591. An exemplary VEZT nucleic acid molecule is provided at NCBI Accession No. NM_{—}017599.3, as well as below:

>gi|155030243|ref|NM_017599.3| Homo sapiens
vezatin, adherens junctions transmembrane protein
(VEZT), transcript variant 1, mRNA
GTAGTTTTCTGGACCCACGGGACGGGCAGGAGCTGGAGCTCCGTGCCGC
CTGTACTCCCGCCTTCATTTCCCATCGTGCTGAGGCGGGTGGCATGGCG
GAGAAGGATGACACCGGAGTTTGACGAAGAGGTGGTTTTTGAGAATTCT
CCACTTTACCAATACTTACAGGATCTGGGACACACAGACTTTGAAATAT
GTTCTTCTTTGTCACCAAAAACAGAAAAATGCACAACAGAGGGACAACA
AAAGCCTCCTACAAGAGTCCTACCAAAACAAGGTATCCTGTTAAAAGTG
GCTGAAACCATCAAAAGTTGGATTTTTTTTTCTCAGTGCAATAAGAAAG
ATGACTTACTTCACAAGTTGGATATTGGATTCCGACTCGACTCATTACA
TACCATCCTGCAACAGGAAGTCCTGTTACAAGAGGATGTGGAGCTGATT
GAGCTACTTGATCCCAGTATCCTGTCTGCAGGGCAATCTCAACAACAGG
AAAATGGACACCTTCCAACACTTTGCTCCCTGGCAACCCCTAATATTTG
GGATCTCTCAATGCTATTTGCCTTCATTAGCTTGCTCGTTATGCTTCCC
ACTTGGTGGATTGTGTCTTCCTGGCTGGTATGGGGAGTGATTCTATTTG
TGTATCTGGTCATAAGAGCTTTGAGATTATGGAGGACAGCCAAACTACA
AGTGACCCTAAAAAAATACAGCGTTCATTTGGAAGATATGGCCACAAAC
AGCCGAGCTTTTACTAACCTCGTGAGAAAAGCTTTACGTCTCATTCAAG
AAACCGAAGTGATTTCCAGAGGATTTACACTGGTCAGTGCTGCTTGCCC
ATTTAATAAAGCTGGACAGCATCCAAGTCAGCATCTCATCGGTCTTCGG
AAAGCTGTCTACCGAACTCTAAGAGCCAACTTCCAAGCAGCAAGGCTAG
CTACCCTATATATGCTGAAAAACTACCCCCTGAACTCTGAGAGTGACAA
TGTAACCAACTACATCTGTGTGGTGCCTTTTAAAGAGCTGGGCCTTGGA
CTTAGTGAAGAGCAGATTTCAGAAGAGGAAGCACATAACTTTACAGATG
GCTTCAGCCTGCCTGCATTGAAGGTTTTGTTCCAACTCTGGGTGGCACA
GAGTTCAGAGTTCTTCAGACGGTTAGCCCTATTACTTTCTACAGCCAAT
TCACCTCCTGGGCCCTTACTTACTCCAGCACTTCTGCCTCATCGTATCT
TATCTGATGTGACTCAAGGTCTACCTCATGCTCATTCTGCCTGTTTGGA
AGAGCTTAAGCGCAGCTATGAGTTCTATCGGTACTTTGAAACTCAGCAC
CAGTCAGTACCGCAGTGTTTATCCAAAACTCAACAGAAGTCAAGAGAAC
TGAATAATGTTCACACAGCAGTGCGTAGCTTGCAGCTCCATCTGAAAGC
ATTACTGAATGAGGTAATAATTCTTGAAGATGAACTTGAAAAGCTTGTT
TGTACTAAAGAAACACAAGAACTAGTGTCAGAGGCTTATCCCATCCTAG
AACAGAAATTAAAGTTGATTCAGCCCCACGTTCAAGCAAGCAACAATTG
CTGGGAAGAGGCCATTTCTCAGGTCGACAAACTGCTACGAAGAAATACA
GATAAAAAAGGCAAGCCTGAAATAGCATGTGAAAACCCACATTGTACAG
TAGTACCTTTGAAGCAGCCTACTCTACACATTGCAGACAAAGATCCAAT
CCCAGAGGAGCAGGAATTAGAAGCTTATGTAGATGATATAGATATTGAT
AGTGATTTCAGAAAGGATGATTTTTATTACTTGTCTCAAGAAGACAAAG
AGAGACAGAAGCGTGAGCATGAAGAATCCAAGAGGGTGCTCCAAGAATT
AAAATCTGTGCTGGGATTTAAAGCTTCAGAGGCAGAAAGGCAGAAGTGG
AAGCAACTTCTATTTAGTGATCATGCCGTGTTGAAATCCTTGTCTCCTG
TAGACCCAGTGGAACCCATAAGTAATTCAGAACCATCAATGAATTCAGA
TATGGGAAAAGTCAGTAAAAATGATACTGAAGAGGAAAGTAATAAATCC
GCCACAACAGACAATGAAATAAGTAGGACTGAGTATTTATGTGAAAACT
CTCTAGAAGGTAAAAATAAAGATAATTCTTCAAATGAAGTCTTCCCCCA
AGGAGCAGAAGAAAGAATGTGTTACCAATGTGAGAGTGAAGATGAACCA
CAAGCAGATGGAAGTGGTCTGACCACTGCCCCTCCAACTCCCAGGGACT
CATTACAGCCCTCCATTAAGCAGAGGCTGGCACGGCTACAGCTGTCACC
AGATTTTACCTTCACTGCTGGCCTTGCTGCAGAAGTGGCTGCTAGATCT
CTCTCCTTTACCACCATGCAGGAACAGACTTTTGGTGGTGAGGAGGAAG
AACAAATAATAGAAGAAAATAAAAATGAGATAGAAGAAAAGTAAGAACC
AAGATTCATATGAAGTGATATTAGATTGTTCCTTTTACAAAAGTGTTTA
GCTTCAAGACTGGAAAGGGAATATGAGTGTAAGTTTACTATATATAAAG
CTAAGATGTGGATTTACAGGAAGAACCCTGGTTTGAATAACTGATCTGA
AATTAGTAGTTACCTGTAAATGGCAGATCTTTTAGGAAAATAAGAGAAA
GGTAAGGGCTCTTTTGAATAAACTGCTGTTTTATTTGTGGCACAACTGA
TCAATCTTGGAAATTCTTTAAGTATTTTTAATAAGAAATGAATTATCAT
TTCTTGCCAGAATTTGCTACCTTAAGGTGATTGGGAAAATTCTGTTGCA
AGAACATTAACATTTAGTATGACTCCTTTTTACTGTATTCTTGCAGTTA
ATAACTGCAGCTATTATGTTAATAACAAGTTGTTTGTATTTTATTTTTG
TTTATACCAGTCTTAAAGATCCAGGTTCTGAATAAAAAAATTAATTGAT
ACAATTGATGTGTGCTGGGGTTTGGAACTAAAAGTAGTTTCAACAGTGC
GTGGGTTATGACATTTCTTATGTTTCTTTGTTCATGTGTGTATTTAGTA
GTTAATTTTAAGATGTCCTAGTGATCTTTAAAAGAAAAATATTGTACCA
TTTTTTAGAATTACACTTTCACCTTTCTTTTTGCAATTGAAAGTGATGA
TGTCAAAGTGGGATTTCTGTACTCCAAGGCCCCACCCCCAATTTAGCAA
GCAGAAAAACGTTCCTTGTATCACTTTACCTTGGATAATTGGGTGCCAT
TAACACAAACAGGTCACAATCCTGCTGTTTTCTAGCCCTGTCCACCATA
ATGAGATTCAGGAAACATCCTGTCAGCCTCCTGGAAAGCATCCTTGTCT
CCTTAGTATTTCATTTACAAACTACCTCTTAACAGAGACTGCTTTTCAA
ATTGGCCAATCTTACCTGTTTTGTGTTGTGATTGCATTTTCAAAGAGTA
ATTATTTTCAGCATATACAGTTTTGAAACCTGTAGCTCCTATGCAATAA
CATAGTTCTATAGACATTATTTGGGGGAAATGTAGTAATAACTCAATCT
ATGTTGCTGTCCTAGAAAGGAAATTGCATGATGAATCTAGATTGTCTTT
AGAGTAAAGAAACACATTCAAATTCCTGTAACTTATCACTTTCAGTGAG
TAAATTTACTTATACCAAAGGGGATTTTTTTTCTTTCAGGAATCTAAGG
AAATTTACTTTTTAACCTGAGAAAAAAACTTGGTTCTGCTTTATATAAA
CAGTAGAGATTATTGTACTATAAGTGATTTTGCCTTTTTGCCAAAATCC
TGGAACTCATCTATAATTAACCTCTTCGGAGCAATACCTTAGGTTGGGC
CTTGCTTTACTACTTAGAAATAGCTAAATTTCAATTTTAAAAATCTTTG
TGTGTTATAACTGTTAAATTATTCAATAATACTTAGGGTTTACTTTCTT
ATTTAAATCACTTATTTAGTTTACCGACTTCATTTTTCTTTGGATTTAG
AAGAAGCAATTATGGAAAAACTTGGTAATCTCTCTCAACCTATAACCTT
ACACAGGAAGAATTAGAGTTTAATAATTTTTAATTCTTTTATTGTATGT
TACTTTTATTACACCAGTTTGGGGGAAAATCTTCATAAAATTGTATCAG
TTTTATTCAGTGTTCTCTAAGGTGATACCTTTTAATTTTGAAAGACTAA
ATAATTTTAATCGAGAATTTCCAGTCTTTCAGTCTGATCTATTTAATTC
ACTACTTGTTACATAATCCAGTGAAAACTCTACTTGTTGAAATTATGAC
ATAAAGATCTTGCAGCTTTATTTGAGTATTTGTTCTTTTGTGTAGTTTC
CATCTTTTAAAATATTTAAAATATTTTCAAGATAAAGTATTATCTTCTC
TGCAAAAATTCCTGGAGTAATTTTCTCTCATAATATTTGAAGTCAGTGG
TTCTCAGTTGTATTAGTGGGGTAACTACATCAAAATAAATAAAGTCTTA
TTTTTAAAATGCAAATTTTAGACCATACTCCCAGTGATTCTTAGTTGGT
CTTTTTGGAATGAGCCATAGGTAATGTTTATGTCCAATAAAATCTAGGA
ACCTCAAAAAAAAAAAAAAAAAA

By “growth differentiation factor 3 (GDF3) nucleic acid molecule” is meant a polynucleotide encoding a GDF3 polypeptide, and as summarized in NCBI Gene ID 9573. An exemplary GDF3 nucleic acid molecule is provided at NCBI Accession No. NM_{—}020634.1, as well as below:

>gi|10190669|ref|NM_020634.1| Homo sapiens
growth differentiation factor 3 (GDF3), mRNA
GGAGCTCTCCCCGGTCTGACAGCCACTCCAGAGGCCATGCTTCGTTTCT
TGCCAGATTTGGCTTTCAGCTTCCTGTTAATTCTGGCTTTGGGCCAGGC
AGTCCAATTTCAAGAATATGTCTTTCTCCAATTTCTGGGCTTAGATAAG
GCGCCTTCACCCCAGAAGTTCCAACCTGTGCCTTATATCTTGAAGAAAA
TTTTCCAGGATCGCGAGGCAGCAGCGACCACTGGGGTCTCCCGAGACTT
ATGCTACGTAAAGGAGCTGGGCGTCCGCGGGAATGTACTTCGCTTTCTC
CCAGACCAAGGTTTCTTTCTTTACCCAAAGAAAATTTCCCAAGCTTCCT
CCTGCCTGCAGAAGCTCCTCTACTTTAACCTGTCTGCCATCAAAGAAAG
GGAACAGTTGACATTGGCCCAGCTGGGCCTGGACTTGGGGCCCAATTCT
TACTATAACCTGGGACCAGAGCTGGAACTGGCTCTGTTCCTGGTTCAGG
AGCCTCATGTGTGGGGCCAGACCACCCCTAAGCCAGGTAAAATGTTTGT
GTTGCGGTCAGTCCCATGGCCACAAGGTGCTGTTCACTTCAACCTGCTG
GATGTAGCTAAGGATTGGAATGACAACCCCCGGAAAAATTTCGGGTTAT
TCCTGGAGATACTGGTCAAAGAAGATAGAGACTCAGGGGTGAATTTTCA
GCCTGAAGACACCTGTGCCAGACTAAGATGCTCCCTTCATGCTTCCCTG
CTGGTGGTGACTCTCAACCCTGATCAGTGCCACCCTTCTCGGAAAAGGA
GAGCAGCCATCCCTGTCCCCAAGCTTTCTTGTAAGAACCTCTGCCACCG
TCACCAGCTATTCATTAACTTCCGGGACCTGGGTTGGCACAAGTGGATC
ATTGCCCCCAAGGGGTTCATGGCAAATTACTGCCATGGAGAGTGTCCCT
TCTCACTGACCATCTCTCTCAACAGCTCCAATTATGCTTTCATGCAAGC
CCTGATGCATGCCGTTGACCCAGAGATCCCCCAGGCTGTGTGTATCCCC
ACCAAGCTGTCTCCCATTTCCATGCTCTACCAGGACAATAATGACAATG
TCATTCTACGACATTATGAAGACATGGTAGTCGATGAATGTGGGTGTGG
GTAGGATGTCAGAAATGGGAATAGAAGGAGTGTTCTTAGGGTAAATCTT
TTAATAAAACTACCTATCTGGTTTATGACCACTTAGATCGAAATGTCA

By “microRNA 331 (MIR331) nucleic acid molecule” is meant a polynucleotide encoding a microRNA. An exemplary MIR331 nucleic acid molecule is provided at NCBI Accession No. NR_{—}029895.1, as well as below:

GAGTTTGGTTTTGTTTGGGTTTGTTCTAGGTATGGTCCCAGGGATCCCA
GATCAAACCAGGCCCCTGGGCCTATCCTAGAACCAACCTAAGCTC

By “ribosomal protein L29 pseudogene 26 (RPL29P26) nucleic acid molecule” is meant a polynucleotide encoding a RPL29P26 pseudogene. An exemplary RPL29P26 nucleic acid molecule is provided at NCBI Accession No. gi1224589803:c95861652-95861038, as well as below:

GCTTAAGGTGCAGACATGGCCAAGTCCAAGAACCACACCACACACAACC
AGTCCTGAAAATGGCACAGAAATGGTATCAAGAAACCCCGATCACAAAG
ATACGAATCTCTTAAGGGGGTGGACCCCAAGTTCCTGAGGAACATGCGC
TTTGCCAAGAAGCACAACAAGAAGGGCCTAAAGAAGATGCAGGCCAACA
ATGCCAAGGCCATGAGTGCACGTGCCGAGGCTATCAAGGCCCTCGTAAA
GCCCAAGGAGGTTAAGCCCAAGATCCCAAAGGGTGTCAGCCACAAGCTC
GATTGACTTGCCTACATTGCCCACCCCAAGCTTGGGAAGCGTGCTTGTG
CCCATATTGCCAAAGGGCTCAGGCTGTGCCGGCCAAAGGCCAAGGCCAA
GGATCAAACCAAGGCCCAGGCTGCAGCTCCAGCTTCAGTTCCAGCTCAG
GCTCCCAAAGGTGCCCAGGCCCCTACAAAGGCTTCAGAGTAGATATCTC
TGCCAATGTGAGGACAGAAGGACTGGTGCGACCCCCCACCCCCGCCCCT
GGGCTACCATCTGCATGGGGCTGGGGTCCTCCTGTGCTATTTGTACAAA
TAAACCTGAGGCAGGAAAAAAAAAAAA

By “hypothetical protein LOC729457 (LOC729457) nucleic acid molecule” is meant a polynucleotide encoding a hypothetical LOC729457 polypeptide. An exemplary LOC729457 nucleic acid molecule is provided at NCBI Accession No. gi189161190:c32151164-32150334, as well as below:

ATGTCTCCCGGGCCGCGTCACTGCAGTCTCGCCCTGGGTCTGGCGCGCT
CCGGCTCGCGGCTCGCTCTCTCGCTCCACCTGCTCCCTCTGGCCCTGCA
GCAGCCGGTGCGGAATGATGCAGTCTCGGGGCCGGCTCCCTCCCTTCCC
GCGTGGCGGCGGCTCCGAGCAGGGGGCGGGGAGCGGATGGAGTCAGCGC
GGGGGGCGGAGGGAAGGACCAGACGGAAACATCCCGAGGCGCCTCCCGC
CGGGCGCGCGGGCCGCCGCCCGCTGCACCGTGAGGCGCGCCAGGAGGAG
GCGCAGGCGACGGGTCTGGGACTGGGAAGCGGTGGGGCGCGCGCGGCGG
GGGAGCCTCCGCCCTGTCCGGCTCGCGGGGGCGGGAGCTCCTCCCAGGG
CTTTGTCCCGGTGGCAGTAGAAGACCCCGAGAGCGGCGTGGGCGCCCGG
GCTCTTTTGCTACGTCGAGGGCCGAAGCTCAGGAAACTGCCTGGAACGC
TTTCTCCCGAGAAAAGCAAACAAAACTATCGCGGTCGCGGTCCGCGCAT
CCTCCTCGTCCCCTGGGCGCGCAGAAGGCTTTTTGGGCCACCTGCCCCC
AAAAGACCGCTGGGTTTCCCAAAGCTTTCAAGACGCACCCCAAGGCGCC
CTCCTCCGTCGTCCCCCTCTCTCCCTGCCTCTCCCAAGTCTGGCCTGGG
CCACCTAACACTCTCACCAGATAACCTTACTATCCTCACAGGACAGTCC
GCTAAATATTGCTCGCCCTCACCCAGCGTATCACAAGAGCGCTATCCAC
TCAGAAAAAAAATATCTCCACAATACATGCACCCAGGAAACCTCTAG

By “methionyl aminopeptidase 2 (METAP2) nucleic acid molecule” is meant a polynucleotide encoding a METAP2polypeptide. An exemplary METAP2nucleic acid molecule is provided at NCBI Accession No. NM_{—}006838.3, as well as below:

GAGTCCTCCGCCGTCCCAGCATTCCCTGCGTCCCTACCATCGAGAGCAG
CTTCCGGCGTGGCTGGTGTAGGCGGGTGGAGAAGGATCGGGGCCCTCGC
CGCTCTGTCTCATTCCCTCGCGCTCTCTCGGGCAACATGGCGGGTGTGG
AGGAGGTAGCGGCCTCCGGGAGCCACCTGAATGGCGACCTGGATCCAGA
CGACAGGGAAGAAGGAGCTGCCTCTACGGCTGAGGAAGCAGCCAAGAAA
AAAAGACGAAAGAAGAAGAAGAGCAAAGGGCCTTCTGCAGCAGGGGAAC
AGGAACCTGATAAAGAATCAGGAGCCTCAGTGGATGAAGTAGCAAGACA
GTTGGAAAGATCAGCATTGGAAGATAAAGAAAGAGATGAAGATGATGAA
GATGGAGATGGCGATGGAGATGGAGCAACTGGAAAGAAGAAGAAAAAGA
AGAAGAAGAAGAGAGGACCAAAAGTTCAAACAGACCCTCCCTCAGTTCC
AATATGTGACCTGTATCCTAATGGTGTATTTCCCAAAGGACAAGAATGC
GAATACCCACCCACACAAGATGGGCGAACAGCTGCTTGGAGAACTACAA
GTGAAGAAAAGAAAGCATTAGATCAGGCAAGTGAAGAGATTTGGAATGA
TTTTCGAGAAGCTGCAGAAGCACATCGACAAGTTAGAAAATACGTAATG
AGCTGGATCAAGCCTGGGATGACAATGATAGAAATCTGTGAAAAGTTGG
AAGACTGTTCACGCAAGTTAATAAAAGAGAATGGATTAAATGCAGGCCT
GGCATTTCCTACTGGATGTTCTCTCAATAATTGTGCTGCCCATTATACT
CCCAATGCCGGTGACACAACAGTATTACAGTATGATGACATCTGTAAAA
TAGACTTTGGAACACATATAAGTGGTAGGATTATTGACTGTGCTTTTAC
TGTCACTTTTAATCCCAAATATGATACGTTATTAAAAGCTGTAAAAGAT
GCTACTAACACTGGAATAAAGTGTGCTGGAATTGATGTTCGTCTGTGTG
ATGTTGGTGAGGCCATCCAAGAAGTTATGGAGTCCTATGAAGTTGAAAT
AGATGGGAAGACATATCAAGTGAAACCAATCCGTAATCTAAATGGACAT
TCAATTGGGCAATATAGAATACATGCTGGAAAAACAGTGCCGATTGTGA
AAGGAGGGGAGGCAACAAGAATGGAGGAAGGAGAAGTATATGCAATTGA
AACCTTTGGTAGTACAGGAAAAGGTGTTGTTCATGATGATATGGAATGT
TCACATTACATGAAAAATTTTGATGTTGGACATGTGCCAATAAGGCTTC
CAAGAACAAAACACTTGTTAAATGTCATCAATGAAAACTTTGGAACCCT
TGCCTTCTGCCGCAGATGGCTGGATCGCTTGGGAGAAAGTAAATACTTG
ATGGCTCTGAAGAATCTGTGTGACTTGGGCATTGTAGATCCATATCCAC
CATTATGTGACATTAAAGGATCATATACAGCGCAATTTGAACATACCAT
CCTGTTGCGTCCAACATGTAAAGAAGTTGTCAGCAGAGGAGATGACTAT
TAAACTTAGTCCAAAGCCACCTCAACACCTTTATTTTCTGAGCTTTGTT
GGAAAACATGATACCAGAATTAATTTGCCACATGTTGTCTGTTTTAACA
GTGGACCCATGTAATACTTTTATCCATGTTTAAAAAAGAAGGAATTTGG
ACAAAGGCAAACCGTCTAATGTAATTAACCAACGAAAAAGCTTTCCGGA
CTTTTAAATGCTAACTGTTTTTCCCCTTCCTGTCTAGGAAAATGCTATA
AAGCTCAAATTAGTTAGGAATGACTTATACGTTTTGTTTTGAATACCTA
AGAGATACTTTTTGGATATTTATATTGCCATATTCTTACTTGAATGCTT
TGAATGACTACATCCAGTTCTGCACCTATACCCTCTGGTGTTGCTTTTT
AACCTTCCTGGAATCCATTTTCTAAAAAATAAAGACATTTTCAGATCTG
AGAGCTACATCTCAATGTCTGTGGTTATAATTCTGGACAGGATAAATAG
CTAAACTTAATGTAGGCAAATGCAGAGACATTTATCTGAAATGTAGACC
TCTACACTGAGACTTTTCTGGCATAGTGGCTAAAACAAGATCTACACAT
GCATAAAAAGGGACAATCACCTTTTCTTCATAAATATACAGCTTTAGGA
ATATTTCACCATTCTTTGTAGGACATAGTAGTCCTTGTCTTTTTTTCTC
CTGACATTGGAAAGATGTGCTAATTGAAACTTGACTTAGTAGGAACATT
GTGCCAACTCAAAACCTTGATTTAGTAAAAATCTCAATGTTTAGATCCT
TTGTCCAGTGGTGGTGTTTATCAGGGAATGTATTCAGCTTGCTCAGAAA
ACCAAAAGGGTATTAAAGCCACAAAAGCAAAGAAGAAAAAAAAAAACTT
CCCATGTTTGGATCTTGTTCTAGTTAGAAAAATTAAGTTGAAATTCTTG
GACTTTTTCATTCATGAGGCAAATGCTGTAATACCTTCCCCTTTGACAG
GTTTGGATTCTTAACATTACTAGTGGTATTTCAGGAAGTGACGTTACAG
TTACTTTCCTTATAGCGGCTAAGTGTATTAAGTTGAATGTAACGATGGT
AATATTAATTTGTTTGAACTGAGGCCCACTACTGATTCTTTGACAAATT
GAATTCTTATATTTAAATAATTTTATGGGAATGTTCCATCATAATTTCT
AAATCATTTATATATCAAGGTAGCCTTAATTTGTATATGTTTCAGTACA
ATGAGATTTTATTGCCTCTGGGATGCTGTTTAGTTTGTATTTTGTTGAA
CGTTTTTATCCTAGGAAGAGAAACCTATGACTTGTGTACCTAGATCATC
TGTTACATTAAAAAGCTGCTCTTTCAGCATTAGAGCTATAAATGAATGT
TACCTTGTCGGGAAACAATCTAGGTTTTAGCTGTATGAGCTATGTTTAT
TATGGTGCTAATGTTCAGTAGCCACATTTGACTAATGTCTCCATTCTCT
GTGATGCTGTGGCTAGCAGCAGAGCTCGCCAGTTCATGCCTGGACATAC
TGTCAGGGCTGGGCCCTCCAGCTAGCTCCTTTGGGGTTGAGTCCGTATC
TTTTTGATGTGGAAGTATAAAGCAAGTATCTTGATTTCTAAACCCAGCA
ATTTTAGAATTGACCTTTATGAGTGAAGACTTTTGGAGCTTTTAAAGAC
CTTGGCAGTCATGATCTCAAACCAATTAGGAGCTCCAAGCTCCCTTCCC
AGGTAACTGTTGGGAGCAATGGCATCACTGTATGCCCTTGTAATGGCTG
GAAGGGACATGATCTTGTAAGTAGGAAAGCTGTAACTAAAAATTGTATT
GTTTGCTTATTAGCCATGTATCTCTTAAAATTTTGTTATGTTTACAACG
ATGTACCTTATTGGCAACAAGTTATTAGTTTGATGTTTAACAATAGTGC
CTTTAGTAAATTATTTTACAACTAAAA

By “ubiquitin specific peptidase 44 (USP44) nucleic acid molecule” is meant a polynucleotide encoding a USP44polypeptide. An exemplary USP44 nucleic acid molecule is provided at NCBI Accession No. NM_{—}001042403.1, as well as below:

GGGTCGTCGCGGCCGCCGAACCGGGGGGCGGGGGGCCGGGGTGAGCGCT
AAGATGGCCGCCCCGGCTCGGGCTGTTTTCAGATGCTTCAAGTGTTGTG
AACAGAGACTTGTTTGGATTATGCATTTCTCAGCTAGACTAAATAAATG
CTAGCAATGGATACGTGCAAACATGTTGGGCAGCTGCAGCTTGCTCAAG
ACCATTCCAGCCTCAACCCTCAGAAATGGCACTGTGTGGACTGCAACAC
GACCGAGTCCATTTGGGCTTGCCTTAGCTGCTCCCATGTTGCCTGTGGA
AGATATATTGAAGAGCATGCACTCAAGCACTTTCAAGAAAGCAGTCATC
CTGTTGCATTGGAGGTGAATGAGATGTACGTTTTTTGTTACCTTTGTGA
TGATTATGTTCTGAATGATAACACAACTGGAGACCTGAAGTTACTACGA
CGTACATTAAGTGCCATCAAAAGTCAAAATTATCACTGCACAACTCGTA
GTGGGAGGTTTTTACGGTCCATGGGTACAGGTGATGATTCTTATTTCTT
ACATGACGGTGCCCAATCTCTGCTTCAAAGTGAAGATCAACTGTATACT
GCTCTTTGGCACAGGAGAAGGATACTAATGGGTAAAATCTTTCGAACAT
GGTTTGAACAATCACCCATTGGAAGAAAAAAGCAAGAAGAACCATTTCA
GGAAAAAATAGTAGTAAAAAGAGAAGTAAAGAAAAGACGGCAGGAATTG
GAGTATCAAGTTAAAGCAGAATTGGAAAGTATGCCTCCAAGAAAGAGTT
TACGTTTACAAGGGCTCGCTCAGTCGACCATAATAGAAATAGTTTCTGT
TCAGGTGCCAGCACAAACGCCAGCATCACCAGCAAAAGATAAAGTACTC
TCTACCTCAGAAAATGAAATATCTCAAAAAGTCAGTGACTCCTCAGTTA
AACGAAGGCCAATAGTAACTCCTGGTGTAACAGGATTGAGAAATTTGGG
AAATACTTGCTATATGAATTCTGTTCTTCAGGTGTTGAGTCATTTACTT
ATTTTTCGACAATGTTTTTTAAAGCTTGATCTGAACCAATGGCTGGCTA
TGACTGCTAGCGAGAAGACAAGATCTTGTAAGCATCCACCAGTCACAGA
TACAGTAGTATATCAAATGAATGAATGTCAGGAAAAAGATACAGGTTTT
GTTTGCTCCAGACAATCAAGTCTGTCATCAGGACTAAGTGGTGGAGCAT
CAAAAGGTAGAAAGATGGAACTTATTCAGCCAAAGGAGCCAACTTCACA
GTACATTTCTCTTTGTCATGAATTGCATACTTTGTTCCAAGTCATGTGG
TCTGGAAAGTGGGCGTTGGTCTCACCATTTGCTATGCTACACTCAGTGT
GGAGACTCATTCCTGCCTTTCGTGGTTACGCCCAACAAGACGCTCAGGA
ATTTCTTTGTGAACTTTTAGATAAAATACAACGTGAATTAGAGACAACT
GGTACCAGTTTACCAGCTCTTATCCCCACTTCTCAAAGGAAACTCATCA
AACAAGTTCTGAATGTTGTAAATAACATTTTTCATGGACAACTTCTTAG
TCAGGTTACATGTCTTGCATGTGACAACAAATCAAATACCATAGAACCT
TTCTGGGACTTGTCATTGGAGTTTCCAGAAAGGTATCAATGCAGTGGAA
AAGATATTGCTTCCCAGCCATGTCTGGTTACTGAAATGTTGGCCAAATT
TACAGAAACTGAAGCTTTAGAAGGAAAAATCTACGTATGTGACCAGTGT
AACTCAAAGCGTAGAAGGTTTTCCTCCAAACCAGTTGTACTCACAGAAG
CCCAGAAACAACTTATGATATGCCACCTACCTCAGGTTCTCAGACTGCA
CCTCAAACGATTCAGGTGGTCAGGACGTAATAACCGAGAGAAGATTGGT
GTTCATGTTGGCTTTGAGGAAATCTTAAACATGGAGCCCTATTGCTGCA
GGGAGACCCTGAAATCCCTCAGACCAGAATGCTTTATCTATGACTTGTC
CGCGGTGGTGATGCACCATGGGAAAGGATTTGGCTCAGGGCACTACACT
GCCTACTGCTATAATTCTGAAGGAGGGTTCTGGGTACACTGCAATGATT
CCAAACTAAGCATGTGCACTATGGATGAAGTATGCAAGGCTCAAGCTTA
TATCTTGTTTTATACCCAACGAGTTACTGAGAATGGACATTCTAAACTT
TTGCCTCCAGAGCTCCTGTTGGGGAGCCAACATCCCAATGAAGACGCTG
ATACCTCGTCTAATGAAATCCTTAGCTGATCCAAAGACAATGGGGTTTT
CTTCCTGTGATTTATATATATACTTTTTAAAAGACTGATGTACCATTTT
AAACTTCATTTTTTCTTGTGAATCAGTGTATACTACATTTATACATTTT
ATATCTAACAATTTTTTTTTTTACAAAGTATAAATGTATATATCAACTG
AAGGTAACTACTTTTTTCATATTTGGAGTTTTAAACTTTTGGTGTTTAC
CTCAGACTGATGTTACCTCTTTTATATTTTTATGTCTTAATTGGCTCGG
ATGATGAACTTGTGCAATCTTCTACCAACAAAGTTCAAGTGGCATCATT
TTATATACATGTATCTTTTTCAGGTATTTTCTATACAAATTCTTAATAG
ATGGAAAATTAGACTCTACTTTGGTCACTAATAGTCTTTCATTTGTATA
TTGAAGTTACCTTGCCCCTTGGAGTTATTGAAGTGACATGTCAAGGTAT
CACCTAAATATTCTTCAGTCACACTCACTGGTATTTCTGAGGCTTTGTG
TGTTAACAGGCCTTGTAATTGACATTATTTTGGTTAATGTAACCCCAAA
ATTGCTTTAGTAATTGCTCTTTGGCATAGTCAAACTATAAATGAAAATG
GCAGCTTTACAAATAGTATATTTAAGTGAACTCTGGAACTATGGACATG
AAAAAAATGATGGCTGGGATTTATGATTTTTGTCTGGCAGCAAACAGGT
TTGTCCAGAAGTCTAATAATTAAGCAGTCATAAAAAGTCTGAATTTAGT
AAACCAGTGTATGATGTTATTCAAATAGTTTACCTTGGGTATGAGTTCA
TTTTATAATGTCTGATGACATTAGATCTCTTAAAACTTTATGTATTTTT
TTTAGTTCAAAGGAATAGAGTCTTGAAGAGAAAAAATTATAGGGCAGAA
AAGATAAGTGTTCAAAATTGGCAACTGGACTATTATTATGTCTAGCATC
TCATTCTAAATAACTAAAGCTTGATTTACTCTTGCTAGGATTATGTGAC
TACTAGGTAGGAGCCTCTTAAAACACTGGCCCTGAGCATTAAAAAAAAA
AA

By “CD163 molecule-like 1 (CD163L1) nucleic acid molecule” is meant a polynucleotide encoding a CD163L1polypeptide. An exemplary CD163Llnucleic acid molecule is provided at NCBI Accession No. NM_{—}174941.4, as well as below:

AGGACTCAGGAAGAGATAGACCCATAATGATGCTGCCTCAAAACTCGTG
GCATATTGATTTTGGAAGATGCTGCTGTCATCAGAACCTTTTCTCTGCT
GTGGTAACTTGCATCCTGCTCCTGAATTCCTGCTTTCTCATCAGCAGTT
TTAATGGAACAGATTTGGAGTTGAGGCTGGTCAATGGAGACGGTCCCTG
CTCTGGGACAGTGGAGGTGAAATTCCAGGGACAGTGGGGGACTGTGTGT
GATGATGGGTGGAACACTACTGCCTCAACTGTCGTGTGCAAACAGCTTG
GATGTCCATTTTCTTTCGCCATGTTTCGTTTTGGACAAGCCGTGACTAG
ACATGGAAAAATTTGGCTTGATGATGTTTCCTGTTATGGAAATGAGTCA
GCTCTCTGGGAATGTCAACACCGGGAATGGGGAAGCCATAACTGTTATC
ATGGAGAAGATGTTGGTGTGAACTGTTATGGTGAAGCCAATCTGGGTTT
GAGGCTAGTGGATGGAAACAACTCCTGTTCAGGGAGAGTGGAGGTGAAA
TTCCAAGAAAGGTGGGGAACTATATGTGATGATGGGTGGAACTTGAATA
CTGCTGCCGTGGTGTGCAGGCAACTAGGATGTCCATCTTCTTTTATTTC
TTCTGGAGTTGTTAATAGCCCTGCTGTATTGCGCCCCATTTGGCTGGAT
GACATTTTATGCCAGGGGAATGAGTTGGCACTCTGGAATTGCAGACATC
GTGGATGGGGAAATCATGACTGCAGTCACAATGAGGATGTCACATTAAC
TTGTTATGATAGTAGTGATCTTGAACTAAGGCTTGTAGGTGGAACTAAC
CGCTGTATGGGGAGAGTAGAGCTGAAAATCCAAGGAAGGTGGGGGACCG
TATGCCACCATAAGTGGAACAATGCTGCAGCTGATGTCGTATGCAAGCA
GTTGGGATGTGGAACCGCACTTCACTTCGCTGGCTTGCCTCATTTGCAG
TCAGGGTCTGATGTTGTATGGCTTGATGGTGTCTCCTGCTCCGGTAATG
AATCTTTTCTTTGGGACTGCAGACATTCCGGAACCGTCAATTTTGACTG
TCTTCATCAAAACGATGTGTCTGTGATCTGCTCAGATGGAGCAGATTTG
GAACTGCGACTAGCAGATGGAAGTAACAATTGTTCAGGGAGAGTAGAGG
TGAGAATTCATGAACAGTGGTGGACAATATGTGACCAGAACTGGAAGAA
TGAACAAGCCCTTGTGGTTTGTAAGCAGCTAGGATGTCCGTTCAGCGTC
TTTGGCAGTCGTCGTGCTAAACCTAGTAATGAAGCTAGAGACATTTGGA
TAAACAGCATATCTTGCACTGGGAATGAGTCAGCTCTCTGGGACTGCAC
ATATGATGGAAAAGCAAAGCGAACATGCTTCCGAAGATCAGATGCTGGA
GTAATTTGTTCTGATAAGGCAGATCTGGACCTAAGGCTTGTCGGGGCTC
ATAGCCCCTGTTATGGGAGATTGGAGGTGAAATACCAAGGAGAGTGGGG
GACTGTGTGTCATGACAGATGGAGCACAAGGAATGCAGCTGTTGTGTGT
AAACAATTGGGATGTGGAAAGCCTATGCATGTGTTTGGTATGACCTATT
TTAAAGAAGCATCAGGACCTATTTGGCTGGATGACGTTTCTTGCATTGG
AAATGAGTCAAATATCTGGGACTGTGAACACAGTGGATGGGGAAAGCAT
AATTGTGTACACAGAGAGGATGTGATTGTAACCTGCTCAGGTGATGCAA
CATGGGGCCTGAGGCTGGTGGGCGGCAGCAACCGCTGCTCGGGAAGACT
GGAGGTGTACTTTCAAGGACGGTGGGGCACAGTGTGTGATGACGGCTGG
AACAGTAAAGCTGCAGCTGTGGTGTGTAGCCAGCTGGACTGCCCATCTT
CTATCATTGGCATGGGTCTGGGAAACGCTTCTACAGGATATGGAAAAAT
TTGGCTCGATGATGTTTCCTGTGATGGAGATGAGTCAGATCTCTGGTCA
TGCAGGAACAGTGGGTGGGGAAATAATGACTGCAGTCACAGTGAAGATG
TTGGAGTGATCTGTTCTGATGCATCGGATATGGAGCTGAGGCTTGTGGG
TGGAAGCAGCAGGTGTGCTGGAAAAGTTGAGGTGAATGTCCAGGGTGCC
GTGGGAATTCTGTGTGCTAATGGCTGGGGAATGAACATTGCTGAAGTTG
TTTGCAGGCAACTTGAATGTGGGTCTGCAATCAGGGTCTCCAGAGAGCC
TCATTTCACAGAAAGAACATTACACATCTTAATGTCGAATTCTGGCTGC
ACTGGAGGGGAAGCCTCTCTCTGGGATTGTATACGATGGGAGTGGAAAC
AGACTGCGTGTCATTTAAATATGGAAGCAAGTTTGATCTGCTCAGCCCA
CAGGCAGCCCAGGCTGGTTGGAGCTGATATGCCCTGCTCTGGACGTGTT
GAAGTGAAACATGCAGACACATGGCGCTCTGTCTGTGATTCTGATTTCT
CTCTTCATGCTGCCAATGTGCTGTGCAGAGAATTAAACTGTGGAGATGC
CATATCTCTTTCTGTGGGAGATCACTTTGGAAAAGGGAATGGTCTAACT
TGGGCCGAAAAGTTCCAGTGTGAAGGGAGTGAAACTCACCTTGCATTAT
GCCCCATTGTTCAACATCCGGAAGACACTTGTATCCACAGCAGAGAAGT
TGGAGTTGTCTGTTCCCGATATACAGATGTCCGACTTGTGAATGGCAAA
TCCCAGTGTGACGGGCAAGTGGAGATCAACGTGCTTGGACACTGGGGCT
CACTGTGTGACACCCACTGGGACCCAGAAGATGCCCGTGTTCTATGCAG
ACAGCTCAGCTGTGGGACTGCTCTCTCAACCACAGGAGGAAAATATATT
GGAGAAAGAAGTGTTCGTGTGTGGGGACACAGGTTTCATTGCTTAGGGA
ATGAGTCACTTCTGGATAACTGTCAAATGACAGTTCTTGGAGCACCTCC
CTGTATCCATGGAAATACTGTCTCTGTGATCTGCACAGGAAGCCTGACC
CAGCCACTGTTTCCATGCCTCGCAAATGTATCTGACCCATATTTGTCTG
CAGTTCCAGAGGGCAGTGCTTTGATCTGCTTAGAGGACAAACGGCTCCG
CCTAGTGGATGGGGACAGCCGCTGTGCCGGGAGAGTAGAGATCTATCAC
GACGGCTTCTGGGGCACCATCTGTGATGACGGCTGGGACCTGAGCGATG
CCCACGTGGTGTGTCAAAAGCTGGGCTGTGGAGTGGCCTTCAATGCCAC
GGTCTCTGCTCACTTTGGGGAGGGGTCAGGGCCCATCTGGCTGGATGAC
CTGAACTGCACAGGAATGGAGTCCCACTTGTGGCAGTGCCCTTCCCGCG
GCTGGGGGCAGCACGACTGCAGGCACAAGGAGGACGCAGGGGTCATCTG
CTCAGAATTCACAGCCTTGAGGCTCTACAGTGAAACTGAAACAGAGAGC
TGTGCTGGGAGATTGGAAGTCTTCTATAACGGGACCTGGGGCAGCGTCG
GCAGGAGGAACATCACCACAGCCATAGCAGGCATTGTGTGCAGGCAGCT
GGGCTGTGGGGAGAATGGAGTTGTCAGCCTCGCCCCTTTATCTAAGACA
GGCTCTGGTTTCATGTGGGTGGATGACATTCAGTGTCCTAAAACGCATA
TCTCCATATGGCAGTGCCTGTCTGCCCCATGGGAGCGAAGAATCTCCAG
CCCAGCAGAAGAGACCTGGATCACATGTGAAGATAGAATAAGAGTGCGT
GGAGGAGACACCGAGTGCTCTGGGAGAGTGGAGATCTGGCACGCAGGCT
CCTGGGGCACAGTGTGTGATGACTCCTGGGACCTGGCCGAGGCGGAAGT
GGTGTGTCAGCAGCTGGGCTGTGGCTCTGCTCTGGCTGCCCTGAGGGAC
GCTTCGTTTGGCCAGGGAACTGGAACCATCTGGTTGGATGACATGCGGT
GCAAAGGAAATGAGTCATTTCTATGGGACTGTCACGCCAAACCCTGGGG
ACAGAGTGACTGTGGACACAAGGAAGATGCTGGCGTGAGGTGCTCTGGA
CAGTCGCTGAAATCACTGAATGCCTCCTCAGGTCATTTAGCACTTATTT
TATCCAGTATCTTTGGGCTCCTTCTCCTGGTTCTGTTTATTCTATTTCT
CACGTGGTGCCGAGTTCAGAAACAAAAACATCTGCCCCTCAGAGTTTCA
ACCAGAAGGAGGGGTTCTCTCGAGGAGAATTTATTCCATGAGATGGAGA
CCTGCCTCAAGAGAGAGGACCCACATGGGACAAGAACCTCAGATGACAC
CCCCAACCATGGTTGTGAAGATGCTAGCGACACATCGCTGTTGGGAGTT
CTTCCTGCCTCTGAAGCCACAAAATGACTTTAGACTTCCAGGGCTCACC
AGATCAACCTCTAAATATCTTTGAAGGAGACAACAACTTTTAAATGAAT
AAAGAGGAAGTCAAGTTGCCCTATGGAAAACTTGTCCAAATAACATTTC
TTGAACAATAGGAGAACAGCTAAATTGATAAAGACTGGTGATAATAAAA
ATTGAATTATGTATATCACTGTTAAAAAAAAAAAAAAAAAA

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “biologic sample” is meant any tissue, cell, fluid, or other material derived from an organism.

By “characteristic DNA copy number variation” is meant that the number of DNA copies on a chromosome varies (i.e., is increased or decreased) relative to the number of DNA copies present in a healthy control cell or organism.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “disease” is meant any condition or disorder that damages or interferes with the normal function of a cell, tissue, or organ. Examples of diseases include thyroid lesions (e.g., benign follicular adenomas (FAs), papillary thyroid carcinomas (PTC) and follicular variant papillary thyroid carcinomas (FVPTCs)).

The invention provides a number of targets that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “invasive disease” is meant a neoplasia or carcinoma that has metastasized or that has a propensity to metastasize.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any analyte (e.g., polypeptide, polynucleotide) or other clinical parameter that is differentially present in a subject having a condition or disease as compared to a control subject (e.g., a person with a negative diagnosis or normal or healthy subject). For example, characteristic DNA copy number variation on any one or more of chromosomes 7, 12, or 22, or an alteration in the expression level of a NDUFA12, NR2C1, FGD6, VEZT and/or GDF3 polypeptide or polynucleotide. In another embodiment, an amplification or deletion of a portion of a chromosome is a marker of the invention.

By “molecularly characterize” is meant detect using assays or tools of molecule biology. Such methods do not include chromosomal karyotyping or cytological methods.

By “mutation” is meant an alteration in the sequence of a polynucleotide or polypeptide relative to a reference sequence. A reference sequence is typically the wild-type sequence.

As used herein, “obtaining” as in “obtaining an agent” includes synthesizing, purchasing, or otherwise acquiring the agent.

By “periodic” is meant at regular intervals. Periodic patient monitoring includes, for example, a schedule of tests that are administered daily, bi-weekly, bi-monthly, monthly, bi-annually, or annually.

By “premalignant state” is meant the state of a cell prior to malignancy.

By “malignant potential” is meant a propensity to become malignant.

By “benign potential” is meant a propensity to remain benign.

By “severity of neoplasia” is meant the degree of pathology. The severity of a neoplasia increases, for example, as the stage or grade of the neoplasia increases.

By “Marker profile” is meant a characterization of the expression or expression level of two or more polypeptides or polynucleotides.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

By “reduces” is meant a negative alteration of at least 10%, 25%, 50%, 75%, or 100%.

By “reference” is meant a standard of comparison. For example, the characteristic DNA copy number or level of NDUFA12, NR2C1, FGD6, VEZT and GDF3 polypeptide or polynucleotide level present in a patient sample may be compared to the level of said polypeptide or polynucleotide present in a corresponding healthy cell or tissue or in a neoplastic cell or tissue that lacks a propensity to metastasize.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100.mu.g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art. For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

By “thyroid lesion” is meant any abnormality present in the thyroid of a subject. Such abnormalities include indeterminate thyroid lesions, as well as benign follicular adenomas (FAs), papillary thyroid carcinomas (PTC) and follicular variant papillary thyroid carcinomas (FVPTCs).

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 is a heatmap depicting an unsupervised hierarchical clustering of 39 thyroid tumors. Only the 10% of segments with the greatest sample-to-sample variation in copy number, as measured by Illumina 550K SNP array, are shown. The tumor samples have been formally clustered on the x-axis in this analysis, while copy number is presented in genomic order on the y-axis. Individual tumors are shown as columns, with tumor subtypes shown in the colored annotation band along the top: follicular adenoma (FA, n=14) in blue, papillary thyroid carcinoma (PTC, n=12) in deep pink, and follicular variant of PTC (FVPTC, n=13) in orange. Each row of the heatmap summarizes copy number in one 25 kb region of the genome, and in all, 11,426 such regions are represented here, selected for highly variable copy number and sorted in chromosome order. In the body of the heatmap, copy number is color coded from bright green (homozygous deletion) to bright red (high amplitude amplifications), as shown in the figure legend.

FIG. 2 shows three panels depicting a graph (top), a plot (middle), and a graph (bottom) that together provide an overview of statistically significant copy number changes. The horizontal axis is the same for all 3 panels, showing genomic location, with chromosomal boundaries depicted as vertical lines. In the middle panel, where the vertical axis shows the 39 tumor samples grouped by subtype, all of the CNVs we identified as statistically significant by permutation test are represented, deletions in green, and amplifications in red. The remaining panels offer a view of the same data, summarized by tumor subtype, depicting the proportion of samples within each subtype having amplifications (top panel) or deletions (bottom panel) on each chromosome.

FIGS. 3A-3E show three chromosome profile graphs, a dot plot, and a log plot, respectively. Mean copy number fold changes on chromosomes 7, 12 and 22 in thyroid tumor subtypes. Calculations were performed after summarizing copy number by gene for each sample. FIGS. 3A-3C shown mean relative copy number on chromosomes 7, 12 and 22, respectively. FAs are shown in blue, FVPTCs in orange and PTCs in pink. In each case, the x-axis gives the physical position of each gene on the chromosome; with log fold copy number shown on the y-axis. Chromosomes 7 and 12 show widespread amplifications in many FAs, chromosome 22 deletions in subsets of the FVPTC and FA samples. A value of 0 corresponds to a ratio of tumor copy number to normal tissue copy number of 1. FIG. 3D shows the log fold copy number for each sample on chromosome 12, calculated by averaging 10 genes selected by ANOVA to distinguish FAs from PTCs and FVPTCs. The horizontal line at log fold=0.07 optimally demarcates benign and malignant tumors. FIG. 3E shows the results of a cross-validated evaluation of this chromosome 12 gene panel by ROC, achieving an AUC of 0.88.

FIGS. 4A-4C show three box plots showing SNP array, expression array, and RT-PCR, respectively, validation of chromosome 12 copy number changes. Five genes selected for validation, NDUFA12, NR2C1, FGD6, VEZT, and GDF3, were averaged to obtain a single, composite value for each sample. Bracket's identify statistically significant between group differences using Welch's t-test; * indicates P<0.05, and ** indicates P<0.01. FIG. 4A shows the average relative copy number of the five selected genes for all samples of each tumor subtype, as measured on the SNP arrays. FIG. 4B shows expression of the 5 genes as measured by cDNA array. The log intensities from expression arrays normalized by matching normal thyroid tissue were averaged across genes to obtain a single estimated value for each sample. (C) Panel C shows copy number estimates as measured by quantitative real-time PCR of genomic DNA. Estimated copy number changes from 15 primer pairs (3 primer pairs for each of the 5 genes) were averaged to obtain a single estimate of chromosome 12 relative copy number for each sample. In total, 100 thyroid tumor-normal paired samples were assayed, including the discovery set of 39 cases and additional samples from a test set of 7 FCs, 5 HCs, 10 FVPTCs, 9 PTCs, 18 FAs, and 12 ANs. For reference, the observed copy number changes for a chromosome 21 region in 3 Down Syndrome patients is shown as an example of a trisomy, while an X chromosome region is measured in 9 normal males compared with 3 normal females as a surrogate for a monosomy.

FIG. 5 is a box plot showing the results of a Real-time PCR assay of Ch12 amplification signature in thyroid tissue and matched FNA samples. Box plots show fold copy number changes (Fold CN, relative to matching normal thyroid tissue) of Ch12 genes in 10 FAs for which both tissue and FNA samples were available. The left panel shows 8 cases (AMP) had shown Fold CN values consistent with amplification in tissue-derived DNA, while 2 cases (WT) showed no amplification. The right panel shows the result of the same real-time PCR assay in matched FNA samples after enrichment for epithelial cells. The normalized Ct value (-delta Ct(Target-Alu)) represents copy number changes for FNA samples normalized for Alu elements, since no matching normal cell sample was available. For reference, results of the same assay on three white blood cell (WBC) samples from patients with benign thyroid disease (multi-nodular hyperplasia) are shown.

FIGS. 6A-6D show a plot, and three smoothed scatter plots illustrating the identification of copy number variation by 550K SNP array analysis. FIG. 6A is a plot showing selection of statistically significant CNVs across the human genome in all 39 thyroid tumor-normal paired tissue samples. The x-axis represents the estimated value of log2 fold copy number variation for each segment identified by CBS method, with 0 representing an equal signal in tumor and matched normal sample. The y-axis indicates the length of each segment of CNV, represented by natural logarithm of SNP count spanning that region. The yellow line indicates the cutoff for identifying copy number amplifications and deletions with statistical significance, which was generated by permutation test with less than 10% type 1 error. The red dots represented copy number amplifications; the green dots represented the copy number deletions. Specifically, segments with log fold change between 0.25 (corresponding to a DNA segment copy number of 2.4) and 1.5 (5.7 copies), and spanning more than 3 SNP sites, as well as segments with log fold change exceeding 1.5 (5.7 copies) and spanning more than 2 SNP sites, were defined as copy number amplifications, while segments with log fold changes between −0.25 (1.7 copies) and −1.75 (0.6 copies), and spanning over 3 SNP sites, as well as those with log fold copy changes less than −1.75 (0.6 copies), and spanning more than 2 SNP sites, were defined as copy number deletions. FIG. 6B depicts an example of several focal events (with length less than 1M bp) of copy number amplification and deletions on chromosome 2, in sample FA_{—}020. The x-axis indicates the position of each SNP marker along chromosome 2; y-axis represents the log2 fold copy number variation for each SNP probe. The smoothed scatter-plot described the regional densities in blue color accounting for the amount of SNPs within the local area. The segments, composed of SNPs with constant copy number changes identified by CBS algorithm, were represented by black solid line; the red arrows highlight the segments as amplifications with statistical significance; the green arrows labeled the segments as deletions with statistical significance. FIG. 6C shows that case FA_{—}785 exhibited a focal high amplification event and large lower amplitude event of chromosomal amplification, labeled by red arrows, on chromosome 17q. FIG. 6D shows that case FVPTC_{—}101 harbored a subtotal 22q deletion, indicated by a green arrow, when compared with paired normal thyroid DNA as control. There are no SNPs on 22p of this acrocentric chromosome.

FIG. 7 illustrates a map of genomic regions of copy number variation selected for the heat map shown in FIG. 1 on a chromosome by chromosome basis. The variation in copy number across all samples is represented as the standard deviation of the log R (signal intensity) ratio, plotted along the pictogram of each chromosome. In order to select the most variable 10% of regions across the genome, a threshold standard deviation of at least 0.09 was necessary. This threshold is represented as a horizontal line in each panel. Only those regions of the genome with the 10% greatest variation in copy number are represented in the heat map shown in FIG. 1. The proportion of chromosome segments reaching this threshold for inclusion in FIG. 1 is indicated as % at the top of each panel.

### DETAILED DESCRIPTION OF THE INVENTION

In general, the invention provides compositions and methods for characterizing thyroid lesions (e.g., benign follicular adenomas (FAs), papillary thyroid carcinomas (PTC) and follicular variant papillary thyroid carcinomas (FVPTCs)).

The invention is based, at least in part, on the discovery that thyroid tumor subtypes show characteristic DNA copy number variation (CNV) patterns when analysed using high-resolution single nucleotide polymorphism (SNP) arrays for the genomic characterizations of thyroid tumors. In order to maximize the statistical power of the initial analysis, the three tumor subtypes most commonly leading to an ambiguous pre-operative diagnosis: papillary thyroid carcinomas (PTC), follicular variant papillary thyroid carcinomas (FVPTCs), and follicular adenomas (Fas) were selected for characterization. Follicular carcinomas (FCs) are much less common, and were therefore not included in our initial genome-wide screen.

### Diagnosis of Thyroid Cancer

Fine needle aspiration is the best diagnostic tool for pre-operative evaluation of thyroid nodules, but is often inconclusive as guide for surgical management. As detailed below, thyroid tumor subtypes show characteristic DNA copy number variation (CNV) patterns. The present invention provides for the characterization of such profiles, thereby improving preoperative classification. The study cohorts included benign follicular adenomas (FA), classic papillary thyroid carcinomas (PTC) and follicular variant papillary thyroid carcinomas (FVPTC), the three subtypes most commonly associated with inconclusive preoperative cytopathology.

Tissue and FNA samples were obtained from subjects that underwent partial or complete thyroidectomy for malignant or indeterminate thyroid lesions. Pairs of tumor tissue and matching normal thyroid tissue derived DNA were compared using 550K SNP arrays and significant differences in characteristic DNA copy number variation patterns were identified between tumor subtypes.

Segmental amplifications in chromosomes 7 and 12 were more common in follicular adenomas than in papillary thyroid carcinomas or follicular variant papillary thyroid carcinomas. Additionally, a subset of follicular adenomas and follicular variant papillary thyroid carcinomas showed deletions in Ch22. The present study also identified five CNV-associated genes capable of discriminating between follicular adenomas and papillary thyroid carcinomas/follicular variant papillary thyroid carcinomas. These genes correctly classified 90% of cases. These five chromosome 12 genes were validated by quantitative genomic PCR and gene expression array analyses on the same patient cohort. The five-gene signature was then successfully validated against an independent test cohort of benign and malignant tumor samples. Finally, a feasibility study was performed on matched FA-derived intraoperative FNA samples. This study correctly distinguished follicular adenomas harboring the chromosome 12 amplification signature from follicular adenomas without the chromosome 12 amplification. Thus, thyroid tumor subtypes possess characteristic genomic profiles. These profiles provide for the identification of structural genetic changes in thyroid tumor subtypes.

### Diagnostic Assays

The present invention provides a number of diagnostic assays that are useful for the identification or characterization of a thyroid lesion. In one embodiment, a thyroid tumor subtype possesses a characteristic genomic profile that identifies it as a benign follicular adenoma (FA), classic papillary thyroid carcinoma (PTC) or follicular variant papillary thyroid carcinoma. To separate the thyroid lesions into subtypes characteristic DNA copy number variation patterns are identified. Such patterns include characteristic DNA copy number variation at one or more of chromosomes 7, 12 and 22. Characterizing the thyroid tumor by subtype is useful for preoperative classification.

In certain embodiments, alterations in chromosomes 7, 12, and 22 are assayed in combination with telomerase activity or expression levels. Human telomerase is a specialized ribonucleoprotein composed of two components, a reverse transcriptase protein subunit (hTERT) (J. Feng, Science 269, 1236-1241 (1995); T. M. Nakamura, Science 277, 911-912 (1997)), as well as several associated proteins. Telomerase directs the synthesis of telomeric repeats at chromosome ends, using a short sequence within the RNA component as a template. Telomerase is considered to be an almost universal marker for human cancer, its effect on telomere length playing a crucial role in evading replicative senescence. Telomerase refers to the ribonucleoprotein complex that reverse transcribes a portion of its RNA subunit during the synthesis of G-rich DNA at the 3′ end of each chromosome in most eukaryotes, thus compensating for the inability of the normal DNA replication machinery to fully replicate chromosome termini. The human telomerase holoenzyme minimally comprises two essential components, a reverse transcriptase protein subunit (hTERT), and the “RNA component of human telomerase.” The RNA component of telomerase from diverse species differ greatly in their size and share little sequence homology, but do appear to share common secondary structures, and important common features include a template, a 5′ template boundary element, a large loop including the template and putative pseudoknot, referred to herein as the “pseudoknot/template region,” and a loop-closing helix. Human telomerase activity is described for example by V. M. Tesmer Mol Cell Biol. 19(9):6207-160 (1999) and US Patent Application No. 20110257251, which is incorporated herein by reference in its entirety for all purposes.

In other embodiments, characteristic DNA copy number variation is used in combination with HRas (Omim No. 190020; Cytogenetic location: 11p15.5, Genomic coordinates (GRCh37): 11:532,241-535,549) or Nras (Omim No. 164790; Cytogenetic location: 1p13.2 Genomic coordinates (GRCh37): 1:115,247,084-115,259,514).

While the examples provided below describe methods of detecting characteristic DNA copy number variation using SNP array analysis, quantitative Real-time genomic PCR analysis, gene expression array analysis, or transcriptome array analysis, the skilled artisan appreciates that the invention is not limited to such methods. Characteristic DNA copy number variation levels are quantifiable by any standard method, such methods include, but are not limited to real-time PCR, bisulfite genomic DNA sequencing, restriction enzyme-PCR, DNA microarray analysis based on fluorescence or isotope labeling, and mass spectroscopy.

In one embodiment, a desired genomic target (e.g., portions of chromosomes 7, 12 and/or 22) is analysed.

Characteristic DNA copy number variation or gene set copy number or expression can be measured using the polymerase chain reaction (PCR). The amplified product is then detected using standard methods known in the art. In one embodiment, a PCR product (i.e., amplicon) or real-time PCR product is detected by probe binding. In one embodiment, probe binding generates a fluorescent signal, for example, by coupling a fluorogenic dye molecule and a quencher moiety to the same or different oligonucleotide substrates (e.g., TaqMan® (Applied Biosystems, Foster City, Calif., USA), Molecular Beacons (see, for example, Tyagi et al., Nature Biotechnology 14(3):303-8, 1996), Scorpions® (Molecular Probes Inc., Eugene, Oreg., USA)). In another example, a PCR product is detected by the binding of a fluorogenic dye that emits a fluorescent signal upon binding (e.g., SYBR® Green (Molecular Probes)).

The characteristic DNA copy number variation defines the profile of a thyroid carcinoma. The DNA copy number present in a biological sample is compared to a reference. In one embodiment, the reference is the DNA copy number present in a control sample obtained from a patient that does not have a carcinoma. In yet another embodiment, the reference is a reference level or a standardized curve.

Methods for measuring DNA copy number as described herein is used, alone or in combination with other methods, to characterize the thyroid carcinoma. In one embodiment the carcinoma is characterized to determine its stage or grade. Grading is used to describe how abnormal or aggressive the neoplastic cells appear, while staging is used to describe the extent of the neoplasia.

The present invention features diagnostic assays for the characterization of thyroid lesions (e.g., benign follicular adenomas, papillary thyroid carcinomas, and follicular variant papillary thyroid carcinomas). In addition to detecting DNA copy number changes, polypeptide and polynucleotide markers may also be used as diagnostics. In one embodiment, levels of any one or more of the following markers: NDUFA12, NR2C1, FGD6, VEZT and GDF3 are measured in a subject sample and used to characterize a thyroid lesion. In other embodiments, levels of any one or more of NDUFA12, NR2C1, FGD6, VEZT and GDF3 are characterized in a subject sample. Standard methods may be used to measure levels of a marker in any biological sample. Biological samples include tissue samples (e.g., cell samples, fine needle aspiration, biopsy samples). Methods for measuring levels of polypeptide include immunoassay, ELISA, western blotting and radioimmunoassay. Elevated levels of any of NDUFA12, NR2C1, FGD6, VEZT and GDF3 alone or in combination with one or more additional markers are used to characterize a thyroid lesion. The increase in NDUFA12, NR2C1, FGD6, VEZT and GDF3 levels may be by at least about 10%, 25%, 50%, 75% or more. In one embodiment, any increase in a marker of the invention can be used to characterize a thyroid lesion.

Any suitable method can be used to detect one or more of the markers described herein. Successful practice of the invention can be achieved with one or a combination of methods that can detect and, preferably, quantify the markers. These methods include, without limitation, hybridization-based methods, including those employed in biochip arrays, mass spectrometry (e.g., laser desorption/ionization mass spectrometry), fluorescence (e.g. sandwich immunoassay), surface plasmon resonance, ellipsometry and atomic force microscopy. Expression levels of markers (e.g., polynucleotides or polypeptides) are compared by procedures well known in the art, such as RT-PCR, Northern blotting, Western blotting, flow cytometry, immunocytochemistry, binding to magnetic and/or antibody-coated beads, in situ hybridization, fluorescence in situ hybridization (FISH), flow chamber adhesion assay, ELISA, microarray analysis, or colorimetric assays. Methods may further include, one or more of electrospray ionization mass spectrometry (ESI-MS), ESI-MS/MS, ESI-MS/(MS)ⁿ, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), atmospheric pressure chemical ionization mass spectrometry (APCI-MS), APCI-MS/MS, APCI-(MS)ⁿ, atmospheric pressure photoionization mass spectrometry (APPI-MS), APPI-MS/MS, and APPI-(MS)ₙ, quadrupole mass spectrometry, fourier transform mass spectrometry (FTMS), and ion trap mass spectrometry, where n is an integer greater than zero.

Detection methods may include use of a biochip array. Biochip arrays useful in the invention include protein and polynucleotide arrays. One or more markers are captured on the biochip array and subjected to analysis to detect the level of the markers in a sample.

Markers may be captured with capture reagents immobilized to a solid support, such as a biochip, a multiwell microtiter plate, a resin, or a nitrocellulose membrane that is subsequently probed for the presence or level of a marker. Capture can be on a chromatographic surface or a biospecific surface. For example, a sample containing the markers may be used to contact the active surface of a biochip for a sufficient time to allow binding. Unbound molecules are washed from the surface using a suitable eluant, such as phosphate buffered saline. In general, the more stringent the eluant, the more tightly the proteins must be bound to be retained after the wash.

Upon capture on a biochip, analytes can be detected by a variety of detection methods selected from, for example, a gas phase ion spectrometry method, an optical method, an electrochemical method, atomic force microscopy and a radio frequency method. In one embodiment, mass spectrometry, and in particular, SELDI, is used. Optical methods include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Immunoassays in various formats (e.g., ELISA) are popular methods for detection of analytes captured on a solid phase. Electrochemical methods include voltametry and amperometry methods. Radio frequency methods include multipolar resonance spectroscopy.

Mass spectrometry (MS) is a well-known tool for analyzing chemical compounds. Thus, in one embodiment, the methods of the present invention comprise performing quantitative MS to measure the serum peptide marker. The method may be performed in an automated (Villanueva, et al., Nature Protocols (2006) 1(2):880-891) or semi-automated format. This can be accomplished, for example with MS operably linked to a liquid chromatography device (LC-MS/MS or LC-MS) or gas chromatography device (GC-MS or GC-MS/MS). Methods for performing MS are known in the field and have been disclosed, for example, in US Patent Application Publication Nos: 20050023454; 20050035286; U.S. Pat. No. 5,800,979 and references disclosed therein.

In an additional embodiment of the methods of the present invention, multiple markers are measured. The use of multiple markers (e.g., two or more of NDUFA12, NR2C1, FGD6, VEZT and GDF3) increases the predictive value of the test and provides greater utility in diagnosis, toxicology, patient stratification and patient monitoring. The process called “Pattern recognition” detects the patterns formed by multiple markers greatly improves the sensitivity and specificity of clinical proteomics for predictive medicine. Subtle variations in data from clinical samples indicate that certain patterns of protein expression can predict phenotypes such as the presence or absence of a certain disease, a particular stage of cancer-progression, or a positive or adverse response to drug treatments. While particular embodiments have been disclosed with respect to the detection of specific amplification of chromosome 12 and/or 7 by the use of specific markers (e.g., NDUFA12, NR2C1, FGD6, VEZT and GDF3), it is contemplated within the scope of the disclosure that any marker or markers residing within the copy number variation region may be used.

Expression levels of particular nucleic acids or polypeptides are correlated with thyroid carcinoma, and thus are useful in diagnosis. Antibodies that bind a polypeptide described herein, oligonucleotides or longer fragments derived from a nucleic acid sequence described herein (e.g., an NDUFA12, NR2C1, FGD6, VEZT and GDF3 nucleic acid sequence), or any other method known in the art may be used to monitor expression of a polynucleotide or polypeptide of interest. Detection of an alteration relative to a normal, reference sample can be used as a diagnostic indicator of thyroid carcinoma. In particular embodiments, an increase in expression of a NDUFA12, NR2C1, FGD6, VEZT and GDF3 polypeptide is indicative of thyroid carcinoma or the propensity to develop thyroid carcinoma. In other embodiments, a 2, 3, 4, 5, or 6-fold change in the level of a marker of the invention is indicative of thyroid carcinoma. In yet another embodiment, an expression profile that characterizes alterations in the expression two or more markers is correlated with a particular disease state (e.g., thyroid carcinoma). Such correlations are indicative of thyroid carcinoma or the propensity to develop thyroid carcinoma. In one embodiment, a thyroid carcinoma can be monitored using the methods and compositions of the invention.

In one embodiment, the level of one or more markers is measured on at least two different occasions and an alteration in the levels as compared to normal reference levels over time is used as an indicator of thyroid carcinoma or the propensity to develop thyroid carcinoma. The level of marker in a subject having thyroid carcinoma or the propensity to develop such a condition may be altered by as little as 10%, 20%, 30%, or 40%, or by as much as 50%, 60%, 70%, 80%, or 90% or more relative to the level of such marker in a normal control.

The diagnostic methods described herein can be used individually or in combination with any other diagnostic method described herein for a more accurate diagnosis of the presence or severity of thyroid carcinoma.

As indicated above, the invention provides methods for aiding a human cancer diagnosis using one or more markers, as specified herein. These markers can be used alone, in combination with other markers in any set, or with entirely different markers in aiding human cancer diagnosis. The markers are differentially present in samples of a human cancer patient and a normal subject in whom human cancer is undetectable. Therefore, detection of one or more of these markers in a person would provide useful information regarding the probability that the person may have thyroid carcinoma or regarding the aggressiveness of the thyroid carcinoma.

The detection of a marker, a molecular profile, or a characteristic DNA copy number variation is correlated with a probable diagnosis of cancer. The correlation may take into account the amount of the marker or markers in the sample compared to a control amount of the marker or markers (e.g., in normal subjects or in non-cancer subjects such as where cancer is undetectable). A control can be, e.g., the average or median amount of marker present in comparable samples of normal subjects in normal subjects or in non-cancer subjects such as where cancer is undetectable. The control amount is measured under the same or substantially similar experimental conditions as in measuring the test amount. As a result, the control can be employed as a reference standard, where the normal (non-cancer) phenotype is known, and each result can be compared to that standard, rather than re-running a control.

Accordingly, a marker profile may be obtained from a subject sample and compared to a reference marker profile obtained from a reference population, so that it is possible to classify the subject as belonging to or not belonging to the reference population. The correlation may take into account the presence or absence of the markers in a test sample and the frequency of detection of the same markers in a control. The correlation may take into account both of such factors to facilitate determination of cancer status.

In certain embodiments of the methods of qualifying cancer status, the methods further comprise managing subject treatment based on the status. The invention also provides for such methods where the markers (or specific combination of markers) are measured again after subject management. In these cases, the methods are used to monitor the status of the cancer, e.g., response to cancer treatment, remission of the disease or progression of the disease.

The markers of the present invention have a number of other uses. For example, they can be used to monitor responses to certain treatments of human cancer. In yet another example, the markers can be used in heredity studies. For instance, certain markers may be genetically linked. This can be determined by, e.g., analyzing samples from a population of human cancer subjects whose families have a history of cancer. The results can then be compared with data obtained from, e.g., cancer subjects whose families do not have a history of cancer. The markers that are genetically linked may be used as a tool to determine if a subject whose family has a history of cancer is pre-disposed to having cancer.

Any marker, individually, is useful in aiding in the determination of cancer status. First, the selected marker is detected in a subject sample using the methods described herein. Then, the result is compared with a control that distinguishes cancer status from non-cancer status. As is well understood in the art, the techniques can be adjusted to increase sensitivity or specificity of the diagnostic assay depending on the preference of the diagnostician.

While individual markers are useful diagnostic markers, in some instances, a combination of markers provides greater predictive value than single markers alone. The detection of a plurality of markers (or absence thereof, as the case may be) in a sample can increase the percentage of true positive and true negative diagnoses and decrease the percentage of false positive or false negative diagnoses. Thus, preferred methods of the present invention comprise the measurement of more than one marker.

### Microarrays

As reported herein, a number of markers (e.g., a characteristic DNA copy number variation, NDUFA12, NR2C1, FGD6, VEZT and GDF3) have been identified that are associated with various thyroid lesions (e.g., benign follicular adenomas, papillary thyroid carcinomas, and follicular variant papillary thyroid carcinomas). Methods for assaying the characteristic DNA copy number variation or the expression of NDUFA12, NR2C1, FGD6, VEZT and GDF3 gene or polypeptide expression are useful for characterizing thyroid carcinoma. In particular, the invention provides diagnostic methods and compositions useful for identifying a molecular profile that characterizes a thyroid lesion.

The polypeptides and nucleic acid molecules of the invention are useful as hybridizable array elements in a microarray. The array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making polypeptide microarrays are described, for example, by Ge (Nucleic Acids Res. 28: e3. i-e3. vii, 2000), MacBeath et al., (Science 289:1760-1763, 2000), Zhu et al. (Nature Genet. 26:283-289), and in U.S. Pat. No. 6,436,665, hereby incorporated by reference.

### Protein Microarrays

Proteins (e.g., NDUFA12, NR2C1, FGD6, VEZT and GDF3) may be analyzed using protein microarrays. Such arrays are useful in high-throughput low-cost screens to identify alterations in the expression or post-translation modification of a polypeptide of the invention, or a fragment thereof. In particular, such microarrays are useful to identify a protein whose expression is altered in thyroid carcinoma. In one embodiment, a protein microarray of the invention binds a marker present in a subject sample and detects an alteration in the level of the marker. Typically, a protein microarray features a protein, or fragment thereof, bound to a solid support. Suitable solid supports include membranes (e.g., membranes composed of nitrocellulose, paper, or other material), polymer-based films (e.g., polystyrene), beads, or glass slides. For some applications, proteins (e.g., antibodies that bind a marker of the invention) are spotted on a substrate using any convenient method known to the skilled artisan (e.g., by hand or by inkjet printer).

The protein microarray is hybridized with a detectable probe. Such probes can be polypeptide, nucleic acid molecules, antibodies, or small molecules. For some applications, polypeptide and nucleic acid molecule probes are derived from a biological sample taken from a patient, such as a homogenized tissue sample (e.g. a tissue sample obtained by biopsy); or a cell isolated from a patient sample. Probes can also include antibodies, candidate peptides, nucleic acids, or small molecule compounds derived from a peptide, nucleic acid, or chemical library. Hybridization conditions (e.g., temperature, pH, protein concentration, and ionic strength) are optimized to promote specific interactions. Such conditions are known to the skilled artisan and are described, for example, in Harlow, E. and Lane, D., Using Antibodies: A Laboratory Manual. 1998, New York: Cold Spring Harbor Laboratories. After removal of non-specific probes, specifically bound probes are detected, for example, by fluorescence, enzyme activity (e.g., an enzyme-linked calorimetric assay), direct immunoassay, radiometric assay, or any other suitable detectable method known to the skilled artisan.

### Nucleic Acid Microarrays

To produce a nucleic acid microarray, oligonucleotides may be synthesized or bound to the surface of a substrate using a chemical coupling procedure and an ink jet application apparatus, as described in PCT application WO95/251116 (Baldeschweiler et al.), incorporated herein by reference. Alternatively, a gridded array may be used to arrange and link cDNA fragments or oligonucleotides to the surface of a substrate using a vacuum system, thermal, UV, mechanical or chemical bonding procedure.

A nucleic acid molecule (e.g. RNA or DNA) derived from a biological sample may be used to produce a hybridization probe as described herein. The biological samples are generally derived from a patient as a tissue sample (e.g. a tissue sample obtained by biopsy). For some applications, cultured cells or other tissue preparations may be used. The mRNA is isolated according to standard methods, and cDNA is produced and used as a template to make complementary RNA suitable for hybridization. Such methods are known in the art. The RNA is amplified in the presence of fluorescent nucleotides, and the labeled probes are then incubated with the microarray to allow the probe sequence to hybridize to complementary oligonucleotides bound to the microarray.

Incubation conditions are adjusted such that hybridization occurs with precise complementary matches or with various degrees of less complementarity depending on the degree of stringency employed. For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 3° C., more preferably of at least about 37 C., and most preferably of at least about 42 C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30 C in 750 mM NaCl, 75 mM trisodium citrate, and 1% SDS. In a more preferred embodiment, hybridization will occur at 37 C. in 500 mM NaCl, 50 mM trisodium citrate, 1% SDS, 35% formamide, and 100 μg/ml denatured salmon sperm DNA (ssDNA). In a most preferred embodiment, hybridization will occur at 42 C in 250 mM NaCl, 25 mM trisodium citrate, 1% SDS, 50% formamide, and 200 μg/ml ssDNA. Useful variations on these conditions will be readily apparent to those skilled in the art.

The removal of nonhybridized probes may be accomplished, for example, by washing. The washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25 C., more preferably of at least about 42.degree. C., and most preferably of at least about 68 C. In a preferred embodiment, wash steps will occur at 25 C in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a most preferred embodiment, wash steps will occur at 68 C in 15 mM NaCl, 1.5 mM trisodium citrate, and 0.1% SDS. Additional variations on these conditions will be readily apparent to those skilled in the art.

A detection system may be used to measure the absence, presence, and amount of hybridization for all of the distinct nucleic acid sequences simultaneously (e.g., Heller et al., Proc. Natl. Acad. Sci. 94:2150-2155, 1997). Preferably, a scanner is used to determine the levels and patterns of fluorescence.

### Selection of a Treatment Method

After a subject is diagnosed as having a thyroid lesion, the lesion is characterized to determine its subtype and or its benign or malignant potential. If the thyroid lesion is benign and is unlikely to have malignant potential, no treatment may be necessary. However, the lesion may be monitored periodically (annually, biannually) to confirm that no malignancy is presence. If the thyroid lesion has malignant potential a method of treatment (e.g., surgery) is selected. Such treatment may be combined with any one or a number of standard treatment regimens.

### Patient Monitoring

The diagnostic methods of the invention are also useful for monitoring the course of a thyroid cancer in a patient or for assessing the efficacy of a therapeutic regimen. In one embodiment, the diagnostic methods of the invention are used periodically to monitor the characteristic DNA copy number variation or the copy number or expression of a gene set (e.g., NDUFA12, NR2C1, FGD6, VEZT and GDF3). In one example, the thyroid carcinoma is characterized using a diagnostic assay of the invention prior to administering therapy. This assay provides a baseline that describes the DNA copy number prior to treatment. Additional diagnostic assays are administered during the course of therapy to monitor the efficacy of a selected therapeutic regimen.

### Kits

The invention also provides kits for the diagnosis or monitoring of a thyroid carcinoma in a biological sample obtained from a subject. In various embodiments, the kit includes materials for SNP array analysis, quantitative Real-time genomic PCR analysis, gene expression array analysis, or transcriptome array analysis. In yet other embodiments, the kit comprises a sterile container which contains the primer or probe; such containers can be boxes, ampules, bottles, vials, tubes, bags, pouches, blister-packs, or other suitable container form known in the art. Such containers can be made of plastic, glass, laminated paper, metal foil, or other materials suitable for holding nucleic acids. The instructions will generally include information about the use of the primers or probes described herein and their use in diagnosing a thyroid carcinoma. Preferably, the kit further comprises any one or more of the reagents described in the diagnostic assays described herein. In other embodiments, the instructions include at least one of the following: description of the primer or probe; methods for using the enclosed materials for the diagnosis of a neoplasia; precautions; warnings; indications; clinical or research studies; and/or references. The instructions may be printed directly on the container (when present), or as a label applied to the container, or as a separate sheet, pamphlet, card, or folder supplied in or with the container.

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

It should be appreciated that the invention should not be construed to be limited to the examples that are now described; rather, the invention should be construed to include any and all applications provided herein and all equivalent variations within the skill of the ordinary artisan.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Characteristic Genomic Copy Number Variation Patterns are Associated with FAs, FVPTCs, and PTCs

Using Illumina 550K SNP arrays, genome-wide DNA copy number changes were investigated in 39 thyroid tumors (14 FAs, 13 FVPTCs, and 12 PTCs) with paired normal thyroid tissue samples from the same patients as controls (See Table 1 and Table 2 for clinical patient information).

TABLE 1
Clinical information summary of tissue
sample cases used in this study

| | | | | |
|---|---|---|---|---|
| Tumor | Total | Median | Median | Tumor |
| Type | (M/F) | Age | Size (cm) | Stage (n) |

Discovery patient cohort for SNP array analysis

| | | | | |
|---|---|---|---|---|
| FA | 3/11 | 42 | 3.2 | |
| FVPTC | 2/11 | 47 | 4 | I (8), II (2), III (2), IV (1) |
| PTC | 3/9 | 42.5 | 2.5 | I (7), II (1), III (1), IV (3) |

Validation patient cohort

| | | | | |
|---|---|---|---|---|
| FA | 6/12 | 51 | 2.7 | |
| FVPTC | 2/8 | 37 | 3.2 | I (6), II (2), III (1), IV (1) |
| PTC | 3/6 | 48 | 2 | I (6), II (1), III (1), IV (1) |
| FC | 5/2 | 55 | 4 | I (4), III (3) |
| HC | 2/3 | 56 | 3.5 | I (1), II (1), III (2), IV (1) |
| AN | 2/10 | 50.5 | 2.9 | |
| Total | 23/61 | 46 | 3.2 | |

TABLE 2
Clinical Information of the thyroid tumor samples used in this study.

| | | | | | | | |
|---|---|---|---|---|---|---|---|
| Subtype_Case no. | | Tumor | | | Invasive | Genetic | BRAF |
| (Id) | Age/Sex | size (cm) | TNM | Stage | status | Cluster* | mutation |

Initial set for SNP array analysis

| | | | | | | | |
|---|---|---|---|---|---|---|---|
| FA_020 | 45/F | 10 | | | | Cluster1 | |
| FA_221 | 45/F | 2 | | | | Cluster1 | |
| FA_588 | 39/M | 3.3 | | | | Cluster1 | |
| FA_605 | 71/M | 4 | | | | Cluster1 | |
| FA_760 | 53/F | 2.5 | | | | Cluster1 | |
| FA_653 | 50/F | 3 | | | | Cluster1 | |
| FA_779 | 34/M | 1.5 | | | | Cluster1 | |
| FA_394 | 51/F | 3.5 | | | | Cluster2 | |
| FA_413 | 51/F | 1.2 | | | | Cluster2 | |
| FA_722 | 34/F | 5 | | | | Cluster2 | |
| FA_785 | 30/F | 3 | | | | Cluster2 | |
| FA_410 | 32/F | 3.8 | | | | Cluster3 | |
| FA_419 | 24/F | 1.5 | | | | Cluster3 | |
| FA_803 | 25/F | 5 | | | | Cluster3 | |
| FVPTC_137 | 18/M | 5 | T3N0M0 | I | encapsulated | Cluster2 | Negative |
| FVPTC_189 | 68/F | 4.2 | T3N0M0 | III | encapsulated | Cluster2 | Negative |
| FVPTC_210 | 48/F | 1.3 | T2N0M0 | II | encapsulated | Cluster2 | Positive |
| FVPTC_236 | 47/F | 4.5 | T3N0M0 | III | invasive | Cluster2 | Negative |
| FVPTC_297 | 55/F | 4 | T2NXM0 | II | invasive | Cluster2 | Negative |
| FVPTC_301 | 20/F | 5 | T3N0M0 | I | encapsulated | Cluster2 | Negative |
| FVPTC_631 | 58/F | 1.4 | T1NXM0 | I | invasive | Cluster2 | Positive |
| FVPTC_741 | 62/F | 1.5 | T1NXM0 | I | invasive | Cluster2 | Negative |
| FVPTC_322 | 32/F | 1.2 | T1NXM0 | I | invasive | Cluster2 | Negative |
| FVPTC_739 | 60/M | 6 | T3N1bM0 | IV | invasive | Cluster2 | Negative |
| FVPTC_101 | 40/F | 1.7 | T1NXM0 | I | invasive | Cluster3 | Negative |
| FVPTC_358 | 43/F | 5 | T3NXM0 | I | invasive | Cluster3 | Negative |
| FVPTC_374 | 30/F | 4 | T3NXM0 | I | invasive | Cluster3 | Negative |
| PTC_501 | 35/F | 5 | T3NxM0 | I | invasive | Cluster1 | Negative |
| PTC_120 | 44/F | 2 | T1NXM0 | I | encapsulated | Cluster2 | Negative |
| PTC_141 | 51/M | 3.5 | T4N1M0 | IV | invasive | Cluster2 | Positive |
| PTC_199 | 21/F | 3.7 | T3N1M0 | I | invasive | Cluster2 | Negative |
| PTC_251 | 64/M | 4 | T4NXM0 | IV | invasive | Cluster2 | Positive |
| PTC_392 | 41/F | 5.2 | T3N1M1 | II | invasive | Cluster2 | Negative |
| PTC_596 | 62/F | 0.8 | T1N1aM0 | III | invasive | Cluster2 | Negative |
| PTC_717 | 59/F | 0.5 | T1N0M0 | I | invasive | Cluster2 | Negative |
| PTC_726 | 59/F | 2.5 | T4aN0M1 | IV | invasive | Cluster2 | Positive |
| PTC_749 | 27/F | 1 | T1N0M0 | I | invasive | Cluster2 | Positive |
| PTC_791 | 27/M | 2.1 | T2N1aM0 | I | invasive | Cluster2 | Negative |
| PTC_801 | 40/F | 2.4 | T3N1aM0 | I | invasive | Cluster2 | Positive |

Validation Set

| | | | | | | |
|---|---|---|---|---|---|---|
| FA_008 | 62/M | 4.5 | | | | |
| FA_202 | 38/M | 3.7 | | | | |
| FA_584 | 41/F | 1.5 | | | | |
| FA_830 | 60/F | 5.5 | | | | |
| FA_833 | 77/M | 3 | | | | |
| FA_848 | 53/M | 2.7 | | | | |
| FA_889 | 42/F | 3 | | | | |
| FA_892 | 41/F | 8 | | | | |
| FA_921 | 46/F | 1.9 | | | | |
| FA_1002 | 53/F | 3.2 | | | | |
| FA_1017 | 52/F | 2.2 | | | | |
| FA_019 | 53/F | 1.1 | | | | |
| FA_508 | 50/F | 2.6 | | | | |
| FA_579 | 47/M | 2.5 | | | | |
| FA_612 | 36/F | 3.2 | | | | |
| FA_641 | 52/M | 0.8 | | | | |
| FA_707 | 23/F | 1.5 | | | | |
| FA_763 | 52/F | 1.6 | | | | |
| FVPTC_014 | 32/F | 4 | T4NXMX | I | invasive | Negative |
| FVPTC_096 | 37/F | 4.3 | T3NXMX | I | encapsulated | Negative |
| FVPTC_121 | 58/F | 2.8 | T2NXMX | II | encapsulated | Negative |
| FVPTC_124 | 19/M | 2.3 | T2NXMX | I | invasive | Negative |
| FVPTC_844 | 30/F | 2 | T1N0MX | I | invasive | Negative |
| FVPTC_154 | 46/F | 3.2 | T2NXMX | II | invasive | Negative |
| FVPTC_904 | 54/F | 4.8 | T3N1aMX | III | invasive | Negative |
| FVPTC_739 | 60/M | 6 | T3N1bMX | IVa | invasive | Negative |
| FVPTC_834 | 37/F | 2.4 | T2N0MX | I | encapsulated | Negative |
| FVPTC_1203 | 32/F | 3.2 | T2N0MX | I | encapsulated | Negative |
| PTC_143 | 37/F | 1.5 | T4NXMX | I | invasive | Negative |
| PTC_158 | 66/M | 1 | T1MXNX | I | encapsulated | Negative |
| PTC_223 | 69/F | 1.5 | T4NXMX | IV | invasive | Positive |
| PTC_388 | 32/M | 2.5 | T3N1MX | I | invasive | Negative |
| PTC_487 | 40/F | 2 | T3N1aMX | I | invasive | Negative |
| PTC_568 | 52/F | 2.5 | T2N0MX | II | encapsulated | Negative |
| PTC_614 | 57/F | 2 | T1NXMX | I | encapsulated | Positive |
| PTC_639 | 44/M | 2 | T1NXMX | I | invasive | Negative |
| PTC_661 | 48/F | 4 | T3N1aMX | III | invasive | Positive |
| FC_1 | 60/F | 5 | T3NXM0 | III | encapsulated | |
| FC_2 | 55/M | 2 | T1N0M0 | I | encapsulated | |
| FC_3 | 37/M | 2 | T1NXM0 | I | encapsulated | |
| FC_4 | 70/M | 4 | T3NXM0 | III | encapsulated | |
| FC_5 | 27/M | 6.5 | T3NXM0 | I | invasive | |
| FC_6 | 43/F | 2.7 | T2NXM0 | I | invasive | |
| FC_7 | 67/M | 5.5 | T3NXM0 | III | Invasive | |
| HC_1 | 46/M | 3.5 | T3NXM1 | IV | invasive | |
| HC_2 | 41/F | 3 | T2NXM0 | I | encapsulated | |
| HC_3 | 87/F | 6 | T3NXM0 | III | encapsulated | |
| HC_4 | 70/F | 2 | T2N0M0 | II | encapsulated | |
| HC_5 | 56/M | 7 | T3NXM0 | III | invasive | |
| AdN_1017 | 52/F | 2.2 | | | | |
| AdN_1022 | 53/F | 4.5 | | | | |
| AdN_1024 | 31/F | 4 | | | | |
| AdN_1073 | 41/F | 5 | | | | |
| AdN_1088 | 57/F | 0.3 | | | | |
| AdN_1095 | 59/F | 2 | | | | |
| AdN_1099 | 33/F | 4 | | | | |
| AdN_862 | 49/M | 2 | | | | |
| AdN_884 | 27/F | 4.5 | | | | |
| AdN_907 | 59/M | 3 | | | | |
| AdN_946 | 32/F | 2.8 | | | | |
| AdN_644 | 52/F | 2.4 | | | | |

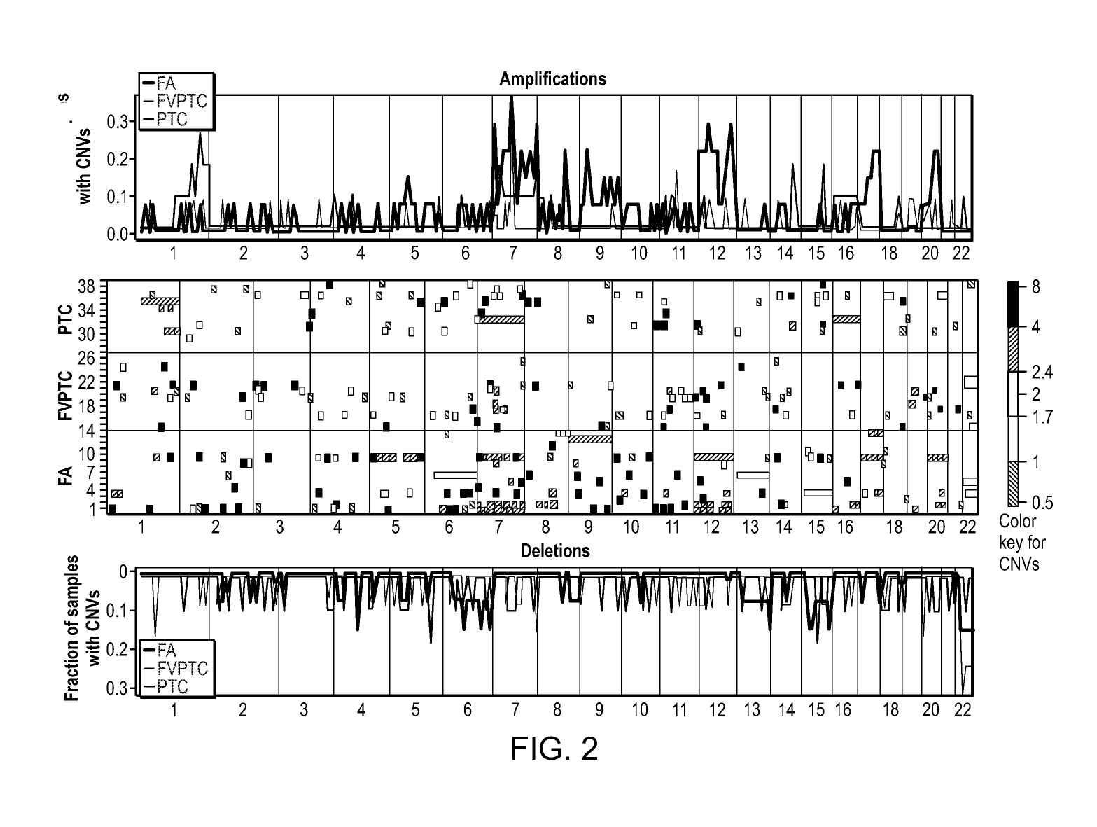

*Cluster 1 is characterized by amplifications of chromosomes 7 and 12; cluster 2 has no significant genomic aberrations; cluster 3 is distinguished by deletion of chromosome 22 (as labeled in FIG. 2).

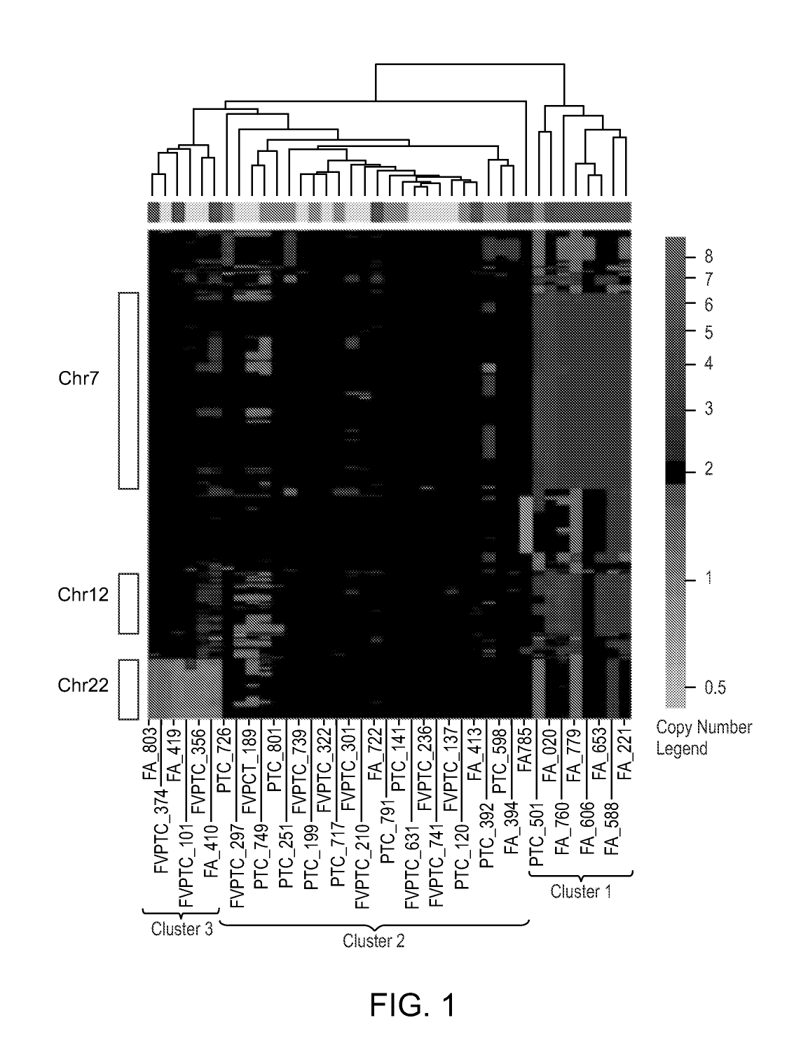

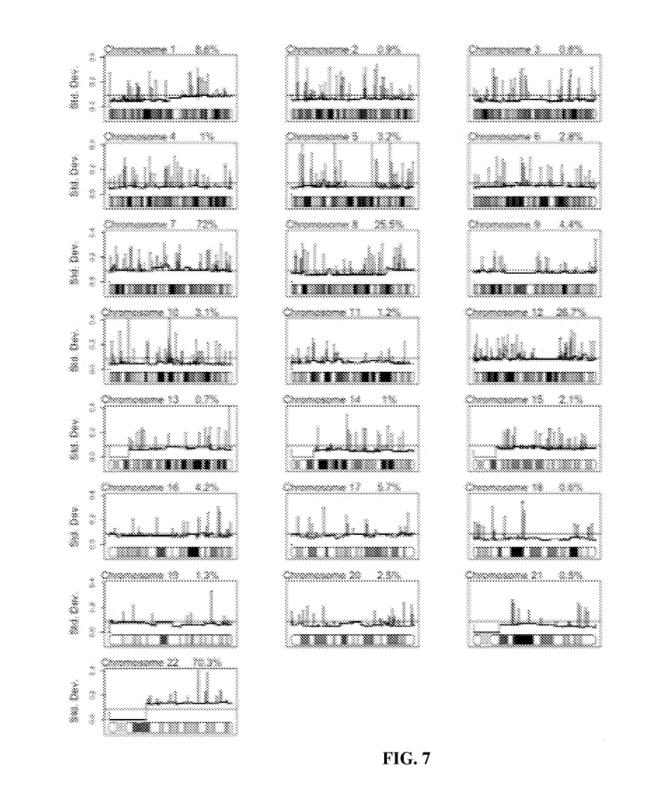

An unsupervised hierarchical cluster analysis of segmented and smoothed copy number estimates for each sample was performed, summarized at 25,000 by intervals, and the 10% of segments with the greatest sample-to-sample variation in copy number were selected. These regions were not evenly distributed throughout the genome, but were concentrated over several chromosomes, most notably 7, 12 and 22, although all chromosomes were represented to some extent, as shown in FIG. 7. The results are shown as a heatmap in FIG. 1, with three clusters standing out. Cluster 1 consists of 7/14 (50%) of the FAs, and 1/12 PTCs screened.

These tumors exhibited a genomic amplification pattern/profile predominantly involving chromosomes 7 and 12, which is consistent with previous studies although the rate observed here is higher than previous estimates (see, e.g., references 8, 12, and 15). Most of the PTCs and FVPTCs clustered together in the center of the heatmap, identified as cluster 2, where few CNVs were observed, which is consistent with the observation that PTCs tend to be relatively stable genomically (see, e.g., references 10 and 16). Finally, in cluster 3, a distinct subset of FVPTCs and FAs were characterized by large deletions in Ch22q, which are indistinguishable from monosomy 22 because of the lack of probes on the acrocentric chromosome 22p arm. Two of the samples with the chromosome 7 and 12 amplifications also harbored this deletion. Upon analysis of clinical and pathological parameters, the Ch22 deletion pattern was found to be associated with younger patients (32 years vs. 46 years, P<0.01, by 2-sided t-test). No other significant associations with clinical indices or specific histopathological features, such as, for example, tumor stage or degree of encapsulation, were observed. All cases showing a BRAF mutation, including 2 cases of FVPTC, were in cluster 2.

### Example 2

### FAs are Enriched for the Presence of Chromosomal Amplifications Relative to FVPTCs and PTCs

Statistical analysis was performed to identify significant CNVs as genomic amplifications and deletions (see, e.g., FIG. 7). The rule for identifying significant CNVs depended on the number of SNPs involved, as well as the magnitude of the copy number change, and was designed to ensure that type I error did not exceed 10%. A total of 464 CNVs were identified as significant genomic aberrations as shown in Table 3A.

TABLE 3A
Detected CNVs in individual thyroid tumor samples.

| ID* | SNP copy number gain | | | | | |
|---|---|---|---|---|---|---|
| Sample | | | | | # SNP | |
| ID* | Cytoband | Start | Stop | Size (bp) | markers | Value |
| S1 | 1p36.13 | 19,705,154 | 19,800,140 | 94,986 | 17 | 0.31 |
| | 1q21.2 | 148,577,451 | 148,638,018 | 60,567 | 5 | 0.49 |
| | 2p11.2 | 88,428,892 | 88,554,147 | 125,255 | 25 | 0.25 |
| | 2q22.2 | 144,504,859 | 144,585,514 | 80,655 | 5 | 0.45 |
| | 2q32.3 | 192,090,179 | 192,100,186 | 10,007 | 6 | 0.42 |
| | 3p25.1 | 12,611,255 | 12,704,485 | 93,230 | 17 | 0.30 |
| | 5q13.1-q13.2 | 68,374,875 | 68,701,565 | 326,690 | 38 | 0.29 |
| | 6p11.1-6q11.1 | 58,822,896 | 62,027,492 | 3,204,596 | 7 | 0.40 |
| | 6q15 | 88,450,677 | 88,576,982 | 126,305 | 22 | 0.26 |
| | 6q21 | 107,562,863 | 107,590,033 | 27,170 | 11 | 0.35 |
| | 7 | 140,736 | 158,812,247 | 158,671,511 | | 0.29 |
| | 9q21.32 | 83,402,356 | 83,405,910 | 3,554 | 4 | 0.52 |
| | 9q34.2 | 135,951,629 | 135,976,732 | 25,103 | 4 | 0.60 |
| | 11p15.4 | 3,662,852 | 3,764,714 | 101,862 | 18 | 0.38 |
| | 11p13 | 33,924,213 | 33,952,308 | 28,095 | 4 | 0.55 |
| | 11p11.12 | 50,508,530 | 51,228,612 | 720,082 | 11 | 0.36 |
| | 12p13.33 | 577,921 | 1,305,458 | 727,537 | 133 | 0.34 |
| | 12p13.31 | 7,668,464 | 8,063,105 | 394,641 | 83 | 0.41 |
| | 12p13.1 | 14,155,049 | 14,648,965 | 493,916 | 68 | 0.35 |
| | 12p12.3 | 19,334,811 | 19,581,151 | 246,340 | 44 | 0.43 |
| | 12p12.1 | 24,933,171 | 25,230,210 | 297,039 | 113 | 0.33 |
| | 12p11.21 | 31,293,957 | 33,013,449 | 1,719,492 | 441 | 0.35 |
| | 12p11.1-q12 | 34,466,271 | 36,743,816 | 2,277,545 | 26 | 0.53 |
| | 12q12 | 39,652,422 | 39,980,210 | 327,788 | 55 | 0.29 |
| | 12q13.13 | 49,016,725 | 50,020,218 | 1,003,493 | 123 | 0.39 |
| | 12q13.2-q13.3 | 55,141,072 | 55,250,997 | 109,925 | 11 | 0.60 |
| | 12q14.2 | 62,868,254 | 63,369,032 | 500,778 | 86 | 0.35 |
| | 12q15 | 69,022,000 | 69,316,000 | 294,000 | 111 | 0.28 |
| | 12q22 | 91,725,146 | 92,472,121 | 746,975 | 135 | 0.31 |
| | 12q22 | 93,730,007 | 94,552,004 | 821,997 | 212 | 0.35 |
| | 12q23.1 | 97,315,513 | 97,468,455 | 152,942 | 32 | 0.26 |
| | 12q23.1 | 97,468,849 | 97,553,430 | 84,581 | 17 | 0.48 |
| | 12q23.1 | 98,915,219 | 99,469,383 | 554,164 | 74 | 0.35 |
| | 12q23.2 | 100,172,485 | 100,926,795 | 754,310 | 171 | 0.34 |
| | 12q24.11-12q24.13 | 107,548,854 | 111,515,857 | 3,967,003 | 352 | 0.28 |
| | 12q24.21-q24.22 | 114,871,593 | 115,733,122 | 861,529 | 176 | 0.28 |
| | 12q24.23 | 116,770,634 | 117,307,617 | 536,983 | 94 | 0.34 |
| | 12q24.23-q24.31 | 118,758,706 | 122,840,427 | 4,081,721 | 481 | 0.30 |
| | 16p13.3 | 1,841,212 | 1,899,620 | 58,408 | 11 | 0.34 |
| | 19p12-q12 | 24,215,273 | 32,848,506 | 8,633,233 | 16 | 0.29 |
| S2 | 4q21.23 | 86,970,408 | 86,975,254 | 4,846 | 5 | 0.42 |
| | 7p22.2-p22.1 | 4,376,280 | 6,903,863 | 2,527,583 | 336 | 0.30 |
| | 7p14.1 | 39,753,634 | 40,299,043 | 545,409 | 49 | 0.36 |
| | 7p12.3 | 47,600,371 | 47,939,559 | 339,188 | 102 | 0.25 |
| | 7p11.2-q11.21 | 55,515,188 | 61,490,330 | 5,975,142 | 200 | 0.26 |
| | 7q11.21 | 61,649,656 | 62,060,344 | 410,688 | 16 | 0.60 |
| | 7q11.21-q21.11 | 62,075,016 | 77,436,474 | 15,361,458 | 1388 | 0.28 |
| | 7q21.3-q22.1 | 97,302,745 | 102,943,265 | 5,640,520 | 658 | 0.28 |
| | 7q22.2 | 104,700,475 | 105,034,706 | 334,231 | 39 | 0.35 |
| | 7q32.1-q32.2 | 127,503,138 | 129,663,252 | 2,160,114 | 324 | 0.25 |
| | 7q36.1 | 151,656,473 | 152,062,784 | 406,311 | 55 | 0.35 |
| | 8q11.1-q11.1 | 43,658,198 | 47,180,142 | 3,521,944 | 31 | 0.41 |
| | 8q11.23-q12.1 | 54,829,907 | 55,617,059 | 787,152 | 135 | 0.26 |
| | 8q12.1 | 56,674,365 | 57,646,989 | 972,624 | 151 | 0.25 |
| | 8q13.3 | 70,925,162 | 71,141,987 | 216,825 | 68 | 0.32 |
| | 8q22.1 | 95,488,331 | 96,320,215 | 831,884 | 181 | 0.27 |
| | 8q22.3 | 103,466,529 | 104,205,125 | 738,596 | 218 | 0.25 |
| | 11q22.3 | 103,334,021 | 103,349,543 | 15,522 | 5 | 0.39 |
| | 12p13.33 | 577,921 | 955,044 | 377,123 | 85 | 0.33 |
| | 12p13.31 | 7,626,398 | 8,039,366 | 412,968 | 89 | 0.35 |
| | 12p13.31 | 8,608,140 | 8,772,935 | 164,795 | 23 | 0.41 |
| | 12p13.2-12p13.1 | 12,051,742 | 13,007,647 | 955,905 | 263 | 0.26 |
| | 12p12.3 | 19,308,616 | 19,662,552 | 353,936 | 68 | 0.32 |
| | 12p11.21 | 31,226,070 | 33,026,317 | 1,800,247 | 464 | 0.27 |
| | 12p11.1-q12 | 34,480,677 | 36,667,312 | 2,186,635 | 21 | 0.49 |
| | 12q13.11 | 45,792,194 | 46,041,641 | 249,447 | 61 | 0.30 |
| | 12q13.11-12q13.13 | 47,312,325 | 50,060,565 | 2,748,240 | 313 | 0.28 |
| | 12q14.2-12q14.3 | 62,893,749 | 63,486,189 | 592,440 | 93 | 0.31 |
| | 12q14.3 | 64,827,573 | 64,847,531 | 19,958 | 4 | 0.96 |
| | 12q23.2 | 100,161,334 | 100,859,758 | 698,424 | 160 | 0.30 |
| | 12q24.23-q24.31 | 118,426,650 | 122,941,163 | 4,514,513 | 555 | 0.27 |
| | 14q21.3 | 43,541,425 | 43,576,977 | 35,552 | 5 | 0.33 |
| | 16q22.1 | 65,467,586 | 69,253,868 | 3,786,282 | 335 | 0.29 |
| | 16q22.3-16q23.1 | 72,710,772 | 74,517,245 | 1,806,473 | 248 | 0.26 |
| | 16q23.2 | 79,656,129 | 80,002,318 | 346,189 | 110 | 0.29 |
| | 20q12 | 39,017,366 | 39,157,752 | 140,386 | 21 | 0.37 |
| | 20q13.12 | 45,147,338 | 45,721,973 | 574,635 | 94 | 0.31 |
| | 20q13.13 | 46,932,762 | 48,042,711 | 1,109,949 | 204 | 0.28 |
| | 20q13.2 | 49,760,837 | 50,187,505 | 426,668 | 130 | 0.36 |
| | 20q13.2 | 51,606,021 | 51,859,114 | 253,093 | 60 | 0.34 |
| S3 | 10p12.31 | 20,890,630 | 20,894,603 | 3,973 | 5 | 2.14 |
| | 12p11.1 | 34,466,271 | 34,564,711 | 98,440 | 4 | 0.84 |
| S4 | 1p36.11 | 27,265,533 | 27,519,669 | 254,136 | 19 | 0.29 |
| | 1p35.3 | 28,436,866 | 29,011,562 | 574,696 | 35 | 0.28 |
| | 1p33 | 47,518,093 | 47,613,179 | 95,086 | 10 | 0.36 |
| | 4p15.2 | 25,140,332 | 25,182,217 | 41,885 | 13 | 0.34 |
| | 6q14.1 | 76,304,232 | 76,473,375 | 169,143 | 16 | 0.28 |
| | 6q23.2 | 134,550,947 | 134,644,147 | 93,200 | 22 | 0.29 |
| | 6q25.1 | 151,519,107 | 151,605,268 | 86,161 | 23 | 0.32 |
| | 7q11.21 | 61,663,407 | 62,172,661 | 509,254 | 23 | 0.38 |
| | 7q33 | 134,754,200 | 134,951,601 | 197,401 | 21 | 0.27 |
| | 8q22.1 | 95,626,728 | 95,643,810 | 17,082 | 7 | 0.45 |
| | 9p13.3 | 33,998,406 | 34,079,395 | 80,989 | 16 | 0.42 |
| | 10p11.1-q11.21 | 39,137,918 | 42,114,131 | 2,976,213 | 9 | 0.50 |
| | 10q24.33 | 104,953,711 | 105,023,005 | 69,294 | 8 | 0.45 |
| | 11p11.2 | 47,425,145 | 47,999,629 | 574,484 | 32 | 0.32 |
| | 12q24.22 | 117,149,206 | 117,167,134 | 17,928 | 4 | 0.64 |
| | 13q32.1 | 94,750,438 | 94,799,350 | 48,912 | 22 | 0.31 |
| | 17p11.2 | 15,945,912 | 16,125,354 | 179,442 | 10 | 0.44 |
| | 17q22 | 54,063,018 | 54,157,457 | 94,439 | 8 | 0.51 |
| | 17q24.2 | 61,637,096 | 61,711,655 | 74,559 | 27 | 0.29 |
| | 17q25.1 | 70,540,347 | 70,956,242 | 415,895 | 48 | 0.25 |
| | 20q13.12 | 45,336,792 | 45,641,776 | 304,984 | 40 | 0.25 |
| | 20q13.31 | 54,560,321 | 54,589,631 | 29,310 | 9 | 0.42 |
| S5 | 2q32.1 | 183,647,418 | 183,672,414 | 24,996 | 4 | 0.42 |
| | 2q32.1 | 183,709,600 | 183,754,364 | 44,764 | 13 | 0.35 |
| | 7p22.3 | 1,618,426 | 1,804,162 | 185,736 | 27 | 0.26 |
| S6 | 7q31.31 | 117,649,478 | 117,661,544 | 12,066 | 4 | 0.78 |
| | 7q36.1 | 151,647,177 | 151,667,867 | 20,690 | 6 | 0.65 |
| | 9q31.1 | 105,618,949 | 105,640,300 | 21,351 | 4 | 0.78 |
| | 12p11.22 | 28,401,743 | 28,435,731 | 33,988 | 6 | 0.72 |
| | 16q12.1 | 45,782,194 | 45,905,281 | 123,087 | 5 | 0.74 |
| S7 | 8p22 | 15,034,440 | 15,038,314 | 3,874 | 5 | 1.04 |
| | 9p21.1 | 29,971,468 | 29,973,603 | 2,135 | 4 | 1.12 |
| | 10q21.1 | 55,088,653 | 55,093,553 | 4,900 | 5 | 0.82 |
| | 11q14.1 | 81,156,560 | 81,158,534 | 1,974 | 5 | 0.68 |
| S8 | normal | | | | | |
| S9 | 2q35 | 219,034,545 | 219,206,172 | 171,627 | 9 | 0.26 |
| | 7q11.21 | 61,649,656 | 61,840,466 | 190,810 | 9 | 0.35 |
| | 9p21.3 | 21,871,338 | 21,910,346 | 39,008 | 5 | 0.44 |
| S10 | 1q25.2 | 177,633,573 | 177,683,970 | 50,397 | 8 | 0.29 |
| | 1q32.3 | 211,052,463 | 211,108,726 | 56,263 | 8 | 0.31 |
| | 2p15 | 61,635,551 | 61,742,206 | 106,655 | 20 | 0.25 |
| | 3p14.3 | 57,665,513 | 57,699,642 | 34,129 | 5 | 0.44 |
| | 4q12 | 57,369,138 | 57,412,952 | 43,814 | 7 | 0.33 |
| | 4q31.3 | 152,187,745 | 152,272,752 | 85,007 | 7 | 0.26 |
| | 5p15.2 | 10,215,790 | 10,716,402 | 500,612 | 118 | 0.25 |
| | 5p15.1 | 16,726,685 | 17,244,616 | 517,931 | 149 | 0.30 |
| | 5p13.3 | 31,715,322 | 32,791,346 | 1,076,024 | 319 | 0.27 |
| | 5p13.1 | 40,907,909 | 40,927,961 | 20,052 | 5 | 0.62 |
| | 5p12 | 42,992,453 | 43,484,078 | 491,625 | 52 | 0.32 |
| | 5p11-q11.1 | 45,938,365 | 49,618,507 | 3,680,142 | 26 | 0.40 |
| | 5q11.2 | 53,786,287 | 53,859,042 | 72,755 | 22 | 0.37 |
| | 5q11.2 | 54,606,995 | 55,634,181 | 1,027,186 | 190 | 0.26 |
| | 5q11.2 | 56,385,031 | 56,563,418 | 178,387 | 15 | 0.42 |
| | 5q12.1 | 59,898,500 | 60,563,277 | 664,777 | 76 | 0.26 |
| | 5q12.1 | 61,476,207 | 61,893,920 | 417,713 | 44 | 0.31 |
| | 5q12.3 | 64,597,201 | 65,409,175 | 811,974 | 133 | 0.26 |
| | 5q13.1 | 67,423,029 | 67,530,747 | 107,718 | 19 | 0.38 |
| | 5q13.1-q13.2 | 68,381,404 | 71,002,933 | 2,621,529 | 68 | 0.39 |
| | 5q14.1 | 79,600,414 | 79,699,756 | 99,342 | 30 | 0.45 |
| | 5q14.1 | 79,700,929 | 80,323,231 | 622,302 | 118 | 0.25 |
| | 5q23.2 | 125,893,989 | 126,211,385 | 317,396 | 64 | 0.39 |
| | 5q31.1 | 130,402,620 | 130,688,294 | 285,674 | 32 | 0.39 |
| | 5q31.1 | 131,836,768 | 132,554,450 | 717,682 | 87 | 0.27 |
| | 5q31.1 | 133,343,957 | 134,268,134 | 924,177 | 102 | 0.28 |
| | 5q31.2 | 137,024,751 | 138,193,116 | 1,168,365 | 101 | 0.30 |
| | 5q31.2-q31.3 | 138,545,384 | 139,103,524 | 558,140 | 35 | 0.35 |
| | 5q32 | 145,542,758 | 145,620,180 | 77,422 | 12 | 0.45 |
| | 5q33.1 | 148,807,387 | 148,969,315 | 161,928 | 35 | 0.33 |
| | 5q33.2 | 153,966,237 | 154,281,664 | 315,427 | 41 | 0.34 |
| | 5q33.3 | 156,190,922 | 156,558,341 | 367,419 | 65 | 0.35 |
| | 5q33.3 | 156,969,197 | 157,337,610 | 368,413 | 79 | 0.32 |
| | 5q33.3 | 159,339,742 | 159,710,846 | 371,104 | 54 | 0.30 |
| | 5q35.2 | 173,807,592 | 174,127,808 | 320,216 | 98 | 0.26 |
| | 5q35.2 | 174,828,792 | 174,997,974 | 169,182 | 47 | 0.28 |
| | 7p22.3 | 1,779,724 | 1,796,425 | 16,701 | 7 | 0.64 |
| | 7p22.2 | 2,266,556 | 2,371,653 | 105,097 | 15 | 0.45 |
| | 7p22.2-p22.1 | 4,435,807 | 6,638,021 | 2,202,214 | 304 | 0.36 |
| | 7p15.3 | 22,773,998 | 24,034,868 | 1,260,870 | 259 | 0.25 |
| | 7p15.2 | 27,218,771 | 27,848,996 | 630,225 | 152 | 0.25 |
| | 7p15.1 | 30,479,684 | 30,639,870 | 160,186 | 25 | 0.34 |
| | 7p14.3 | 32,381,908 | 33,204,725 | 822,817 | 121 | 0.27 |
| | 7p14.1 | 39,838,516 | 40,339,118 | 500,602 | 47 | 0.35 |
| | 7p13 | 44,521,606 | 45,105,688 | 584,082 | 63 | 0.36 |
| | 7p11.2-q11.23 | 55,623,616 | 77,327,719 | 21,704,103 | 1568 | 0.31 |
| | 7q21.3-q22.1 | 97,337,346 | 102,953,131 | 5,615,785 | 657 | 0.31 |
| | 7q22.2 | 104,646,671 | 105,154,749 | 508,078 | 87 | 0.32 |
| | 7q32.1-q32.2 | 127,650,038 | 129,760,286 | 2,110,248 | 321 | 0.28 |
| | 7q32.3 | 130,472,192 | 131,022,872 | 550,680 | 123 | 0.26 |
| | 7q33 | 134,785,342 | 134,969,319 | 183,977 | 20 | 0.47 |
| | 7q34 | 137,367,375 | 138,847,687 | 1,480,312 | 284 | 0.30 |
| | 7q34 | 139,391,271 | 140,564,025 | 1,172,754 | 164 | 0.32 |
| | 7q36.1 | 147,774,349 | 148,695,270 | 920,921 | 164 | 0.29 |
| | 7q36.1-q36.2 | 151,267,242 | 152,653,307 | 1,386,065 | 214 | 0.27 |
| | 7q36.3 | 156,301,895 | 156,943,615 | 641,720 | 117 | 0.30 |
| | 10p13 | 12,358,290 | 12,409,867 | 51,577 | 13 | 0.34 |
| | 10q21.2 | 64,516,847 | 64,549,235 | 32,388 | 9 | 0.30 |
| | 10q26.13 | 126,543,521 | 126,569,148 | 25,627 | 8 | 0.28 |
| | 12 | 64,079 | 132,288,869 | 11,585,055 | 2797 | 0.35 |
| | 14q11.2 | 20,796,924 | 20,855,630 | 58,706 | 11 | 0.27 |
| | 14q13.1-q13.2 | 33,965,728 | 34,186,040 | 220,312 | 27 | 0.26 |
| | 15q22.31 | 63,543,026 | 63,630,207 | 87,181 | 15 | 0.28 |
| | 17p13.3-13.1 | 51,088 | 10,709,171 | 10,658,083 | 2558 | 0.27 |
| | 17p12-q11.2 | 15,370,948 | 28,353,861 | 12,982,913 | 1310 | 0.26 |
| | 17q12-q21.2 | 34,183,104 | 35,710,677 | 1,527,573 | 194 | 0.31 |
| | 17q21.2-q21.31 | 37,010,802 | 40,337,814 | 3,327,012 | 353 | 0.31 |
| | 17q22 | 50,314,685 | 50,327,246 | 12,561 | 13 | 0.41 |
| | 17q22 | 52,449,288 | 52,664,872 | 215,584 | 57 | 0.31 |
| | 17q22-q24.1 | 53,876,128 | 60,541,914 | 6,665,786 | 604 | 0.29 |
| | 17q24.2 | 62,467,382 | 64,290,653 | 1,823,271 | 245 | 0.30 |
| | 17q24.3-q25.1 | 68,283,979 | 69,012,654 | 728,675 | 210 | 0.26 |
| | 17q25.1-q25.2 | 70,469,310 | 72,804,897 | 2,335,587 | 420 | 0.32 |
| | 17q25.3 | 73,628,956 | 74,595,214 | 966,258 | 256 | 0.28 |
| | 17q25.3 | 75,438,157 | 76,221,007 | 782,850 | 157 | 0.26 |
| | 17q25.3 | 77,202,218 | 78,132,403 | 930,185 | 81 | 0.34 |
| | 20p12.3 | 5,480,853 | 5,735,336 | 254,483 | 63 | 0.37 |
| | 20p12.1 | 13,505,267 | 14,014,276 | 509,009 | 98 | 0.26 |
| | 20p12.1-p11.23 | 17,761,094 | 18,157,807 | 396,713 | 107 | 0.28 |
| | 20p11.23 | 19,802,409 | 19,909,094 | 106,685 | 43 | 0.30 |
| | 20p11.21-q11.23 | 25,066,271 | 35,401,507 | 10,335,236 | 657 | 0.30 |
| | 20q13.12-q13.13 | 45,195,959 | 45,925,203 | 729,244 | 162 | 0.30 |
| | 20q13.13 | 46,789,890 | 49,153,010 | 2,363,120 | 449 | 0.27 |
| | 20q13.2 | 49,711,704 | 50,129,256 | 417,552 | 126 | 0.40 |
| | 20q13.2 | 51,570,630 | 51,971,880 | 401,250 | 102 | 0.33 |
| | 20q13.31 | 54,405,028 | 54,765,287 | 360,259 | 90 | 0.31 |
| | 20q13.33 | 61,579,849 | 61,808,066 | 228,217 | 36 | 0.29 |
| S11 | | | | | | |
| S12 | 8q22.1 | 95,697,482 | 95,704,126 | 6,644 | 4 | 1.06 |
| S13 | 9 | 36,587 | 140,147,760 | 140,111,173 | 26866 | 0.34 |
| S14 | 17q12-17q25.3 | 34,634,168 | 78,634,366 | | | |
| S15 | 1q31.1 | 187,316,640 | 187,354,239 | 37,599 | 7 | 0.42 |
| | 1q31.1 | 187,897,346 | 187,997,671 | 100,325 | 26 | 0.31 |
| | 5q11.2 | 54,647,490 | 54,713,276 | 65,786 | 16 | 0.36 |
| | 7q11.21 | 61,681,059 | 62,120,420 | 439,361 | 17 | 0.31 |
| | 9q32 | 115,439,973 | 115,445,389 | 5,416 | 7 | 0.34 |
| | 11p12 | 38,176,864 | 38,357,792 | 180,928 | 35 | 0.26 |
| | 12q13.13 | 49,084,602 | 49,145,087 | 60,485 | 9 | 0.40 |
| | 18q22.1-q22.2 | 64,832,896 | 64,904,521 | 71,625 | 36 | 0.26 |
| S16 | 7p22.3 | 1,775,911 | 1,785,705 | 9,794 | 7 | 0.34 |
| S17 | | | | | | |
| S18 | 6q26 | 163,562,673 | 163,583,227 | 20,554 | 7 | 0.43 |
| | 7q11.21 | 61,649,656 | 61,878,476 | 228,820 | 11 | 0.41 |
| | 11p11.12 | 50,566,118 | 51,249,087 | 682,969 | 10 | 0.33 |
| | 14q11.2 | 21,547,255 | 22,030,942 | 483,687 | 200 | 0.27 |
| | 20q13.2 | 49,892,937 | 49,939,250 | 46,313 | 19 | 0.39 |
| | 21q22.11 | 31,725,269 | 31,749,567 | 24,298 | 8 | 0.47 |
| S19 | 7q11.21 | 61,490,330 | 61,840,466 | 350,136 | 10 | 0.33 |
| S20 | 2q34 | 211,135,486 | 211,197,348 | 61,862 | 8 | 0.32 |
| | 2q35 | 215,550,136 | 215,646,434 | 96,298 | 24 | 0.29 |
| S21 | 1q24.3 | 169,669,291 | 169,715,831 | 46,540 | 15 | 0.27 |
| | 7q11.21 | 61,663,407 | 62,220,970 | 557,563 | 28 | 0.31 |
| | 11p11.12 | 50,470,172 | 51,228,612 | 758,440 | 14 | 0.47 |
| | 12p11.1-q12 | 34,565,140 | 36,751,728 | 2,186,588 | 23 | 0.32 |
| | 19p12-q12 | 24,137,864 | 33,004,040 | 8,866,176 | 36 | 0.25 |
| | 20p11.21 | 24,512,317 | 24,537,790 | 25,473 | 6 | 0.37 |
| S22 | 1p35.2 | 31,293,059 | 31,445,850 | 152,791 | 11 | 0.37 |
| | 1q42.12 | 224,233,178 | 224,617,801 | 384,623 | 51 | 0.27 |
| | 2p21 | 42,570,519 | 42,656,869 | 86,350 | 10 | 0.44 |
| | 3p25.3 | 9,549,327 | 9,709,855 | 160,528 | 19 | 0.26 |
| | 3p22.3 | 32,689,621 | 32,858,600 | 168,979 | 21 | 0.28 |
| | 3q22.3 | 140,035,499 | 140,084,943 | 49,444 | 5 | 0.43 |
| | 7p13 | 43,936,182 | 43,963,600 | 27,418 | 9 | 0.38 |
| | 7q36.1 | 151,752,378 | 151,873,168 | 120,790 | 12 | 0.38 |
| | 8p12 | 30,636,038 | 30,770,877 | 134,839 | 20 | 0.34 |
| | 9p24.1 | 6,655,593 | 6,801,507 | 145,914 | 34 | 0.25 |
| | 12q23.1 | 98,935,297 | 99,019,557 | 84,260 | 14 | 0.28 |
| | 16p12.1 | 22,132,362 | 22,149,769 | 17,407 | 8 | 0.47 |
| | 16q23.2 | 80,329,239 | 80,335,992 | 6,753 | 9 | 0.32 |
| S23 | | | | | | |
| S24 | normal | | | | | |
| S25 | 1q32.1 | 199,477,074 | 199,483,771 | 6,697 | 5 | 0.33 |
| | 13q12.2 | 27,377,963 | 27,464,951 | 86,988 | 15 | 0.31 |
| S26 | | | | | | |
| S27 | normal | | | | | |
| S28 | normal | | | | | |
| S29 | normal | | | | | |
| S30 | | | | | | |
| S31 | 1q32.2-q44 | 206,807,874 | 247,177,330 | 40,369,456 | 8759 | 0.25 |
| S32 | 3q28 | 192,548,086 | 192,552,678 | 4,592 | 6 | 1.96 |
| | 11p15.1 | 16,163,234 | 16,201,098 | 37,864 | 6 | 0.58 |
| | 11p12 | 38,087,375 | 38,129,985 | 42,610 | 4 | 0.68 |
| | 12p13.1 | 13,075,317 | 13,103,493 | 28,176 | 8 | 0.30 |
| | 14q24.3 | 75,217,260 | 75,290,582 | 73,322 | 12 | 0.29 |
| | 14q31.1 | 82,505,512 | 82,528,294 | 22,782 | 10 | 0.46 |
| | 15q24.1 | 71,202,934 | 71,259,940 | 57,006 | 9 | 0.37 |
| S33 | 7 | 140,736 | 158,812,247 | | | 0.34 |
| | 16 | 37,354 | 88,677,423 | 88,640,069 | 16854 | 0.29 |
| S34 | 4p16.3 | 419,720 | 463,952 | 44,232 | 6 | 0.70 |
| | 7p21.3 | 10,825,693 | 10,841,750 | 16,057 | 4 | 1.01 |
| | 11p11.2 | 46,578,968 | 46,632,933 | 53,965 | 5 | 0.70 |
| S35 | 1q31.1 | 187,942,039 | 187,984,282 | 42,243 | 10 | 0.35 |
| | 1q41 | 217,216,616 | 217,222,412 | 5,796 | 4 | 0.61 |
| S36 | 1p11.2-end | 120,982,136 | 247,177,330 | | | 0.34 |
| | 5q35.2 | 175,551,861 | 175,663,413 | 111,552 | 4 | 1.07 |
| | 6q12 | 67,100,918 | 67,101,257 | 339 | 3 | 1.74 |
| | 7p15.2 | 27,210,487 | 27,289,135 | 78,648 | 28 | 0.37 |
| | 8p23.3-p22 | 154,984 | 16,110,852 | 1,031,159 | 545 | 0.29 |
| | 8p11.1-q11.1 | 43,708,547 | 47,388,472 | 3,679,925 | 54 | 0.28 |
| | 18q22.1 | 64,819,792 | 64,846,196 | 26,404 | 11 | 0.78 |
| S37 | | | | | | |
| S38 | | | | | | |
| S39 | 4q13.1 | 64,381,774 | 64,392,223 | 10,449 | 4 | 1.02 |
| | 15q24.1 | 72,673,001 | 72,803,245 | 130,244 | 11 | 0.61 |
| | 22q12.1 | 24,722,234 | 24,725,302 | 3,068 | 4 | 0.92 |

| ID* | SNP copy number loss | | | | | |
|---|---|---|---|---|---|---|
| Sample | | | | | # SNP | |
| ID* | Cytoband | Start | Stop | Size (bp) | markers | Value |
| S1 | 2p21 | 41,871,077 | 41,871,904 | 827 | 4 | −0.76 |
| | 2p14 | 65,125,866 | 65,132,727 | 6,861 | 4 | −0.37 |
| | 3p24.3 | 19,171,481 | 19,242,988 | 71,507 | 12 | −0.43 |
| | 4p15.33 | 15,084,094 | 15,099,656 | 15,562 | 4 | −0.76 |
| | 4q22.1 | 89,006,198 | 89,023,305 | 17,107 | 13 | −0.36 |
| | 4q31.22 | 146,965,285 | 146,966,410 | 1,125 | 4 | −0.90 |
| | 6q23.2 | 132,728,941 | 132,739,275 | 10,334 | 7 | −0.47 |
| | 11q11 | 55,447,013 | 55,465,015 | 18,002 | 19 | −0.36 |
| S2 | 6q26 | 163,408,927 | 163,429,856 | 20929 | 5 | −0.46 |
| | 14q23.1 | 58,516,753 | 58,539,490 | 22737 | 12 | −0.38 |
| | 21q22.3 | 46,815,526 | 46,909,417 | 93891 | 21 | −0.28 |
| S3 | 18q22.3 | 71,271,141 | 71,275,384 | 4243 | 4 | −0.83 |
| S4 | 5q11.1 | 49,907,490 | 49,988,604 | 81114 | 6 | −0.38 |
| | 5q11.2 | 51,773,170 | 51,840,518 | 67348 | 16 | −0.26 |
| | 5q31.1 | 133,183,368 | 133,209,460 | 26092 | 11 | −0.33 |
| | 15q11.2-q26.3 | 18,421,386 | 100,215,583 | 81794197 | 16615 | −0.37 |
| | 17p13.1 | 10,282,051 | 10,337,719 | 55668 | 7 | −0.46 |
| | 22q11.1 | 15,661,931 | 15,823,131 | 161200 | 49 | −0.53 |
| | 22q11.21-q13.33 | 16,644,831 | 49,524,956 | 32880125 | 8142 | −0.95 |
| S5 | | | | | | |
| S6 | 22q11.1-q13.33 | 14,884,399 | 49,524,956 | 34640557 | 8460 | −0.41 |
| S7 | 2q24.3 | 165,567,243 | 165,572,369 | 5126 | 4 | −1.85 |
| | 6p21.31-6qend | 36,515,972 | 170,750,927 | 42503373 | 7537 | −0.27 |
| | 13q | 18,108,426 | 114,121,252 | 96012826 | 20908 | −0.27 |
| S8 | | | | | | |
| S9 | 2q37.1 | 232,877,358 | 232,920,105 | 42747 | 12 | −0.26 |
| | 12q23.3 | 107,098,408 | 107,134,530 | 36122 | 5 | −0.43 |
| | 17q25.3 | 73,605,461 | 73,647,007 | 41546 | 10 | −0.29 |
| S10 | 2q23.1 | 148,933,131 | 148,980,513 | 47382 | 9 | −0.38 |
| | 4p15.2 | 25,009,566 | 25,035,003 | 25437 | 5 | −0.31 |
| | 4q21.23 | 87,056,867 | 87,068,109 | 11242 | 4 | −0.54 |
| | 8q21.13 | 84,443,087 | 84,496,535 | 53448 | 9 | −0.47 |
| | 10q21.3 | 68,359,367 | 68,385,994 | 26627 | 5 | −0.51 |
| | 13q34 | 113,360,001 | 113,491,346 | 131345 | 8 | −0.40 |
| | 15q14 | 34,129,202 | 34,159,437 | 30235 | 12 | −0.37 |
| | 15q26.2 | 92,287,618 | 92,307,865 | 20247 | 4 | −0.53 |
| S11 | 15q13.3 | 29,548,278 | 29,581,222 | 32944 | 8 | −0.30 |
| | 18p11.32 | 2,723,990 | 2,742,837 | 18847 | 4 | −0.54 |
| S12 | | | | | | |
| S13 | | | | | | |
| S14 | 6q14.1 | 79,081,009 | 79,086,086 | 5077 | 5 | −0.69 |
| | 8q23.3-q24.3 | 113,681,735 | 146,245,512 | 32563777 | 7568 | −0.54 |
| S15 | 9q34.3 | 138,419,458 | 138,437,690 | 18232 | 4 | −0.56 |
| | 12q13.13 | 48,548,439 | 48,571,328 | 22889 | 6 | −0.29 |
| | 22q11.21-end | 20,128,907 | 49,524,956 | 29396049 | 7523 | −0.43 |
| S16 | | | | | | |
| S17 | 2q37.1 | 232,039,978 | 232,261,606 | 221628 | 36 | −0.28 |
| | 4p14 | 39,318,327 | 39,490,459 | 172132 | 27 | −0.34 |
| | 4q25 | 113,676,967 | 113,967,887 | 290920 | 38 | −0.26 |
| | 5p15.1 | 15,773,478 | 15,791,017 | 17539 | 5 | −0.33 |
| | 6p22.1 | 27,764,234 | 27,829,814 | 65580 | 18 | −0.34 |
| | 6q13 | 74,098,145 | 74,392,545 | 294400 | 38 | −0.28 |
| | 6q21 | 107,506,663 | 107,610,163 | 103500 | 23 | −0.29 |
| | 10p12.33 | 17,563,047 | 17,616,233 | 53186 | 19 | −0.29 |
| | 10p12.31 | 20,890,630 | 20,894,603 | 3973 | 6 | −3.54 |
| | 10q26.11 | 120,775,453 | 120,947,670 | 172217 | 23 | −0.28 |
| | 11p13 | 34,825,843 | 34,842,993 | 17150 | 8 | −0.36 |
| | 12p13.31 | 7,690,103 | 8,037,956 | 347853 | 79 | −0.30 |
| | 12p13.1 | 14,182,357 | 14,366,359 | 184002 | 31 | −0.25 |
| | 12q23.2 | 100,352,181 | 100,475,974 | 123793 | 32 | −0.26 |
| | 14q22.3 | 54,610,554 | 54,842,289 | 231735 | 24 | −0.27 |
| | 16q21 | 64,438,881 | 64,447,177 | 8296 | 6 | −0.27 |
| | 18q11.2 | 18,861,818 | 18,954,411 | 92593 | 7 | −0.48 |
| | 20p13 | 527,657 | 539,694 | 12037 | 5 | −0.54 |
| | 22q11.23 | 23,781,313 | 23,798,830 | 17517 | 6 | −0.56 |
| S18 | 7q21.13 | 88,231,790 | 88,613,487 | 381697 | 101 | −0.67 |
| | 7q21.13-q21.2 | 90,467,785 | 91,464,889 | 997104 | 150 | −0.49 |
| S19 | | | | | | |
| S20 | 1p32.3 | 52,962,404 | 53,096,080 | 133676 | 11 | −0.45 |
| | 1q32.3 | 211,036,203 | 211,141,648 | 105445 | 21 | −0.33 |
| | 2p23.3 | 25,952,517 | 25,989,756 | 37239 | 6 | −0.62 |
| | 3p24.3 | 21,070,960 | 21,093,365 | 22405 | 4 | −0.94 |
| | 3q29 | 197,643,170 | 197,675,831 | 32661 | 8 | −0.62 |
| | 4q35.2 | 188,023,310 | 188,036,597 | 13287 | 4 | −1.09 |
| | 5q14.1 | 79,600,827 | 79,737,595 | 136768 | 37 | −0.30 |
| | 5q23.1 | 118,819,358 | 118,829,659 | 10301 | 3 | −2.47 |
| | 6q25.1 | 150,008,776 | 150,018,764 | 9988 | 4 | −1.22 |
| | 11q12.3 | 61,502,270 | 61,607,780 | 105510 | 18 | −0.31 |
| | 11q22.3 | 107,175,438 | 107,189,581 | 14143 | 7 | −0.67 |
| | 11q24.3 | 128,420,261 | 128,602,789 | 182528 | 19 | −0.29 |
| | 12p13.33 | 91,464 | 131,131 | 39667 | 15 | 0.26 |
| | 12p13.31 | 7,647,973 | 7,905,308 | 257335 | 62 | −0.25 |
| | 12q12 | 43,585,469 | 43,611,163 | 25694 | 5 | −1.13 |
| | 13q34 | 111,598,206 | 111,601,346 | 3140 | 3 | −4.41 |
| | 14q22.1 | 49,636,675 | 49,654,998 | 18323 | 4 | −1.32 |
| | 19q13.43 | 61,985,643 | 62,012,029 | 26386 | 6 | 0.37 |
| | 20p13 | 3,920,756 | 3,935,738 | 14982 | 4 | −0.76 |
| S21 | 1q42.3 | 232,783,216 | 232,823,041 | 39825 | 9 | −0.35 |
| | 3p24.3 | 18,371,329 | 18,443,527 | 72198 | 17 | −0.28 |
| | 3q26.2 | 172,462,669 | 172,486,498 | 23829 | 11 | −0.38 |
| | 4q28.3 | 135,670,437 | 135,716,639 | 46202 | 11 | −0.38 |
| | 7p14.1 | 42,056,600 | 42,083,380 | 26780 | 16 | −0.26 |
| | 11q21 | 95,675,971 | 95,681,340 | 5369 | 4 | −0.50 |
| | 13q33.1 | 102,043,256 | 102,139,044 | 95788 | 23 | −0.26 |
| | 14q23.1 | 61,030,245 | 61,074,052 | 43807 | 27 | −0.26 |
| S22 | 7q36.3 | 155,370,200 | 155,398,678 | 28478 | 14 | −0.39 |
| | 7q36.3 | 156,017,858 | 156,040,530 | 22672 | 5 | −0.65 |
| | 9p24.1 | 5,172,159 | 5,194,404 | 22245 | 6 | −0.36 |
| | 22q11.1-end | 14,884,399 | 49,524,956 | 34640557 | 8461 | −0.45 |
| S23 | 22q11.1-end | 14,884,399 | 49,524,956 | 34640557 | 8460 | −0.29 |
| S24 | | | | | | |
| S25 | 1p32.1 | 59,141,535 | 59,169,845 | 28310 | 5 | −0.32 |
| S26 | 7q36.1 | 151,524,608 | 151,670,149 | 145541 | 12 | −0.36 |
| | 14q11.2 | 21,760,049 | 21,771,960 | 11911 | 5 | −0.33 |
| S27 | | | | | | |
| S28 | | | | | | |
| S29 | | | | | | |
| S30 | 2p22.2 | 36,969,917 | 37,152,649 | 182732 | 32 | −0.55 |
| S31 | 2q33.1 | 198,308,975 | 198,355,353 | 46378 | 12 | −0.46 |
| | 5q11.2 | 54,660,963 | 54,731,636 | 70673 | 18 | −0.38 |
| | 5q32 | 145,569,735 | 145,616,864 | 47129 | 6 | −0.57 |
| | 5q33.1 | 147,629,374 | 147,696,013 | 66639 | 16 | −0.37 |
| | 6q12 | 65,012,343 | 65,125,363 | 113020 | 22 | −0.36 |
| | 12p11.22 | 28,443,864 | 28,487,596 | 43732 | 10 | −0.53 |
| | 13q12.11 | 18,880,162 | 18,996,553 | 116391 | 5 | −0.87 |
| | 15q23 | 70,102,461 | 70,119,312 | 16851 | 4 | −0.78 |
| | 18q22.1-q22.2 | 64,797,539 | 64,904,585 | 107046 | 52 | −0.26 |
| | 20p11.22 | 21,811,397 | 21,906,049 | 94652 | 24 | −0.39 |
| S32 | 2p15 | 61,512,189 | 61,656,813 | 144624 | 14 | −0.39 |
| | 5q12.2 | 63,565,030 | 63,585,534 | 20504 | 5 | −0.50 |
| | 10q22.1 | 73,610,497 | 73,681,993 | 71496 | 19 | −0.31 |
| | 21q21.3 | 25,924,248 | 25,931,195 | 6947 | 4 | −0.59 |
| S33 | 6q27 | 170,723,055 | 170,750,927 | 27872 | 4 | −0.36 |
| | 9q21.12 | 72,945,733 | 72,948,843 | 3110 | 4 | −1.12 |
| | 19p13.3 | 707,179 | 1,264,763 | 557584 | 94 | −0.37 |
| S34 | | | | | | |
| S35 | 6p21.1 | 44,504,079 | 44,515,875 | 11796 | 5 | −0.43 |
| S36 | 4q28.1 | 125,566,164 | 125,599,159 | 32995 | 4 | −1.16 |
| | 4q28.3 | 138,568,314 | 138,574,552 | 6238 | 4 | −1.15 |
| | 11p12 | 38,334,468 | 38,363,752 | 29284 | 8 | −0.87 |
| | 13q31.3 | 89,310,343 | 89,314,035 | 3692 | 4 | −1.40 |
| | 15q21.3 | 54,272,890 | 54,283,874 | 10984 | 5 | −1.08 |
| S37 | 1q22 | 153,681,392 | 154,169,010 | 487618 | 30 | −0.29 |
| | 3p25.1 | 12,630,689 | 12,772,747 | 142058 | 23 | −0.27 |
| | 3q26.32 | 178,325,169 | 178,539,833 | 214664 | 20 | −0.27 |
| | 3q26.33 | 182,042,024 | 182,133,656 | 91632 | 15 | −0.26 |
| | 4q14 | 39,266,759 | 39,845,819 | 579060 | 76 | −0.26 |
| | 5p13.2 | 37,065,642 | 37,405,715 | 340073 | 23 | −0.28 |
| | 5q32 | 145,602,665 | 145,623,118 | 20453 | 6 | −0.51 |
| | 6q21 | 107,398,729 | 107,666,031 | 267302 | 59 | −0.25 |
| | 7p11.2 | 55,991,781 | 56,011,943 | 20162 | 4 | −0.73 |
| | 7q11.23 | 75,031,499 | 75,326,974 | 295475 | 56 | −0.33 |
| | 7q36.1 | 151,141,670 | 151,148,075 | 6405 | 6 | 0.41 |
| | 10p14 | 12,019,008 | 12,255,186 | 236178 | 31 | −0.30 |
| | 10q23.33 | 97,411,335 | 97,441,508 | 30173 | 5 | −0.53 |
| | 14q13.1-q13.2 | 34,003,561 | 34,494,187 | 490626 | 81 | −0.28 |
| | 14q31.1 | 79,608,285 | 79,635,167 | 26882 | 7 | 0.41 |
| | 15q21.2 | 50,033,957 | 50,164,332 | 130375 | 12 | −0.37 |
| | 15q21.3 | 53,441,704 | 53,681,850 | 240146 | 34 | −0.28 |
| | 15q25.2-q25.3 | 82,920,090 | 83,103,377 | 183287 | 20 | −0.29 |
| | 16q12.1 | 48,592,181 | 48,800,875 | 208694 | 20 | −0.33 |
| | 18p11.31 | 3,335,173 | 3,415,211 | 80038 | 25 | −0.26 |
| | 18p11.21 | 12,721,854 | 12,726,556 | 4702 | 4 | −0.52 |
| | 18q11.2 | 21,993,023 | 22,190,589 | 197566 | 24 | −0.25 |
| | 20q13.2 | 49,921,745 | 50,139,810 | 218065 | 73 | −0.26 |
| S38 | 2q13 | 111,623,233 | 111,726,957 | 103724 | 30 | −0.58 |
| | 2q36.1 | 221,767,011 | 221,968,993 | 201982 | 61 | −0.62 |
| | 7q11.22 | 67,333,089 | 67,559,377 | 226288 | 45 | −0.53 |
| | 7q34 | 140,145,576 | 140,174,786 | 29210 | 5 | −0.64 |
| S39 | 3q28 | 192,465,170 | 192,488,918 | 23748 | 6 | −0.88 |
| | 5p11 | 45,817,629 | 45,832,303 | 14674 | 4 | −1.03 |
| | 6q25.1 | 150,007,433 | 150,046,472 | 39039 | 7 | −0.69 |
| | 9q34.3 | 139,876,646 | 139,986,010 | 109364 | 6 | −0.94 |
| | 22q12.3 | 31,748,564 | 31,761,164 | 12600 | 5 | −0.85 |

*S1-S14 were FAs; S15-S27 were FVPTCs; S28-S39 were PTCs.

Chromosomal amplifications were more frequent in FAs than in FVPTCs or in PTCs (P<0.01, Chi-square test, see, e.g., FIG. 2), occurring in ≧3 FAs at 7p, 7q, 12p, 12q, 17q and 20q13.12. In PTCs, an amplification of 1q41 region occurred in 3/12 samples; and a deletion of 5q32 occurred in 2 samples. In FVPTCs, 7p11.21 was amplified in 4/13 samples; and deletions at 12p13.31 and the whole arm of 22q were also common.

### Example 3

### Sets of 5-50 Copy Number Variant Genes Accurately Distinguish Benign FAs from Malignant FVPTCs and PTCs

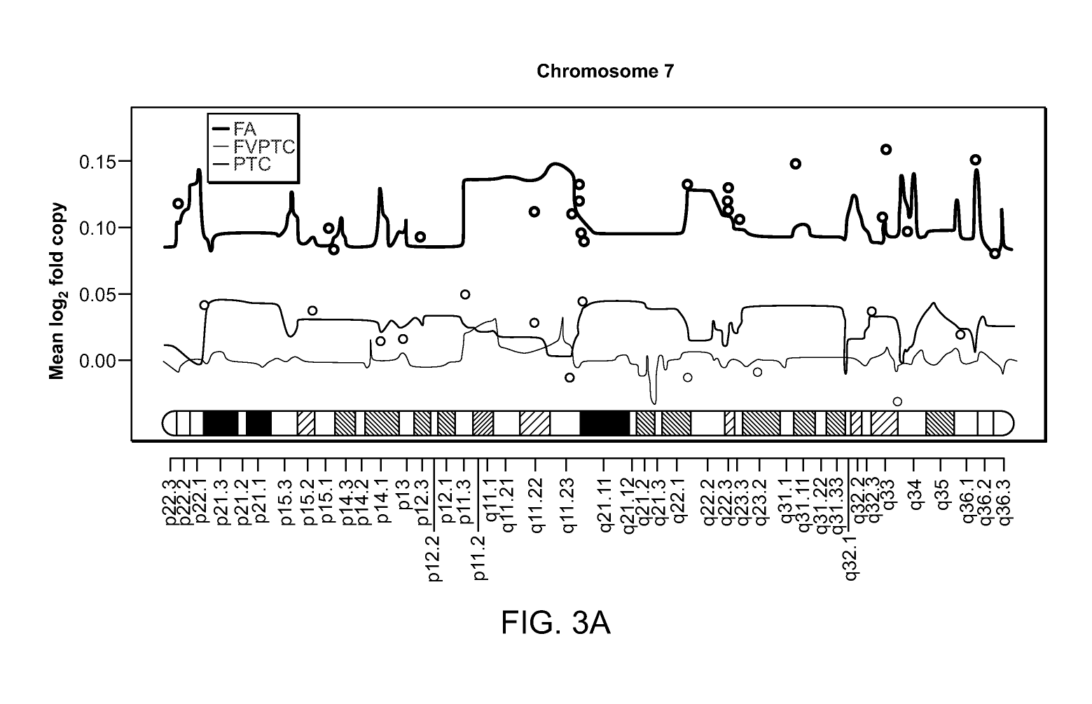

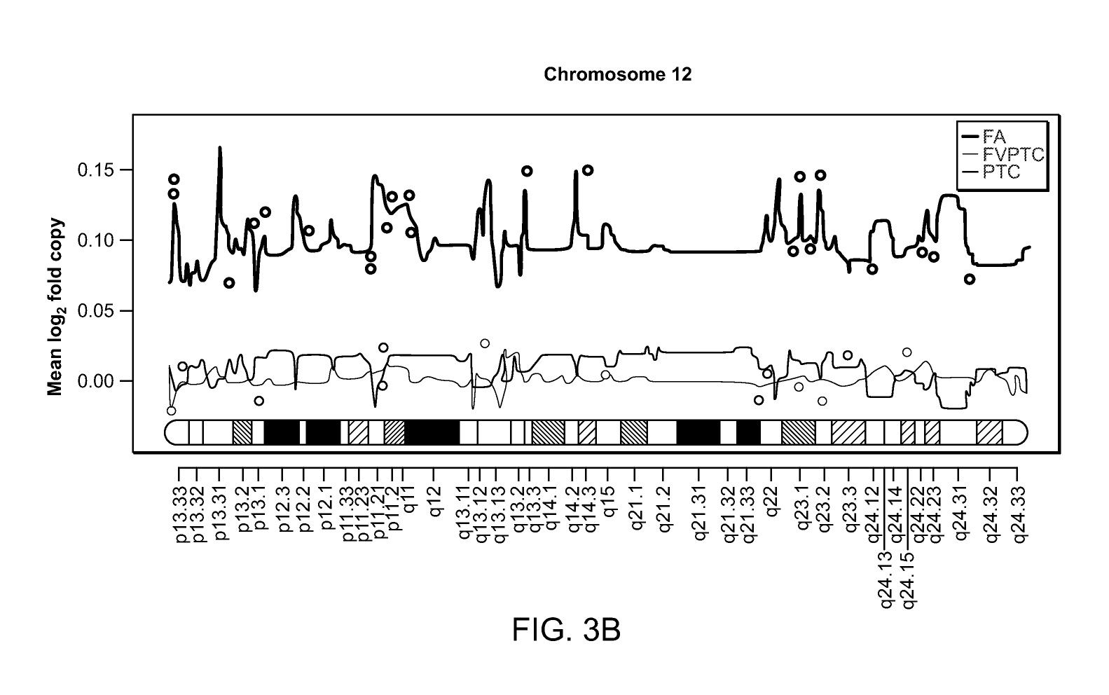

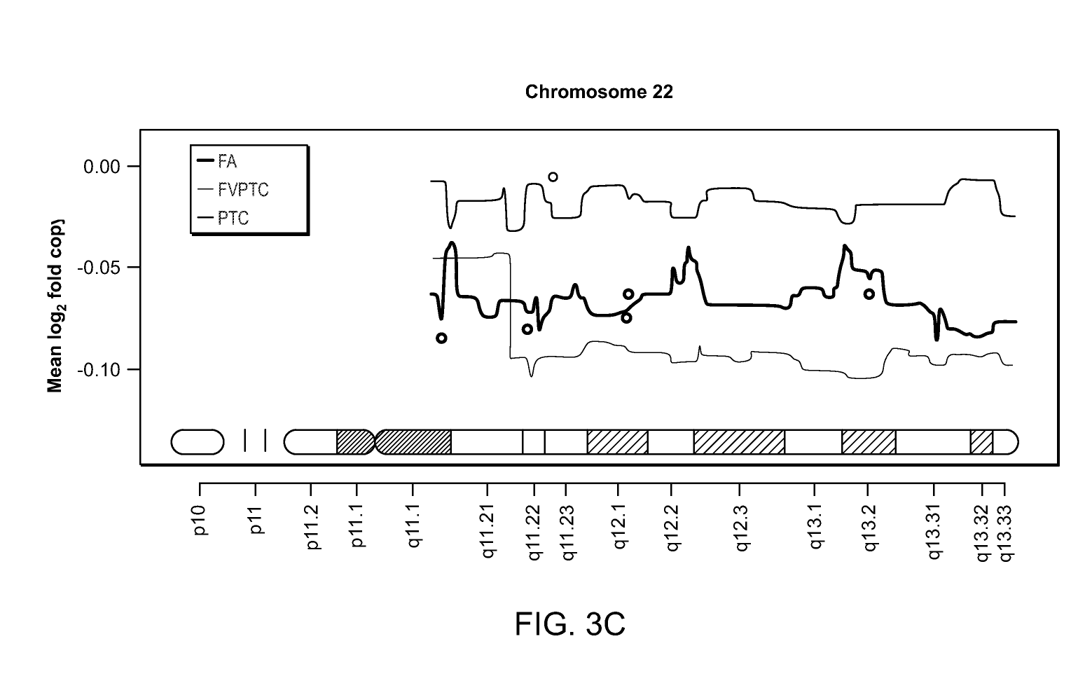

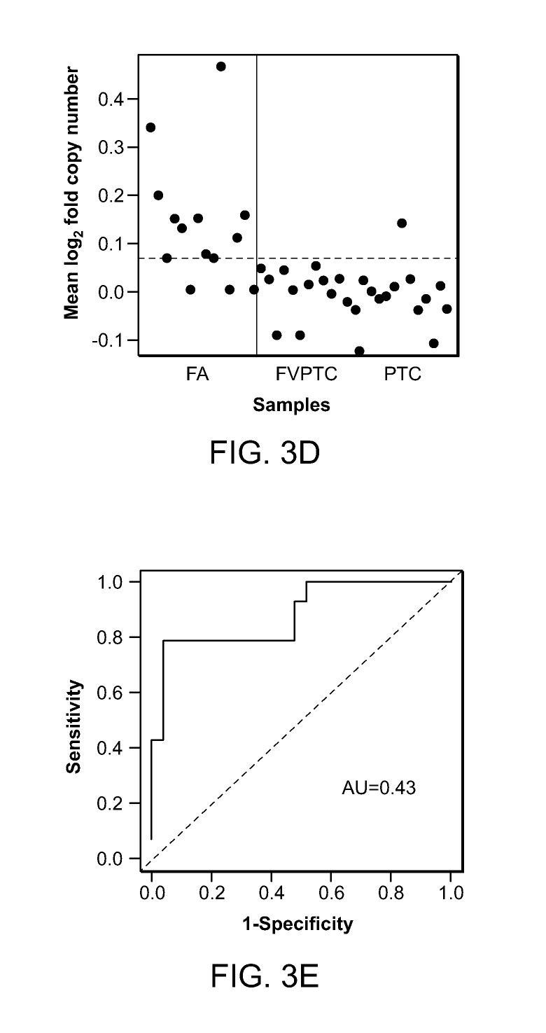

To identify genes in which copy number differed by tumor type, the original segmented data was mapped to genes and analyzed by an ANOVA, and the Type I error was controlled by the Benjamini-Hochberg false discovery rate and maintained at a level less than 10%. A total of 1209 genes for which DNA copy number showed significant differences (adjusted P<0.05) between FAs and FVPTCs/PTCs were found. The majority of these genes were located on chromosomes 7, 12, and 17. The dominant CNV pattern was determined to be low level but widespread copy number gain of Ch12 in FAs, as illustrated in FIG. 3A-C, which show the mean fold changes across all samples on Ch7, Ch12, and Ch22, separated by tumor subtype.

To obtain a gene set whose CNVs could distinguish benign FAs from malignant PTCs and FVPTCs, the top 10 ranked genes on Ch12 were selected, ordered according to their statistical significances, and their mean copy number changes within each sample were calculated. This resulted in a significant difference in mean copy number change (P<0.001). Discrimination between classes (e.g., FAs, PTCs, and FVPTCs) was optimal at a cutoff of 0.07 for mean log fold copy number change. A 10-gene set, including, for example, the genes NADH dehydrogenase (ubiquinone) 1 alpha subcomplex, 12 (NDUFA12), nuclear receptor subfamily 2, group C, member 1 (NR2C1), FYVE, RhoGEF and PH domain containing 6 (FGD6), vezatin, adherens junctions transmembrane protein (VEZT), microRNA 331 (MIR331), ribosomal protein L29 pseudogene 26, hypothetical protein LOC729457, methionyl aminopeptidase 2 (METAP2), ubiquitin specific peptidase 44 (USP44), and CD163 molecule-like 1 (CD163L1), was identified that could accurately classify 11 out of 14 FAs and 24 out of 25 PTCs and FVPTCs (see, e.g., FIG. 3D). To evaluate the performance of this particular gene set in classifying different tumor types, a receiver operating characteristic (ROC) analysis was applied to this 10-gene set, which resulted in an area under the ROC curve (AUC) of 0.88 (FIG. 3E). This result was confirmed by leave-one-out cross-validation, which accurately classified 10 of 14 FAs and 23 of 25 PTCs/FVPTCs, with an AUC of 0.84, using the same cutoff of 0.07. Results were not sensitive to the number of genes used, remaining stable from 5 genes (AUC=0.85) to at least 50 genes (AUC=0.82); consequently, sets of between about 5 and 50 CNV genes provide accurate, FA or PTC/FVPTC specific diagnostic ability. For example, a 50 gene super set of CNV markers may include the 50 genes listed in Table 3B.

TABLE 3B

| | | Accession |
|---|---|---|
| geneSymbol | geneDescription | Number |
| NDUFA12 | NADH dehydrogenase (ubiquinone) | NM_001258338 |
| | 1 alpha subcomplex, 12 | |
| NR2C1 | nuclear receptor subfamily 2, | NM_001032287 |
| | group C, member 1 | |
| FGD6 | FYVE, RhoGEF and PH domain | NM_018351 |
| | containing 6 | |
| VEZT | vezatin, adherens junctions | NM_017599 |
| | transmembrane protein | |
| MIR331 | microRNA 331 | NR_029895 |
| RPL29P26 | ribosomal protein L29 pseudogene 26 | NC_000012.11 |
| LOC729457 | hypothetical protein LOC729457 | NC_000012.10 |
| METAP2 | methionyl aminopeptidase 2 | NM_006838 |
| USP44 | ubiquitin specific peptidase 44 | NM_001042403 |
| CD163L1 | CD163 molecule-like 1 | NM_174941 |
| LOC727815 | hypothetical LOC727815 | NC_000012.10 |
| BICD1 | bicaudal D homolog 1 (Drosophila) | NM_001003398 |
| FGD4 | FYVE, RhoGEF and PH domain | NM_139241 |
| | containing 4 | |
| DNM1L | dynamin 1-like | NM_005690 |
| YARS2 | tyrosyl-tRNA synthetase 2, | NM_001040436 |
| | mitochondrial | |
| UTP20 | UTP20, small subunit (SSU) | NM_014503 |
| | processome component, homolog | |
| | (yeast) | |
| ARL1 | ADP-ribosylation factor-like 1 | NM_001177 |
| SPIC | Spi-C transcription factor | NM_152323 |
| | (Spi-1/PU.1 related) | |
| WNK1 | WNK lysine deficient protein | NM_001184985 |
| | kinase 1 | |
| DRAM | DNA-damage regulated autophagy | NM_018370 |
| | modulator 1 | |
| RAD52 | RAD52 homolog (S. cerevisiae) | NM_134424 |
| HSPD1P12 | heat shock 60 kDa protein 1 | NC_000012.11 |
| | (chaperonin) pseudogene 12 | |
| CERS5 | ceramide synthase 5 | NM_147190 |
| LIMA1 | LIM domain and actin binding 1 | NM_001113546 |
| MYBPC1 | myosin binding protein C, slow type | NM_001254718 |
| CHPT1 | choline phosphotransferase 1 | NM_020244 |
| SYCP3 | synaptonemal complex protein 3 | NM_001177948 |
| PKP2 | plakophilin 2 | NM_001005242 |
| CCDC53 | coiled-coil domain containing 53 | NM_016053 |
| HAUS6 | HAUS augmin-like complex, subunit 6 | NM_001270890 |
| LOC729925 | hypothetical protein LOC729925 | NC_000009.10 |
| YPEL2 | yippee-like 2 (Drosophila) | NM_001005404 |
| DHX40 | DEAH (Asp-Glu-Ala-His) box | NM_001166301 |
| | polypeptide 40 | |
| CLTC | clathrin, heavy chain (Hc) | NM_004859 |
| PTRH2 | peptidyl-tRNA hydrolase 2 | NM_016077 |
| TMEM49 | vacuole membrane protein 1 | NM_030938 |
| MIR21 | microRNA 21 | NR_029493 |
| TUBD1 | tubulin, delta 1 | NM_001193609 |
| PLIN2 | NADH dehydrogenase (ubiquinone) | NC_000017.10 |
| | 1 beta subcomplex, 8, pseudogene 2 | |
| RPS6KB1 | ribosomal protein S6 kinase, 70 kDa, | NM_003161 |
| | polypeptide 1 | |
| HEATR6 | HEAT repeat containing 6 | NM_022070 |
| LOC645638 | WDNM1-like pseudogene | NC_018928.1 |
| LOC653653 | adaptor-related protein complex 1, | NC_000017.10 |
| | sigma 2 subunit pseudogene | |
| LOC650609 | similar to Double C2-like | NC_000017.9 |
| | domain-containing protein beta | |
| | (Doc2-beta) | |
| CA4 | carbonic anhydrase IV | NM_000717 |
| USP32 | ubiquitin specific peptidase 32 | NM_032582 |
| SCARNA20 | small Cajal body-specific RNA 20 | NR_002999.2 |
| C17orf64 | chromosome 17 open reading frame 64 | NM_181707 |
| APPBP2 | amyloid beta precursor protein | NM_006380 |
| | (cytoplasmic tail) binding protein 2 | |

The chromosome 12 copy number changes were validated in order to: 1) provide a technical validation of the Ch12 signature using an independent, PCR-based assay; and 2) investigate if the CNV-signature found in FAs was in fact FA-specific, or also present in FCs/HCs and FVPTCs on the one hand, or in ANs on the other, given the morphological similarities between these follicular neoplasms. The genes NDUFA12, NR2C1, FGD6, VEZT (the top 4 ranked genes according to their statistical significance by ANOVA) and GDF3 (located at 12p13.31, a region showing amplifications in FAs and deletions in FVPTCs) were selected for validation, and the average copy number levels across the five genes was used to obtain a single estimated value for each sample. The Genbank annotation for these five genes can be found in Table 4.

TABLE 4
Genbank annotation information of 5 Chromosome
12 genes used for validation

| Gene | Gene | | | Adj. |
|---|---|---|---|---|
| symbol | ID | Cytoband | Gene Name | P value* |
| NDUFA12 | 55967 | 12q22 | NADH dehydrogenase | 0.047 |
| | | | (ubiquinone) 1 | |
| | | | alpha subcomplex, 12 | |
| NR2C1 | 7181 | 12q22 | nuclear receptor | 0.047 |
| | | | subfamily 2, | |
| | | | group C, member 1 | |
| FGD6 | 55785 | 12q22 | FYVE, RhoGEF and PH | 0.047 |
| | | | domain containing 6 | |
| VEZT | 55591 | 12q22 | vezatin, adherens | 0.047 |
| | | | junctions transmembrane | |
| | | | protein | |
| GDF3 | 9573 | 12p13.31 | growth differentiation | 0.048 |
| | | | factor 3 | |

*Empirical Bayes modified ANOVA analysis (FA vs PTC/FVPTC).

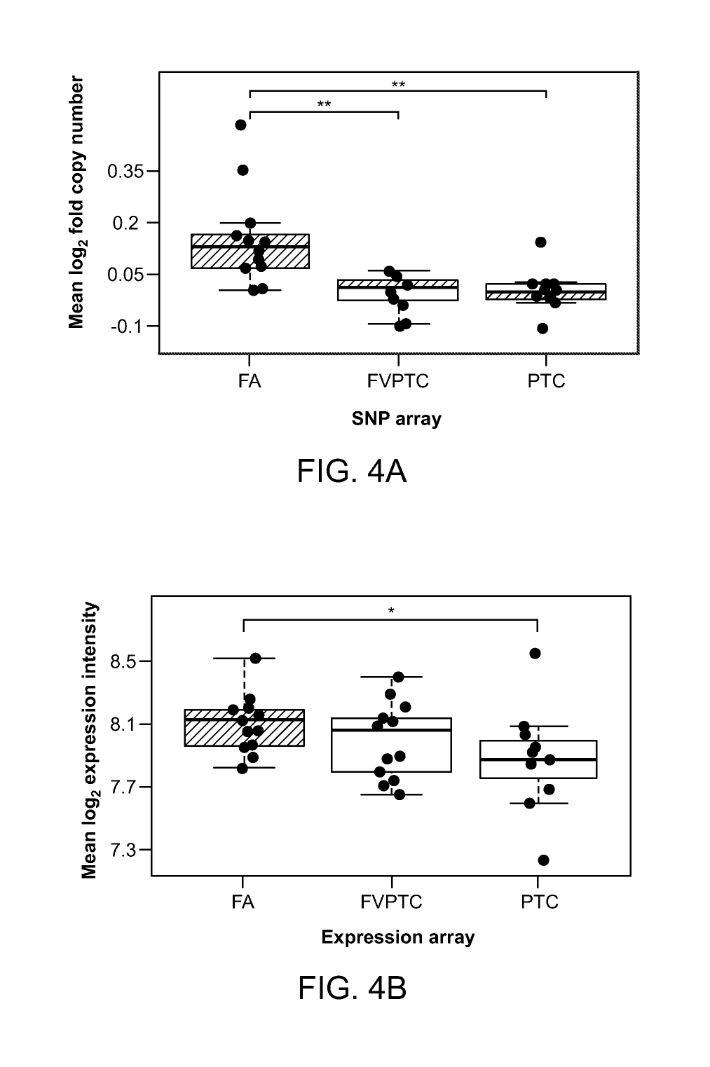

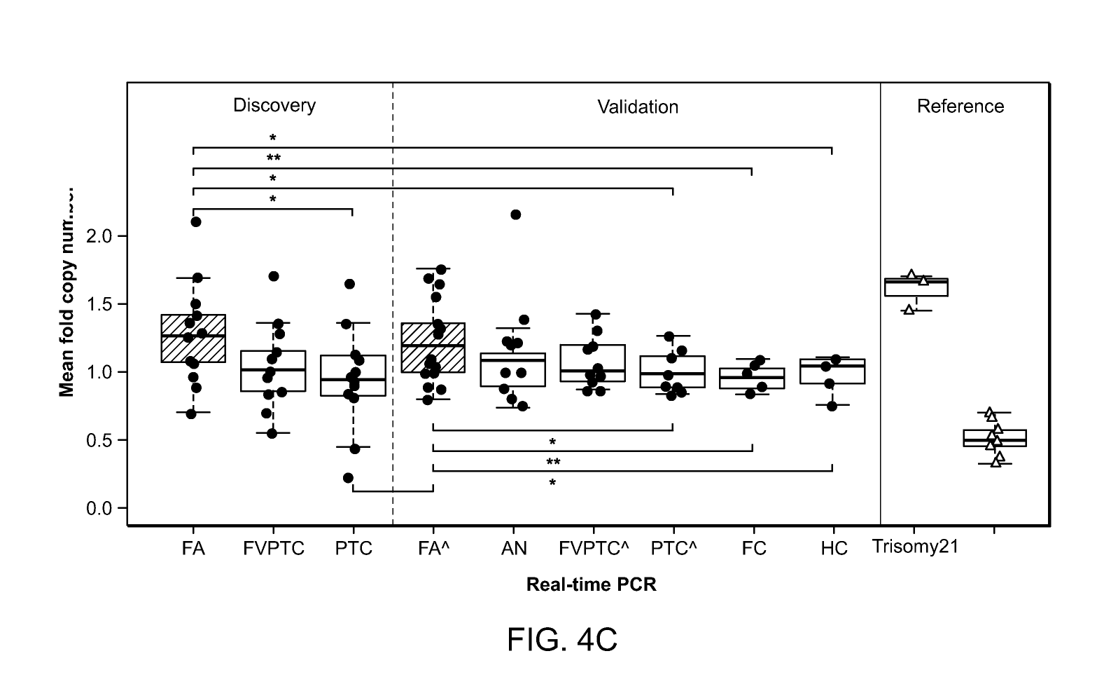

Based on the distributions of the five gene score in benign and malignant tumors on the SNP array (see, e.g., FIG. 4A), a power analysis was performed. The power analysis indicated that about 18 additional FAs and 18 PTC/FVPTCs would be required to have a 90% likelihood of detecting a difference in chromosome 12 amplification in an independent validation sample. The quantitative real-time PCR analysis of copy number changes for these 5 genes independently confirmed our SNP array finding that FAs most frequently harbor Ch12 amplifications, both in the original 39 tumors (see, e.g., FIG. 4C), as well as in an independent test set of 18 FAs and 19 malignant tumors, including 9 PTCs and 10 FVPTCs. Twelve ANs and 12 samples from additional malignant tumor subtypes (7 FCs and 5 HCs) were also tested. While a small number of ANs showed elevated Ch12 CNV scores, both FCs and HCs did not. The gene expression array analysis of these 39 thyroid tumors (see methods section below) also showed that the average expression level of these 5 genes presented the same trend, confirming the above described results on a complementary assay platform (see, e.g., FIG. 4B).

### Example 4

### Detection of Chromosome 12 Amplification Signature Provides an Accurate Diagnostic for FAs in Matched FNA Samples

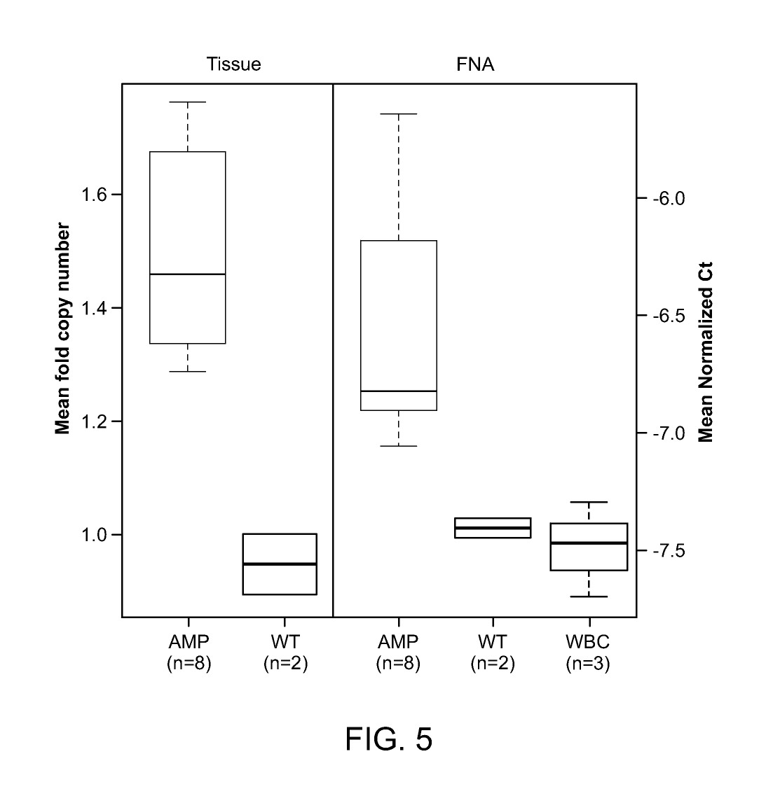

In order to determine the clinical applicability of detecting CNVs in thyroid FNA samples, given the expected contamination with blood and white blood cells (WBCs), a small FNA feasibility study was performed. Matching FNAs were available from 18 of the FA cases considered under the present study. All FNA samples were obtained intraoperatively after surgical isolation of the target lesion and stored in 95% ethanol. FNA samples were enriched for epithelial cells using magnetic beads, resulting in a total of 10 matching FNA samples with detectable amounts of DNA, as determined by achieving identifiable real-time PCR threshold cycle numbers. The results of the successful QPCR assays of this subset are shown in FIG. 5. The samples were plotted separately based on their amplification status as determined by the tissue-based assays. The results clearly indicate that the Ch12 amplification signature is detectable and distinguishable from WT in thyroid FNA-derived DNA, as long as sufficient epithelial cells are present in the sample.

The somatic genomic alterations in one benign (FAs) and two malignant (PTC and FVPTC) thyroid tumor subtypes were characterized. These three tumor subtypes were the focus of the analysis because they are the most commonly associated with a suspicious but inconclusive preoperative cytopathology. The much more limited FC samples were reserved for a validation of the screening results. In total, 39 thyroid tumor/normal pairs, including 14 FAs, 13 FVPTCs, and 12 PTCs, were analyzed using the Illumina 550K SNP Array platform. This is believed to be the first study to report genome-wide DNA copy number profiles comparing FA, PTC and FVPTC thyroid tumors based on a high-resolution SNP array analysis.

The most frequent genomic aberrations occurred in FAs, and included amplifications of chromosomes 7 and 12, which is consistent with prior CGH and array-CGH studies (see, e.g., references 8, 12, 15). Importantly, the frequency of such events in FAs as determined in the present study is much higher than previously estimated using lower resolution techniques. Conversely, with the notable exception of Ch22 deletions observed in several FVPTCs, both PTCs and FVPTCs showed relatively few copy number changes. This is consistent with the notion that these are relatively stable, from a genomic standpoint, neoplasms at least in their initial, well differentiated stages (see, e.g., references 10, 14, 16,).

The unsupervised hierarchical cluster analysis of detected CNVs clearly shows distinct patterns, which are identified in FIG. 1 as clusters 1, 2, and 3. The consistent CNV patterns in cluster 1 found in many FAs on chromosomes 7 and 12 suggest that FAs showing these changes may represent a subset that may harbor a developmental potential that differs from that of structurally more stable FAs. Furthermore, since Ch12 amplifications were not identified in malignant tumor subtypes, this could indicate that FAs harboring this cluster 1 CNV signature are unlikely to progress (e.g., they may not be precursor lesions), in contrast to FAs showing Ch22 deletions, as discussed further below. Because follicular neoplasms reflect a spectrum of disease with considerable morphological overlap, rather than discreet entities, and the malignant potential of early stage FVPTCs is often unclear and not always easily distinguishable from other follicular neoplasms (see, e.g., references 21, 26), that the presently described CNV patterns may provide diagnostic capabilities to help identify subsets of follicular neoplasms with different biological potential.

Although the number of cases showing Ch22 deletions is small, the consistency of the Ch22deletion patterns seen in several FAs and FVPTCs suggests that this genetic lesion may also represent a distinct subset of these tumors. In this context, it is worth noting that large Ch22 deletions and monosomy 22 have been associated with subsets of malignant follicular neoplasms (see. e.g., references 27, 28), and may therefore be indicative of precursor lesions. However, with the exception of a statistically significant association of the Ch22 deletion cluster with younger age, there was no apparent correlation of any clinical or pathological parameter with a particular CNV cluster. Of note, the 2 FVPTCs harboring BRAF mutations were in the PTC-associated cluster 2, supporting the notion that FVPTCs may broadly belong to either follicular or papillary tumors, each with its distinct molecular and clinical signatures.

The most striking result of the present study arose from a gene-by-gene comparison of copy number in the 14 benign and 25 malignant lesions of the discovery cohort. As seen in the cluster analysis in FIG. 1, as many as 50% of the FAs showed distinctive amplification of chromosomes 7 and 12. In particular, the panel of the top 10 genes (e.g., NDUFA12, NR2C1, FGD6, VEZT, MIR331, RPL29P26, LOC729457, METAP2, USP44, CD163L1) showing significant copy number changes by ANOVA could distinguish FAs and PTC/FVPTCs in all but 4 out of 39 cases. The estimated copy numbers, although elevated, were moderate, suggesting that not all adenoma cells harbor a detectable copy number change, reflecting intra-tumor heterogeneity. The stromal component of well-differentiated thyroid tumors is typically minor, and is therefore unlikely to strongly affect CNV patterns.

To confirm this result by independent methodologies, five genes, NDUFA12, NR2C1, FGD6, VEZT and GDF3, were selected for validation using quantitative Real-time genomic PCR (QPCR). The gene expression array data for the same samples was also analyzed to determine if the amplification on Ch12 could be detected by such an approach as well. Both copy number changes, as assessed by QPCR, and gene expression, as assessed by transcriptome array, supported the presence of gene amplifications on Ch12 in FAs. In addition, a number of genes identified in an integrated analysis of gene expression and DNA copy number showed concordant results between DNA copy number change and gene expression levels (e.g., the above described 50 gene superset). Not surprisingly, Ch12 was over-represented in this set, but similar results were observed in other regions as well.

Ch12 copy number changes were also confirmed in an independent test cohort that included both benign and malignant tumors, which again showed amplification in FAs, while other tumor subtypes, regardless of dignity (e.g., tumor dignity means malignant versus benign) or presence or absence of oncocytic cells, generally did not. This suggests that FAs with amplifications on Ch12 are less likely to progress to thyroid cancer, since that genetic change would not be expected to disappear as FAs progressed. Accordingly, the present disclosure may provide the ability to positively identify FAs with a low chance of malignant progression, which would be an important adjunct to our current set of diagnostic tests that are focused on identifying oncogenic mutations and translocations in malignant thyroid tumors.

In light of these results, tumor pathology was assessed to determine if any distinct morphological patterns matching the Ch12 CNVs could be identified. Both initial blinded and subsequent open reviews failed to identify a morphological subset in our FA cohort. It is also noteworthy that among our samples in the morphological continuum ranging from AN to FA to FVPTC, small numbers of both ANs and FVPTCs harbored the Ch12 amplification characteristic of FAs, which may support a reevaluation of these lesions based on molecular traits in addition to morphological characteristics. It remains to be seen if the 5 genes that we used to represent chromosome 12 have any functional roles in thyroid tissues or thyroid neoplasia, since they were selected based on the structural chromosomal changes detected by the above described CNV analysis.

Finally, an initial feasibility study was performed to determine the Ch12 amplification signature could be detected in cytological specimens. The principal challenge in applying the above described quantitative genomic PCR assay to FNA samples is the unavoidable presence of varying amounts of blood contamination. To address this challenge, the archival FNA samples were fractionated using a commercially available magnetic bead separation approach, and the epithelial cell enrichment lead to the correct classification of all 10 amplifiable DNA preparations, as shown in FIG. 5. Of note, the magnetic bead separation was successful on archival FNA samples preserved in 95% ethanol for several years, and it is likely that yields may improve if the separation is performed on freshly obtained FNA material.

In summary, the present disclosure provides a high-resolution analysis of somatic copy number aberrations in FA, PTC and FVPTC thyroid tumors. According to the techniques herein, distinct genomic patterns of copy number changes associated with benign and malignant thyroid tumors, of which the gene copy number gains in Ch12 were the most distinctive, were limited to benign tumors. These amplifications were verified using Realtime-PCR of genomic DNA and transcriptome arrays of the same 39 tumor-normal paired thyroid samples, and the specificity of this result was validated on an additional independent test set of benign and malignant thyroid tumors. The results demonstrated the diagnostic feasibility of assessing CNV signatures in thyroid FNA samples.

Since FAs are a common source of inconclusive pre-operative cytopathology results, the techniques herein, which provide a molecular signature (e.g., Ch12 amplifications) that positively identifies a subset of follicular neoplasms with no malignant potential, represents an important diagnostic adjunct to the currently available tests for oncogenic genetic changes in thyroid cancers. Similarly, the ability to identify the presence of Ch22 deletions in FAs is a useful diagnostic indicative of a premalignant state that may ultimately lead to invasive disease. The present disclosure illustrates the value of the molecular characterization of benign thyroid tumors and well-differentiated thyroid cancer, which continue to confound the pre-operative diagnosis of thyroid nodules, and may help justify the clinical development of molecular assays based on an epithelial cell-enriched fraction of the standard FNA sample.

The results described herein above were obtained using the following methods and materials.

Tissue Samples and DNA Isolation:

Cases were identified that underwent partial or complete thyroidectomy for malignant or indeterminate thyroid lesions at the Johns Hopkins Medical Institutions between 2000 and 2008 and from whom tissue had been immediately snap frozen in liquid nitrogen within one hour of surgery and stored at −80° C. until use. Initial case selection was based on review of the official surgical pathology reports identifying thyroid tumor subtypes falling into the scope of this study. Cases were then selected for availability of adequate matching tumor and normal tissue and passing quality controls for both DNA and RNA. The study pathologist (WW) reviewed both the official archival permanent H&E sections to confirm the original diagnoses as well as the research cryosections to confirm tumor content of the analyzed sample. The diagnoses of thyroid tumors in this study was based on the criteria described in the 2004 World Health Organization (WHO) monograph on endocrine tumors (see, e.g., reference 29). None of these cases had oncocytic features. Each tumor tissue block used for nucleic acid isolation was confirmed to contain more than 70% tumor cells on H&E-stained cryosections (see, e.g., reference 30).

SNP Array Analyses:

DNA from 39 thyroid tumor-normal paired samples was genotyped using the Illumina 550K SNP Array (Illumina, San Diego, Calif.). DNA samples were assessed for quality both by NanoDrop Spectrophotometry and agarose gel electrophoresis. Samples judged to be of sufficient quality were assayed at the Center for High-throughput Microarray Analysis at the Johns Hopkins University School of Medicine.

CNV Detection:

BeadStudio (I lumina Inc., San Diego, Calif.) software routines were applied to normalize the SNP array data and export signal intensity (R value) and SNP location information for each SNP probe. DNA abundance was calculated as the geometric mean of the signal intensities from each allelic pair, R=(IA2+IB2)1/2, so that the logged R-ratio, Rlr=log2(Rtumor)-log2(Rnormal) represented log fold copy number. Circular Binary Segmentation (CBS), as implemented in the Bioconductor R package, DNAcopy, was applied to estimate the boundaries of segments of constant copy number, and to calculate the mean log fold copy change estimate for each such segment (see, e.g., reference 31). The hybrid approach was adopted to control the amount of smoothing, using sensitive settings in the CBS algorithm in order to detect small, focal events. A second smoothing algorithm was used to combine adjacent segments if the difference in mean log fold copy change was less than 0.25, and the intervening segment of normal copy number covered less than 10% of the total genomic region spanned by the segments under consideration, to prevent excessive segmentation of much larger changes.

Statistical Significance Analysis of Genomic Amplifications and Deletions:

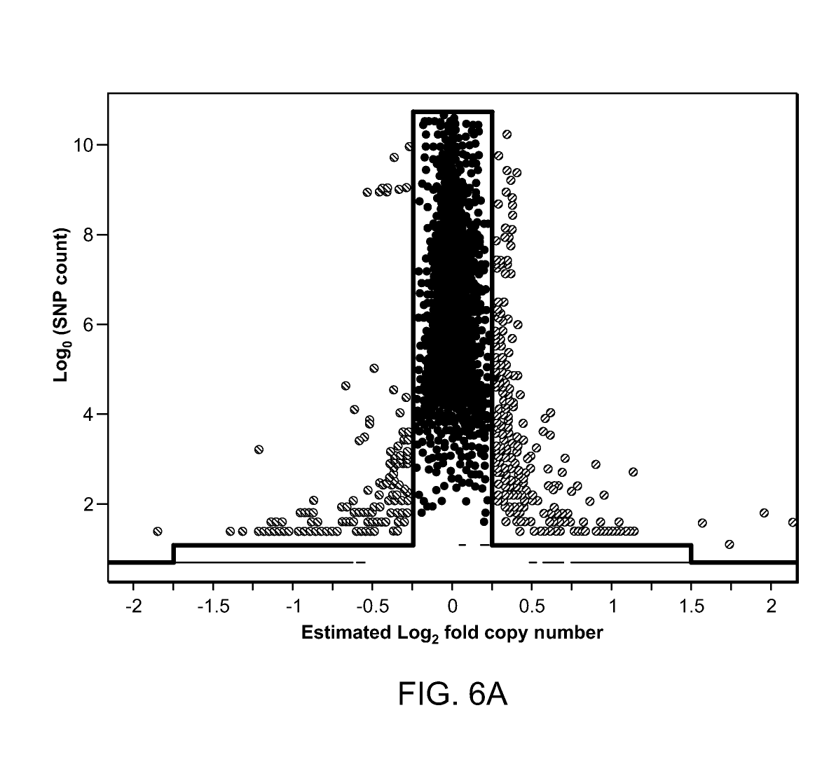

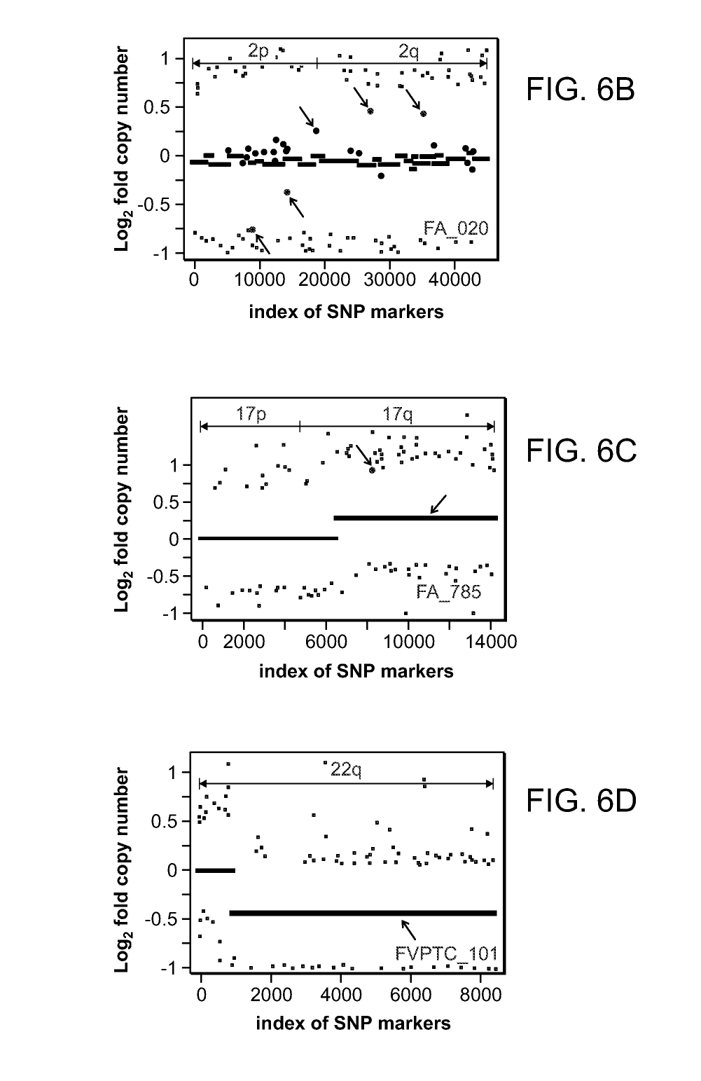

Statistically significant changes were identified by comparing the observed, segmented copy number changes to a null distribution obtained by permuting genomic locations and repeating the segmenting and smoothing steps. Segments of a given log fold copy number change were deemed significant if they extended over a sufficient number of SNPs, selected to control type I error rates at no more than 10%. Specific segment length criteria were derived for log fold changes above 0.25 and below −0.25, as illustrated in FIG. 6. Segments consisting of 3 adjacent SNP tags that had log fold copy numbers beyond ±0.25 were deemed significant, and for log fold changes larger than 1.5, 2 adjacent SNPs were deemed sufficient.

Real-Time Quantitative PCR (qPCR):

Reactions were preformed in triplicate using 1 ng of genomic DNA in a 150 reaction that contained 1 μM of each amplification primer in Real-time SYBR PCR Master Mix (Bio-Rad). Samples were amplified on an Applied Biosystems 7900HT Sequence Detection System and the data was collected and analyzed with SDS 2.3 software. Standard curves were constructed using serial two-fold dilutions of genomic DNA from a normal individual and used to estimate the PCR amplification efficiency, which was confirmed at >97% for each gene to insure the comparability with reference genes. The DNA content of each sample for target genes was normalized to that of Alu, a repetitive genomic element for which the copy number per haploid genome is similar among all human cells (see, e.g., reference 32). Each sample was run in triplicate to ensure quantitative accuracy, and the medians of the threshold cycle numbers (Ct) were taken. The relative copy number changes in the thyroid tumor/normal pairs were reported as T:N ratios and calculated using the 2-AACt method (see, e.g., reference 33). A 130 by Ch21 segment (Ch21: by 27423633-27423762) was chosen for Real-time PCR analysis to compare 3 DNA samples obtained from Down Syndrome patients (Ch21 trisomy) to a DNA sample with normal copies as a genomic amplification control; and a 87 by chromosome X segment (ChX: by 12057855-12057941) to compare normal thyroid tissue samples from 9 males and from 3 females as a genomic hemizygous deletion control.

Real-Time Quantitative PCR of FNA Samples:

All FNA samples were obtained intraoperatively after surgical isolation of the target lesion. All samples were collected with Institutional Review Board approval as part of an ongoing research protocol. The samples were placed immediately into 95% ethanol and stored at −20° C. A total of 18 FNA samples that matched FA tissue samples in this study were available for the subsequent assays. The FNA samples were enriched for epithelial cells using magnetic beads coated with anti-human epithelial antigen antibodies provided in the Dynal Epithelial Enrich kit (Life Technologies, Grand Island, N.Y.) in accordance with the manufacturer's instructions. Genomic DNA was isolated using Lyse and Go PCR reagent according to the manufacturer's instructions (Thermo Scientific, Rockford, Ill.). For the real-time PCR, the same primer sets (see Table 5 below) and amplification protocol as used for thyroid tissue samples were used to assay genomic DNA from the FNA samples. The normalized Ct value (i.e., -delta Ct(Target-Alu)) was calculated to represent the copy number relative to internal Alu sequence signal in thyroid FNA samples. For reference, 3 white blood cell samples from patients with benign thyroid disease (multinodular hyperplasia) were used as normal control of Ch12 copy numbers.

TABLE 5
Primer sequences for genomic qPCR. Chromosomal locations are listed as defined
in the March 2006 human reference sequence (NCBI Build 36.1). The sequences
are listed in 5′ to 3′ orientation.

| | | | | | Annealing |
|---|---|---|---|---|---|
| Gene | Forward | Reverse | Location | Size | temp. |
| GPD3 | ACACCTGTGCCAG | TGACGGTGGCAGA | chr12:7734036-7734177 | 142 bp | 63° C. |
| | ACTAAGATGCT | GGTTCTTACAA | | | |
| GPD3 | GGGACTGACCGCA | AAAGGGAACAGTT | chr12:7734318-7734483 | 166 bp | 68° C. |
| | ACACAAACATT | GACATTGGCCC | | | |
| GPD3 | TGGCCAACAACAC | TGTGGTGAGCCGA | chr12:7736231-7736345 | 115 bp | 66° C. |
| | CTGACTGTCTA | TATCACACCAT | | | |
| FGD6 | TGCACAAGCGAAT | AGCCTGGAGACAG | chr12:94010555-94010662 | 108 bp | 63° C. |
| | TCACTCTCACC | TAAAGACCACA | | | |
| FGD6 | TTGGTAGAGTTGC | AAGGCCTGTGAGG | chr12:94010015-94010100 | 86 bp | 64° C. |
| | AGAGACGTGGT | TATACTGATCACC | | | |
| FGD6 | AGCAGGACTGCTC | TACGAGAATCGCT | chr12:94008914-94009091 | 178 bp | 62° C. |
| | AGGTCTATGTT | TGAACCCGAGA | | | |
| NDUFA12 | AGGCAAGATGGAG | CCTTCCAAGAAAT | chr12:93921436-93921594 | 159 bp | 64° C. |
| | TTAGTGCAGGT | CAGCCAGCGAA | | | |
| NDUFA12 | ACTGCCGTACAGT | AACTATGCTGCTC | chr12:93921092-93921185 | 94 bp | 63° C. |
| | TCCTTGTCTGT | GTGGGATCAGT | | | |
| NDUFA12 | AGTAAACAGCCAA | GGCCGACAGAGAC | chr12:93920324-93920489 | 166 bp | 62° C. |
| | TGAAGGTATGGA | TCCATCTCAAA | | | |
| NR2C1 | AGGCCCAGTGTCT | CTTTGCAGCAGGC | chr12:93953752-93953856 | 105 bp | 66° C. |
| | GTAAATTGGGA | AATGGCTTAGA | | | |
| NR2C1 | TCTCATCTGCCAC | GCTGGCTTGTGCT | chr12:93953386-93953524 | 139 bp | 62° C. |
| | TGGTGTCTT | ATGCATCTTGT | | | |
| NR2C1 | TCCTCACCTCTTC | GGCCACAAGAAAC | chr12:93952174-93952357 | 184 bp | 62° C. |
| | CTCAATTCTG | TGCCTGTCATT | | | |
| VEZT | TTGCCCACTCACA | AAATGATGGTGGC | chr12:94194829-94194978 | 150 bp | 67° C. |
| | TCCAGTCTGTT | TGGGACTAGCA | | | |
| VEZT | CCTGACTGACTAG | GGGTACCCATTAT | chr12:94195571-94195723 | 153 bp | 63° C. |
| | CCATTTGCCTT | ATGTCAAGCCC | | | |
| VEZT | TGACTACTGTGTG | AGTCTCACATTTC | chr12:94195973-94196156 | 184 bp | 64° C. |
| | GTCCTGAGCAA | AGAGCAGGCCA | | | |
| Alu | AGAGTCTCACTCT | GAGGCACGAGAAT | AluSx_5 region | 92 bp | 60° C. |
| | GTAGCCCAA | CGCTTGAG | | | |
| NA | GTCCATGCAGGAA | CATGAGGCTTGAA | chr21:27423633-27423764 | 132 bp | 59° C. |
| | AAGGAAG | CCATGTG | | | |
| NA | ATTCCTGCCCCAT | GCCCCACATTGGT | chrX:12057855-12057941 | 87 bp | 60° C. |
| | AGGATTG | ATAATGC | | | |

RNA Isolation and Expression Array Analysis:

RNA samples were prepared from the same 39 thyroid tumor-normal tissue samples used for SNP arrays, using the Qiagen RNeasy Kit (Qiagen, Valencia, Calif.). The quantity and integrity of extracted RNA was evaluated by ND-1000 Spectrophotometer (Nanodrop Technologies, Wilmington, Del.) and Bio-Rad Experion RNA Assay (Bio-Rad, Hercules, Calif.), respectively. Microarray hybridizations were performed in the Microarray Core Facility at Johns Hopkins University School of Medicine. For each sample, 500 ng total RNA was used for transcriptome analysis using the HumanHT-12 v3 Expression BeadChip kit (Illumina, San Diego, Calif.), which targets ˜25,000 annotated genes with more than 48,000 probes. Arrays were processed as per the manufacturer's instructions. Hybridization signals were analyzed using BeadStudio Gene Expression Module v.3 (Illumina) (see, e.g., reference 34). Quantile normalization and statistical analysis of the gene array data were carried out using the Limma (see, e.g., reference 35) package and customized scripts in R/Bioconductor (see, e.g., reference 36).

### REFERENCES

1. Lubitz C C, Faquin W C, Yang J, Mekel M, Gaz R D, Parangi S, Randolph G W, Hodin R A, Stephen A E: Clinical and cytological features predictive of malignancy in thyroid follicular neoplasms, Thyroid 2010, 20:25-31.
2. Zeiger M A: Distinguishing molecular markers in thyroid tumors: a tribute to Dr. Orlo Clark, World journal of surgery 2009, 33:375-377.
3. Nikiforov Y E: Molecular diagnostics of thyroid tumors, Archives of pathology & laboratory medicine 2011, 135:569-577.
4. Nikiforov Y E, Steward D L, Robinson-Smith T M, Haugen B R, Klopper J P, Zhu Z, Fagin J A, Falciglia M, Weber K, Nikiforova M N: Molecular testing for mutations in improving the fine-needle aspiration diagnosis of thyroid nodules, J Clin Endocrinol Metab 2009, 94:2092-2098.
5. Ohori N P, Nikiforova M N, Schoedel K E, LeBeau S O, Hodak S P, Seethala R R, Carty S E, Ogilvie J B, Yip L, Nikiforov Y E: Contribution of molecular testing to thyroid fine-needle aspiration cytology of “follicular lesion of undetermined significance/atypia of undetermined significance”, Cancer Cytopathol 2010, 118:17-23.
6. Yip L, Kebebew E, Milas M, Carty S E, Fahey T J, 3rd, Parangi S, Zeiger M A, Nikiforov Y E: Summary statement: utility of molecular marker testing in thyroid cancer, Surgery 2010, 148:1313-1315.
7. Brunaud L, Zarnegar R, Wada N, Magrane G, Wong M, Duh Q Y, Davis O, Clark O H: Chromosomal aberrations by comparative genomic hybridization in thyroid tumors in patients with familial nonmedullary thyroid cancer, Thyroid: official journal of the American Thyroid Association 2003, 13:621-629.
8. Castro P, Eknaes M, Teixeira M R, Danielsen H E, Soares P, Lothe R A, Sobrinho-Simoes M: Adenomas and follicular carcinomas of the thyroid display two major patterns of chromosomal changes, The Journal of pathology 2005, 206:305-311.
9. Dettori T, Frau D V, Lai M L, Mariotti S, Uccheddu A, Daniele G M, Tallini G, Faa G, Vanni R: Aneuploidy in oncocytic lesions of the thyroid gland: diffuse accumulation of mitochondria within the cell is associated with trisomy 7 and progressive numerical chromosomal alterations, Genes, chromosomes & cancer 2003, 38:22-31.
10. Finn S, Smyth P, O'Regan E, Cahill S, Toner M, Timon C, Flavin R, O'Leary J, Sheils O: Low-level genomic instability is a feature of papillary thyroid carcinoma: an array comparative genomic hybridization study of laser capture microdissected papillary thyroid carcinoma tumors and clonal cell lines, Arch Pathol Lab Med 2007, 131:65-73.
11. Frisk T, Kytola S, Wallin G, Zedenius J, Larsson C: Low frequency of numerical chromosomal aberrations in follicular thyroid tumors detected by comparative genomic hybridization, Genes, chromosomes & cancer 1999, 25:349-353.
12. Hemmer S, Wasenius V M, Knuutila S, Joensuu H, Franssila K: Comparison of benign and malignant follicular thyroid tumours by comparative genomic hybridization, Br J Cancer 1998, 78:1012-1017.
13. Miura D, Wada N, Chin K, Magrane G G, Wong M, Duh Q Y, Clark O H: Anaplastic thyroid cancer: cytogenetic patterns by comparative genomic hybridization, Thyroid: official journal of the American Thyroid Association 2003, 13:283-290.
14. Roque L, Nunes V M, Ribeiro C, Martins C, Soares J: Karyotypic characterization of papillary thyroid carcinomas, Cancer 2001, 92:2529-2538.
15. Roque L, Rodrigues R 392, Pinto A, Moura-Nunes V, Soares J: Chromosome imbalances in thyroid follicular neoplasms: a comparison between follicular adenomas and carcinomas, Genes, chromosomes & cancer 2003, 36:292-302.
16. Singh B, Lim D, Cigudosa J C, Ghossein R, Shaha A R, Poluri A, Wreesmann V B, Tuttle M, Shah J P, Rao P H: Screening for genetic aberrations in papillary thyroid cancer by using comparative genomic hybridization, Surgery 2000, 128:888-893; discussion 893-884.
17. Wreesmann V B, Ghossein R A, Hezel M, Banerjee D, Shaha A R, Tuttle R M, Shah J P, Rao P H, Singh B: Follicular variant of papillary thyroid carcinoma: genome-wide appraisal of a controversial entity, Genes, chromosomes & cancer 2004, 40:355-364.
18. Wreesmann V B, Sieczka E M, Socci N D, Hezel M, Belbin T J, Childs G, Patel S G, Patel K N, Tallini G, Prystowsky M, Shaha A R, Kraus D, Shah J P, Rao P H, Ghossein R, Singh B: Genome-wide profiling of papillary thyroid cancer identifies MUC1 as an independent prognostic marker, Cancer research 2004, 64:3780-3789.
19. Lloyd R V, Erickson L A, Casey M B, Lam K Y, Lohse C M, Asa S L, Chan J K, DeLellis R A, Harach H R, Kakudo K, LiVolsi V A, Rosai J, Sebo T J, Sobrinho-Simoes M, Wenig B M, Lae M E: Observer variation in the diagnosis of follicular variant of papillary thyroid carcinoma, Am J Surg Pathol 2004, 28:1336-1340.
20. Elsheikh T M, Asa S L, Chan J K, DeLellis R A, Heffess C S, LiVolsi V A, Wenig B M: Interobserver and intraobserver variation among experts in the diagnosis of thyroid follicular lesions with borderline nuclear features of papillary carcinoma, American journal of clinical pathology 2008, 130:736-744.
21. Ghossein R: Encapsulated malignant follicular cell-derived thyroid tumors, Endocrine pathology 2010, 21:212-218.
22. Peiffer D A, Le J M, Steemers F J, Chang W, Jenniges T, Garcia F, Haden K, Li J, Shaw C A, Belmont J, Cheung S W, Shen R M, Barker D L, Gunderson K L: High-resolution genomic profiling of chromosomal aberrations using Infinium whole-genome genotyping, Genome Res 2006.
23. Olshen A B, Venkatraman E S, Lucito R, Wigler M: Circular binary segmentation for the analysis of array-based DNA copy number data, Biostatistics 2004, 5:557-572.
24. Hartigan J A: Clustering algorithms. Edited by New York, N.Y., USA, John Wiley & Sons, Inc., 1975.
25. Hanley J A, McNeil B J: The meaning and use of the area under a receiver operating characteristic (ROC) curve, Radiology 1982, 143:29-36.
26. Sobrinho-Simoes M, Eloy C, Magalhaes J, Lobo C, Amaro T: Follicular thyroid carcinoma, Modern pathology: an official journal of the United States and Canadian Academy of Pathology, Inc 2011, 24 Suppl 2:S10-18.
27. Mazzucchelli L, Burckhardt E, Hirsiger H, Kappeler A, Laissue J A: Interphase cytogenetics in oncocytic adenomas and carcinomas of the thyroid gland, Human pathology 2000, 31:854-859.
28. Hemmer S, Wasenius V M, Knuutila S, Franssila K, Joensuu H: DNA copy number changes in thyroid carcinoma, The American journal of pathology 1999, 154:1539-1547.
29(S1). De Lellis R A, Lloyd R V, Heitz P U, Eng C E: Pathology and Genetics: Tumors of Endocrine Organs. Edited by Lyon, France, IARC Press, 2004, 30(S2). Liu Y, Sun W, Zhang K, Zheng H, Ma Y, Lin D, Zhang X, Feng L, Lei W, Zhang Z, Guo S, Han N, Tong W, Feng X, Gao Y, Cheng S: Identification of genes differentially expressed in human primary lung squamous cell carcinoma, Lung Cancer 2007, 56:307-317
31(S3). Olshen A B, Venkatraman E S, Lucito R, Wigler M: Circular binary segmentation for the analysis of array-based DNA copy number data, Biostatistics 2004, 5:557-572
32(S4). Walker J A, Kilroy G E, Xing J, Shewale J, Sinha S K, Batzer M A: Human DNA quantitation using Alu element-based polymerase chain reaction, Analytical biochemistry 2003, 315:122-128
33(S5). Livak K J, Schmittgen T D: Analysis of relative gene expression data using real-time quantitative PCR and the 2(-Delta Delta C(T)) Method, Methods 2001, 25:402-408
34(S6). Goring H H, Curran J E, Johnson M P, Dyer T D, Charlesworth J, Cole S A, Jowett J B, Abraham L J, Rainwater D L, Comuzzie A G, Mahaney M C, Almasy L, MacCluer J W, Kissebah A H, Collier G R, Moses E K, Blangero J: Discovery of expression QTLs using large-scale transcriptional profiling in human lymphocytes, Nature genetics 2007, 39:1208-1216
35(S7). Smyth G K: Linear models and empirical bayes methods for assessing differential expression in microarray experiments, Statistical applications in genetics and molecular biology 2004, 3:Article3
36(S8). Gentleman R C, Carey V J, Bates D M, Bolstad B, Dettling M, Dudoit S, Ellis B, Gautier L, Ge Y, Gentry J, Hornik K, Hothorn T, Huber W, lacus S, Irizarry R, Leisch F, Li C, Maechler M, Rossini A J, Sawitzki G, Smith C, Smyth G, Tierney L, Yang J Y, Zhang J: Bioconductor: open software development for computational biology and bioinformatics, Genome Biol 2004, 5:R80

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method for molecularly characterizing a thyroid lesion, the method comprising detecting in a biological sample of the lesion characteristic DNA copy number variation at one or more of chromosomes 7, 12 and 22, thereby characterizing the lesion as having benign or malignant potential.

2. The method of claim 1, wherein the method identifies a characteristic DNA copy number variation that could not be identified by karyotyping.

3. A method for characterizing a thyroid lesion, the method comprising detecting in a biological sample of the lesion characteristic DNA copy number variation at one or more of chromosomes 7, 12 and 22, wherein said detection is by one or more of SNP array analysis, PCR analysis, hybridization, fluorescence in situ hybridization, quantitative Real-time genomic PCR analysis, gene expression array analysis, or transcriptome array analysis, thereby characterizing the lesion as having benign or malignant potential.

4. A method for molecularly characterizing a thyroid lesion, the method comprising detecting in a biological sample of the lesion characteristic DNA copy number variation at one or more of chromosomes 7, 12 and 22, thereby characterizing the lesion as a benign follicular adenoma, a classic papillary thyroid carcinoma or a follicular variant papillary thyroid carcinoma.

5. The method of any one of claim 1-4, wherein the method further comprises detecting a mutation in a Ras gene.

6. The method of claim 5, wherein the mutation is H-ras or N-ras.

7. The method of any one of claims 1-4, wherein the method further comprises detecting an increase in telomerase expression or activity.

8. The method of claim 7, wherein telomerase expression is detected in an HTERT assay.

9. The method of claim 1, wherein the molecular characterization is not by karyotyping.

10. The method of any of claims 1-4, wherein said detection is by one or more of SNP array analysis, PCR analysis, hybridization, fluorescence in situ hybridization, quantitative Real-time genomic PCR analysis, gene expression array analysis, or transcriptome array analysis.

11. The method of claim 3, wherein the characteristic DNA copy number variation is a segmental amplification at chromosome 12 that is indicative of a follicular adenoma.

12. The method of claim 11, wherein the method distinguishes a follicular adenoma from a classic papillary thyroid carcinoma or a follicular variant papillary thyroid carcinoma.

13. The method of claim 11, wherein the characteristic DNA copy number variation is chromosome 12 amplification that identifies the lesion as being benign or as having no or little malignant potential.

14. The method of claims 1-4, wherein amplification at chromosome 12 is detected by measuring the expression or activity of any one or more markers selected from the group consisting of NDUFA12, NR2C1, FGD6, VEZT, M1R331, RPL29P26, LOC729457, METAP2, USP44, CD163L1, LOC727815, BICD1, FGD4, DNM1L, YARS2, UTP20, ARL1, SPIC, WNK1, DRAM, RAD52, HSPD1P12, CERS5, LIMA1, MYBPC1, CHPT1, SYCP3, PKP2, CCDC53, HAUS6, PLIN2, LOC729925, YPEL2, DHX40, CLTC, PTRH2, TMEM49, MIR21, TUBD1, PLIN2, RPS6 KB1, HEATR6, LOC645638, LOC653653, LOC650609, CA4, USP32, SCARNA20, C17orf64, and APPBP2.

15. The method of claims 1-4, wherein amplification at chromosome 12 is detected by measuring the expression or activity of any one or more markers selected from the group consisting of NDUFA12, NR2C1, FGD6, VEZT, MIR331, RPL29P26, LOC729457, METAP2, USP44, and CD163L1.

16. The method of claims 1-4, wherein amplification at chromosome 12 is detected by measuring the expression or activity of any one or more markers selected from the group consisting of NDUFA12, NR2C1, FGD6, VEZT and GDF3.

17. The method of any of claims 1-4, wherein the characteristic DNA copy number variation is a chromosome 22 deletion, and presence of the deletion is indicative of a premalignant state leading to invasive disease.

18. The method of any of claims 1-4, wherein the biological sample is a tissue sample, biopsy sample, or fine needle aspirant.

19. The method of any of claims 1-4, wherein RNA or genomic DNA is isolated from the sample prior to analysis.

20. A method for distinguishing a follicular adenoma from other thyroid lesions, the method comprising detecting in a thyroid lesion a segmental amplification in chromosomes 7 and 12, wherein the presence of said amplification at chromosomes 7 and/or 12 is indicative that the lesion is a follicular adenoma.

21. The method of claim 21, wherein detection of the amplification on chromosome 12 indicates that said follicular adenoma is unlikely to progress to thyroid cancer.

22. A method for distinguishing adenomatoid nodules or follicular variant papillary thyroid carcinoma from other thyroid lesions, the method comprising detecting in a thyroid lesion a chromosome 12 amplification, wherein the presence of the chromosome 12 amplification is indicative of adenomatoid nodules or follicular variant papillary thyroid carcinoma.
